SEQUENCE LISTING (6 sequences)

SEQ ID NO: 1 - protein, 142 aa, Homo sapiens
VSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 2 - protein, 142 aa, Mus musculus
VSEPTTVPFDVRPGGVVHSFSQDVGPGNKFTCTFTYASQGGTNEQWQMSLGTSEDSQHFTCTIWRPQGKSYLYFTQFKAELRGAEIEYAMAYSKAAFERESDVPLKSEEFEVTKTAVSHRPGAFKAELSKLVIVAKAARSEL

SEQ ID NO: 3 - protein, 173 aa, Homo sapiens
MAAPSGGWNGVGASLWAALLLGAVALRPAEAVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 4 - protein, 166 aa, Mus musculus
MAAPSGGFWTAVVLAAAALKLAAAVSEPTTVPFDVRPGGVVHSFSQDVGPGNKFTCTFTYASQGGTNEQWQMSLGTSEDSQHFTCTIWRPQGKSYLYFTQFKAELRGAEIEYAMAYSKAAFERESDVPLKSEEFEVTKTAVSHRPGAFKAELSKLVIVAKAARSEL

SEQ ID NO: 5 - DNA, 12847 bp, Homo sapiens
acgtgtccctcgccgcgccccgtctacccgcccctgccctgaggaccctagtccaacatggcggcgcccagcggagggtggaacggcgtcggcgcgagcttgtgggccgcgctgctcctaggggccgtggcgctgaggccggcggaggcggtgtccgagcccacgacggtggcgtttgacgtgcggcccggcggcgtcgtgcattccttctcccataacgtgggcccgggggtacgtgccaccgccgtcccggatatttgagtgctggcaggcccggggagggggcgcggagcattgggggcggggcccgaggagggggcgcggaccgcggaggtgggtactgaagaaggggtgcggattgttagggtcggaggctgagtggggggcgcgggaccgtgaggccggggtcatcactggggcaggactgaactcatagggagcggggccttgtgggccccggcggcgttgagggggcgtgatcacctgtcacctgtcgcctgcctggagtgtccaggcacccctgccctccagacgcagaggtggggaggggtcagtgatgggagcggcaggaggctaaagggcatctgccctcctgggcctgaaggaaatcagtcgctgcagctccgaagagggaggaagtgctccttccttccattgcacaaggtcccccctcaccgagggtgtgaggccgggccctcagccctgtactcagcttgtcggcaaagtcctttcccctctcagcctccctttccccgcctgtaataacagtatctttactgttttttgactatttctttttagacttgcgtgcagtggcgggatctcggctcactgcatccttagccttctgggttcaagcaattcttctgcctcagcctccccagtacctgggattacagacgtgtgccaccacacccggataattgttgttgttgttgtttgagatggagcctcactctgtcgcccaggctggagtgcagtggcgcgatcttggctcactgcaacctctgcctcccaggttcatgccattctcctgccgcagcctcccaagtagctgggactactaatctttgtatttttagtacagatgggatttcaccatgttagccaggctggtcttgaactcctgaccgcaggtgacccgcccacctcggcctcccaaagtgatgggattacaggcgtgagccactgtgccaggccagtttgttgactacttccttatagttaggtgtgatgccaagtcctttatatgttaggcccaagttacagatgagaaaatgcttcacataaaaaagagaaacagagagaaactagaggctcagaaaagttttcacttgtccaggtcccatggcaaaacccatctctacaaaaatacgaaaaattagccaggtgtggtagcaggtgcctgtaatcccaagtactcaggaggctgaggcaggagaatcgtttgaacccgggaggcagaagttgcagtgagctgagatcatcccacttcagcctgggcgacagaacaagactctgcttcaaaaaaaaaaaaaaaaattagtcgggtgtggtggttgcgtacctattgtctcagttacttgagaggctgaggcaagaggattactcaaacctgggagttcgaggctgcagtgagctgtcattgtgcccctgcactccagcttgggtgacagcaagaccccgtctcttaaacaaaataaaaatttccttctaccaaaccttttttttccttccattctctaggacaaatatacgtgtatgttcacttacgcctctcaaggagggaccaatgaggtgagttgttcaaaattccctctagcttactgtgacagtatccattgttgtagggagatgttctgtagcagacagcagggttgggtctcacctatatcaccttacactcattgaatgactcttcctaaaccaataaaaagggagagtattggcattaaacagtgacgacagattctgctgttattattctacagagcaagcaaatgtctggacagaaggagggaggggagatgggagtctgccatccccatgagcatctgttgggatgttgtattttatgtgcagtgcaacaagttatcacagcttaggggcttaaaacagcacccatttattatctcatagctttgtaggtcagaagtctgggtcagctcagttgtgtgcttcataaaaccaaactcaaggtattttgggaggctgaggcgggcagatcacctgaggtcaaagttcgagaccagcctggccaacatggcaaaacctcatctctactaaaaatacaaaaattagctgggcatggtggcacatgcctgtagtcccagctactcaggaggctgaggcaggagaatcacttgaacccaggagacggaggttgcagtgagccaagattgtgccactgcactccagcctgggcaacagagcgagactctgtctcaaaaaataaataataaataaataaaggtatcacaggcaaggcatggtggctcatgccccatctctacagaaaataaaaaaattagcccagcgtggtggcgtgcacctgtggttccagctacttgggagactgaggtgggaggatcgcttgagcccaggaggtcaaggctgtagtgagccgtgattgtgctactgggcaacagagcaagaccctgtctcaaaaaaaaaaaaagtctctgggctgggagaagaatcctctccttcaagctcttagttggcagaattcagttacttgtggttcagggaccaaggtcccggtttccttgctggctgttgtctccagctggtggcagaagagccagtaaagatgatttgggccaggcacagtggctcatgcctataatcccagcactttgggaggccaaggctggcgaatcacctgaagtcaggagctccagaccagcctggccaacatggtgaaaccccgtctctactaaaactacaaaaattagccgggcgagccaggcgcactggctcatgcctgtaatcccagcactttgggaggccgaggcgggtggatcacaaggtcaggagatcgagaccatcctggctaacacggtgaaaccccgtctctactaaaaatacaaaaaattagccgggtgttttggtgggcacctgtagtcccagctactggggaggctgaggcaggagaatggcgtgaacccgggaggcggagcttgcagcgagccaagatcgcgccactgcactccatcctgggcgacagagcgagactctgtctcaaaaaaaaaaaaaaaaaaaaaaaaagggtgatttgaagcacatggacacatacgccactgctgcccttgagggaactgcagggcagggaatacacgcggcctctaggagctgagaacagcctctggctggcagcaaggaaatgaagacttcagtcccgcaaccaagaggaactgaaatctgccacaaccacaagagcttggaagaggaccttgagctaccagtaagagcctagcccggtcaacaccttgagatgggtttcgccatgttggccaggctgatcttgaactgctgacctcaaatgatcagcctgcctcggcctcccaaagtgctgggattacaggtgtgagccaccggtgcccggcctcctagagatttttctgtatcctatgtaaagcgcttcagttgcatggacgtgtggagttgacaatcccgtactaatggattgagggttacttccaacccagtccgtgaataaccttggccagggttcattgtacaagtgcagttacatctgtgggaaaaccacccagaagtgggattcatgggtccattttcttaaaagtaaaactgggctgggcgcggtggctcacgcctgtaatcccagcactttgagaggccaaggcaggtggatcgcctgaggttgggagtttgagaccagccttgctaagatggtgaaaccccatctctactgaaaatacaaaagttagcagggcgtggtggcacgcgcctgtaatcccagctactcaggaggctgaggcaggagaatcgcttgaacccacgaggcagaggttgcagtgagctgagatcgcaccactgcactccagcctgggtgacagagcgagactccatctcaaaaaaaaaaaaaaaagtaaaactgtatgttttgagatcatggtagatttacattcacttgtaagaaatcctacagagagatccaagtaccgtttaaccaggttccccggtggtaacatcgtgcaagaggccagtcattttagagatggggtctcgctatgttgcccaggctggtctcgaactcttgggttcaagtgatcatcccacgttggcctcccaaagtgctgggattacaggcatgagccaccatgcccagcctgcattttccattttgcttgtacctgttgcagctccaccgacaacctggaatagtcttggttttcacacccatatggtgtgttagcagacaggagggcttttgcccttctgacctgggagaaaaaggtatctcacgatagttcagcttgtgtttatttatttttcataaatgatatgggacatctttttttttttttttttttttttttttttttgagacagagtctcgctctgtcgcccaggctggagtgcagtggcgcgatctcagctcactgcaagctccgcctcccgggtacacgccattctcctgccttagcctcccgagtagctgggactacaggcgcccaccactgcgctcggctaattttttgtatttttagtagagatggggtttcatcatgttagccaggatggtcttgatctcctgacctcgtgatctgcctgcctcggcctcccaaagtgctgggattacaggcgtgagccaccgcgcccggcctggacatctatttatatgtttaagagttgttcttaggcctggcatggtggcttatatctgtaatctcagcactttgggaggccaaggtgggaggattgcttgaggccaggaggttgagaccagcctgggcaatatggcaagacagtgattctacaaaacacaaaaattagctgggtgtggtcccagctacttgggaggctgaggtgggaggatcgcttgagcccaggaggtagaggttgcagtgaggcgagatcgtgccactgcacgccagcccaggccacagagagagactccatctcaaaaacaaacaaacaaacaaacaaacagcgttgttcctatttccccatccgtgggagccactttggggtagttctcattagaggcaccacttttatgggaaggatgtggtgcaggcaggctccctgatcacagatgacagcaaaccccaggccaccaggagggcggggcaggggatttagtggccagcccctgaatcgggaggtacagaagaggtggcctgactgtagaatcagaggcacacctagaagtctccgagagcagctgtgtgtccggggctgacccgcgctgtcttctctccccagcaatggcagatgagtctggggaccagcgaagaccaccagcacttcacctgcaccatctggaggtgagactcggggtggggatggcatttccggctgccgttggcctgccctgtggcctcaggagctgctttggaaggcagggaacgagggctgcacccctggaggggaagacagaccagcagggagactcatgcacgtgcaggtggggggtggcagggtcgctggggtgggagtgctccccaaggaagtgcactgatgcagagactgagaaggtctgggggagacggggggagcctgtgcggggcaaggtcccaggaaaaggagaagcagctcagagggtgcggagcgtggaccagggcccaggaaggccccaaggggctccaccctcccctaagggtgagacgaactcaccaaagggctttttggcaatttttttttttttttttttgagatggagtctccatcttttgcccgggctgtagtgcagtggagcaatcttggctcactgcagcctccgcctcctgggttcaagcgattctcctccctcagcctcctgagtagctgggattacaggcgtgtgccaccatgcctgaccctttttggcaatgtttaaaaactgagataaaattccataaagcacagcattttaagctcacaattcagctgcatggagtactttctcagaggtgtgcagccagacctctgcttccagaacattttcatcccctgaaaataaactctgttcccatcagagtcattctccttcccagcacatggcagccacgcatccccctcctgtctctgtggatgggcgtgtcctggacattgcatagaaatggggtcaaaccttccctgtgtggcctttggtgtctggcgtctctcactgaacgtgacgtcctcaaggctcatccatgctgaggcccgtgtcagcctcatttcttttcctggctgagtcctattccagagggctccctgcaggaacatgtggagctcagatccccccggctgaaggaggcaggctgaggcagggagcccctccaggcaccaaccaggatggagtcgggggttgggggtggggcgcagattgaagcctgtgcagaatgaggatggggtgggtggagagggcacagattggaggctgtgtagaatgaggatggggtgggtggggagggtagattggaggctttgtagaatgaggatggggtgggtggggagagcacagattggagactgtgtagaatgaggacagggtgggtggggagggtatactggaggctgtagaatgaggatgaggtggggagggtagattggaggctgtagaatgaggatggggtgggtggggagggtagattggaggctgtgtagaatgagggtggggtgggtggggaggatagattggaggctgtgtagaatgaggatggggtgggtggagagtgcacaggttggaggctgtagaatgaggatggggtggggagggtagattggaggctgtgtagaatgaggatggggtgggtggagagggcacagattggaggctgtgtagaatgaggatggggtaggtggggagagtagattggatgctgtagaatgaggatggggtggatgggagcgtagattggaggctgtgtagaatgaggatgggatgggtggagagggcacagattggaggctgtagaatgaggatggggtgggtggggagggtagattggaggctgtgtagaatgaggacaggacgggtggggagggtagattggaggctgtgtagaatgaggacaggacgggtggggagggtagattggaggctgtagaatgaggatgaggtgggtggggaatgtagatcggaggctgtagaatgaggatggagtggggtggggagagtagattggaggctgtagaatgaggacggagtgggaggggaaggcacagactggaggctgtgtaaaatgaggacagggaggaggggagggcacagattggaggctgtggaatcagctagctggggtaggaggaggaggaaggtgctgggcagagtcctggtggaactggcggagctggtgcaggtctgggctggggacgagctaggcaggtggcaggtgtgggagtctgtggggtagtggctgtggaggtttggatgatgctatctcccggggaatgtgtcccaccagaaggggccccagctgtttcccctcaggcaggtctttaccagcatgttctctgaaggaccagcctgatggctcatagccttcttggggtgcagacatctgggaatcagacaaaagctgtgcactctcttccacctgcccgaacacacatgcatctgggtccacagcccctccccaagtctccccagacaccgcccgattctgcccctgccgccatcctctgtcaggctcatggtttatgggcttatcccggctccactcgccccactcattctcgtaaccccactgcctagatcccagctcgatctgggcaaggtcatgaagggaaggaagccgtcaggccccccggtggggagggggtcgggggcttcaggaatcttgtcccttcttccctgatccttggcccagaatgtctcagcccaaacagtgggtatttcttgagcctttattttgtgcagacactttggagggagaagtccaagctatagctccaccactgcggtcaggttaaagcacgttgacaaatgggaggcagagatcagggagtggcgacgtttaactgaaggacagggtgtgatttgctgcctcgtgtgggtgttgtcccgccgtgccctgcggggcgctttacaaacagcaggtgtttctggggagattctcacccactccatggaactggctgcatcacaggcaggccaaggccccaggaggcatcaggagggtcattgagaggagataggccccatcagcccagagagaccctgcagaggccgccctgtagtcttggggaagcccacggtcgccatcgtctgtctgcaggccactgccctgcatggacccctgagtgccagctccccagagggcatctgggcaggtgcaggtctggagcagttgggatggtggctgggctcaccttgagcctgaatcctcctgtccaacccccacaaaagctccccaggggcatgctcagccccggagacctgccaccccaggcctcctcatctcagacagtgactggaccctccctgaccagggctgttggcagtgctagggcttaaagaagaatctagggctccctggatacccccgccccacccatgcacatagaaccccagccagggcttccacagaaatggcccccctgctgcccctccatccccatcacactctcttcacaccagagatgccatcatcccctgctgtcaccttccgacaccttctgccactccagttccttagaagcctccgttagcagccttctagagacctgggcagatgatcctccccacttcttaagttaactcctgtacttcctagtcaatgccctctgagaagcctttactggcctctcattgcccagccccagcccccgtcgtagtacctggattgctgtcagcaaccaagaagccatgtgcagtgaatggacatggggtcctgtcccagagcagctcccagcccacaggggagagaggcgtgtctatgaatgagctggaagaacattctggcacaaaggggccaggcacagtggctcacacctctaatcacagccctctgggaggctgaggcgagtggatcacttgaggtcaggggttcaggccaggctggccaacatggtgaaaccatgtttttactaaaaataccaaaattagtcagatgtggtcgcgggtgcctgtactcccagctacaagggaggctaaggcaggagaatcacttaaacccaggaggcagaggctgcagtgagtgagattgtgccattgccatccagcctgggcaacagagtgagactcgtctccaaaaaaaaaaaaaaaaaaaaattctggcacaaaagaaagagggtctgctgttgagagggccccatccatccttcccagtcagcaggtgctcgtgctcgggaatggcgtccatgttgagaagcaggcacgagacctgctgaccccagcccgagtccctggcggggctccttccagggcacacagacccagacccagcaggcctggcctgactccctcgccccctcttcctctgcaggccccaggggaagtcctatctgtacttcacacagttcaaggcagaggtgcggggcgctgagattgagtacgccatggcctacgtgagtatcttggagtggggcaggctccgggtgtggcttctgtgcactggggcagctgtgagatgtcctgccacaagagggctccatggggacagggaggagaggggacagcgaatcctgcagaggtagggtgtgtaacttgcaggaacctggcacacagacgcccccaggccaggggcacgggggtttggtaacctgttgctgctggcttctcttgtatatgccagaaaagacttctccaggccaggcgcggcggttcacgccagtaatcccagcaatttgggaggctgaggcaggaggattgctggaggccaggaatttgattccagcctaggcaacatagcaagaccctatatctaccaaaagtaaaaaaaaaatagccaggcgtgttggcatgcacccatagtcccacctactcaggaggccgcagcaggaggatcacttgagtccaggactttgaagctgcagtgagctatgattgcactgctgcattccagcctggacagtagagtgaggtcctggctcaaaaacaaaaaagtctcctctccatctctagtaaaagtagaaagctttagtctgtgcttctctggaaagcaaactcaatcgttttcctaatgatgagcattgaactggccctggtaaagtttccactcattctttttcagtctaaagccgcatttgaaagggaaagtgatgtccctctgaaaactgaggaatttgaagtgaccaaaacagcaggtacggaaagatggggatggggtagaggtgacgccacccccatccaagggtgggcaatcggggcagtttggagaggggggtcagccataggggtcctggagactgtagaccaggcatccacaaactgattctataaaggccacctcgtcaatatcttcagctttgtggcactgtgggttctgtctagagctgtgccattgtggcacaaagctgctgcagacagtatgtccatgcgtgggtgtggctgtgtgccactagaactttatcttagctgggcgcggtggctcatgcctgttaccgcagctgctcaggaggctaaggtgggaggatcattggagcccaggaggtcaagaccagcctgggcaacatagcgagaccctgtctctacaaaatttttttaaaaatcaggctgggtgcggtggctcacgcctgtaatcccagcactttgggaggacgaggcgggcggaacacttgaggtcaggagttccagaccagctggccaacataatgaaaccccatctctactaaaaatgcaaaaaattagccaggcgtggtggtgcgcacctgtaatcccagctattcaggagaattgctggaacctgggaggtggaggttgcagtgagctgagatcatgccactgcactctagcctggaagacagcaagattctgtctcaaaaagaaaaaaaattagccgggcatggtggtgtgtacctgtagtctcagctacttaggaggctgaggtgggaggattatttgagcccaggaggtcgaggctgcattgagctatgatcgcaccactgcactccagcctgggcatcagagtaagactctgtctcaaaaacaataaacagggctgggcatggtggctcacacctgtaatcccagcactttgggaggccaggagttccagaccagcctagccaacatggtgaaaccccttctctactaaaaataccaaaattagccaaggatggtggcacatgcttgtaatcccagctactcaggaggcagagacaggagaatctcttgaacttaggagcaggaggttgcagtgagtcgagactgcaccactgcactccagcctaggcaacagaggaagactctcatctcaaaaaaaataataaattttaaaaaataaataaatgaaataaaacaaagtagaaaccatcagggctttgaggttgggactgtggcatgccgaggggaggctgcaggggtcactgggaaacatcagggtccttcttcggttgcctgagcagcaccggtgtcctcaagccacattcagctgcatggcgccaatgctaagcagaaagaggaatcggaggcgccgagctggggcaagggtgggagccaagggagtggacacagcaggtagggccgggagacttgggtgaagcagtggaagccagagttctgaccctgctggagaagtgtcatgagggcctcagacaggttcacccactgagcaggaggtcttgcagcctgacccaccctgcatcctgttcaggccgggatcatctccttcctgggcttcttctggcccctttctgcatctcttcttgacaacatggcacaaatgaccagggttcttcccacctcaggtctttctgtggcttcctgctgcctgccatctatgttagccccaccgaccttaccctccacatcactgttttcctgtctgtcacccgtaagactttcagtgcctggggccttttcacatgggagtctgctttggagactgctccccttgcctgcttagcccccaccatgggccccaccccaaatcctccggacttctgagaattttcaatgttgagggcccaaccatgtgtttctttcttgcagtggctcacaggcccggggcattcaaagctgagctgtccaagctggtgattgtggccaaggcatcgcgcactgagctgtgaccagcagccctgttgcgggtggcaccttctcatctccggtgaagctgaaggggcctgtgtccctgaaagggccagcacatcactggttttctaggagggactcttaagttttctacctgggctgacgttgccttgtccggaggggcttgcagggtggctgaagccctggggcagagaacagagggtccagggccctcctggctcccaacagcttctcagttcccacttcctgctgagctcttctggactcaggatcgcagatccggggcacaaagagggtggggaacatgggggctatgctggggaaagcagccatgctccccccgacctccagccgagcatccttcatgagcctgcagaactgctttcctatgtttacccaggggacctcctttcagatgaactgggaagagatgaaatgttttttcatatttaaataaataagaacattaaaaagcaa

SEQ ID NO: 6 - DNA, 7380 bp, Mus musculus
gagcgcctgcgcattgccccggaagcaagatggcagcccccagcggaggcttctggactgcggtggtcctggcggccgcagcgctgaaattggccgccgctgtgtccgagcccaccaccgtgccatttgacgtgaggcccggaggggtcgtgcattcgttctcccaggacgtaggacccggggtacgtgctaagcctcctgcccgggaatcgatgcctatggacttccgatcaggtggtcctggagaccgggtggagtggcagaggcagggttacctgaagaagggacctggctgcctcgtctgtcttctgtcagtcactccctacctgcaaggcgacttccccttgcccatctcattgtagaaaagggaatactggttgataagagcagcggatctaggggactggctatacaggtcgcacgtgggttactcactgaacccggactagagcgggagcaccccccaagccttacacgagtccatcacctctcctttcagcgagccagcagtcagcctagcctctgcccgccccagcttggcttctgggacagcggtgtccctagtgaccatttcttgagaccattactatagtcctgtgctttctggcaggtttgtgttgtgggcaggaagcagtacaagcttgtatctaacaggagccccgagtactgacctcataggtgatgttctagggatgattgctgaaggccatttgaccttcctaggaaatcacagtgatttgagcaaagttctgagctcctgagaggagagagatctggtggcctctggagggtgtactgagggatggaacctagagtctcacatcggcagagccagggttctgccccgccctaggcaggggctccacccctgagccacatccctagctcctcattaggggattctaggtgggactccacccctgagtcaccctcccaacccctcactgctggattctggacaggctcaacccttgaccttctgggcaggggctgatgtatatctcagtcctttttgatctgttttacatcgggacagcaccttgctaagtcacccaggctagcttatgtcccagttcccagttctgcgtcagcctcctggataccagagatgatgtgcatacaggaccatgcccagtatgctagggtcttggttctgagtattgagatctttgctcttcagcctccttttatttctccttctacagaacaagtttacatgtacattcacctacgcttcccaaggagggaccaacgaggtaagttcctcagggtgtcacacgactggtgtcaagggaaagtggccagactcccgaggctctaaagtcccagtcccttgagcctgaaggacttctagttgccttggcttccgcctaatggaatcagcagcccacagtgctgatccctgactcttagtctatgtgtaatcagtgagccaccagagcctgggacagtcctcagggcagcccgtaagctggcaagagggccgatacctcagcagcacccagggcatcgaggtgaactgttatgaggttagctgcctgtcccacacacaagttagttcattcagtggaaatactggtgtatccagaaccttggacatgcatgttctgagcagagctttccataggagcaatgagtggaagtgtcttagtgtccacggatgggcaggggtacacagcacctcaacagttggaatatcacacacccgtgaacaagagcgttgcgtatggcatggagagggttaacagccctgctcgtggcatcgaatgaatgagtttattatgtacatagcaagtaaggaaaagaaatggagaggctacagacagcaaaaccatcttcagttaagtactcaatgagcagaaaccagcatgcagtgaccacagaggcccaaagtgggcatcggatctcctggaactggagttacagttgtgagccaccatgtggttgctgggaatcaaagtcaggtcctctgcaagagcagccagtgctataaccaccgagccatctctgccgatccttggttagtgaataagaagaaaccatctgaggaagcaagatggcagtcagctggagagcccatgggcaagcccctaggacctagctcccagcctatcctctcccctctaggcagggtttttaagcacgtgtatgtagttggatgtgtgggtgtatgcgcgtgtgtttggctctctgatgttagaggagcttgggttattgagtggttgtgagctgtcctgtgggtgctggggactgagcctgggtattctacttagactcagtactgctgtgatgagtcaccataaccgaagcaggctgggtggagggtgtggattcaggctgcacctccatatcactggcatcatagaaggaactcaagcagggcaggaacctggaggcaggagctgatgcagaggcccatgagggtgctgcttactggcttactcctcatggcttgcttaggctgctctcttagagaacccagagccaccagctcagagagggctccaccaacagtgggctaggctgatcactaattaagaaaatgtcctggaggctggtggaaagtctgattttatggaggtgttttctcaactaaggactcctcctctcctgtgactctagcttgagatgaaactgtccaggacgagcagcacctgttccccgaacctctctccagcagtctgtagttagctcctggagtggcttatgcctgcagttgtcgttgctgcccattaccaggctgggccatccctccctccaagaccatggagagagtgccttgagatcccacttccagaaccagtttcccagtgagctcaaggagagtgtgacagtgacgatttctattacaaggagagcggcactgacaccacccaagtctagagggacctcaaggatgctactcagtgagagacagagacacaagtcacgctgtgggattccattacatgaagtatccaggacaggagccctgagatagaaagtggatgcgctgggccagagagaggcttgagggaaggctcagcatggggctgccttttggactgatggaatgttctagatgcaaggtagtcatggctgcactgtggacttcacacggtgtgtgctttaatgggaatgatccaggggatggacattttctactttattctgtgagaggttttagggtttggctgtgccagagattaaatggctataagagggcccagtagcaagtaggatggatggcagctctcttgtggctgaggcaggaggatctggagttcaaggtcattctccactttacagagagatcaaggacacaagactgtcgctaaagaaaaagcaaggtgtggacccccttgagcagcgtttctcagatgggctattttcagactgcagaggctctgggaacagcagagccaggaattagcaggtctgtggcgtcggcaggaaccactcagagctggcccctggccttggcatgagtgcagttaggtctcagggcttcaccttggggagaatacacacagcttgctgcagggtccccttgtaattgacacaggtaggtcctggccatgagagccccccttgggaagccaggaagggcagtgatgggcgcgtctctgggtgagggctggcagcgagggccgcaaatactctgtgagaactggctagttgttttgtggattttgttcttgtttttgatttttatgttttaattttatgtgtatggtttttacttgcattaattatctgcaccagctgcatacctggtgcctgtggagaccgaaaaaggacttcaggttttctgtaactagttacagatggctgggagccactgtgtgggtgcaggtaacagaactctggtcctctgaaagagcagcctgtgctcttaggtacagggctgtctctccagccctggctggtttttggctttggttttggttgattgtgttggattgttggcttttgatagcccaggctggatccctacactttatgtagctgagactaaatcctgggtgtcctgagtgctaggtgctagggtgacagtcattccccattgcccatgtgcaaactacagacattctctactgtgcatgcgcacaccacggatccatgcaggacaattggaaacaggctgggtggtggtcctgcgaccctgaggaagctgtcagtgagtgtgttggcaccaaggcactggacggaggagggaagggaggaggagatggcagatagcaagcagggcctggggaatctagagtggagctagagccccgtgctgtagctgatccacagttcccaactgaatttagacagggtcttaactgttgccacttgttctcaagatgtcttacaggtaaaaaagtgactggatagtgccctccaccagcagggcagtgtctgtggctctagtgtaggatacagagcctaggagggaccatgtgtagccggccttttgaagacctgctaacacctgtccttgctcaccaagcctgtcctttctccccagcaatggcagatgagcctggggacaagtgaagacagccagcactttacctgtaccatctggaggtgagctgctgtgcacatgctgggctagaggctcgttctctctctccctctctctctccctctctctctccctctccctctccctctccctctctctctttctccagctactttgcatgcacacaccacagtccatgcaggacaagtaggctgggcagtggtcctgcgaccccaaggaagctccagtatgtgtgctggcaccacagcactgggcagaggagggaagagagaggaagatgtcagagagtaggcagggggctggggaatctggtcacacaacaggaagtcaccaaaacactgctgattctattgttcggttctggggaaacagaagccaggataccaggagctcaaggccagtcctggccacagagttagctgggggccattctgagcttcctcagatcctgtctcaaaaaccagtcttgaaacaaaaaaagaaagtatacagcaggcaatagccctgaccccagagctgtcatccgtcccttccccactccctgacctcagagctgtcacccctaccttccccactccctgaccccagagctgtcacccgtccctttcccactccctgaccccagagctgtcacccgtcccttccccactccctgaccccagagctgtcatccgtcccttccccactccctgaccccagagctgtcatccgtcccttccccactccctgaccccagagctgtcatccgtcccttccccactccctgaccccagagctgtcatccgtcccttccccactccctgaccccagagctgtcacccctcccttccccactccctgaccccagagctgtcatccgtccctttcccactccctgaccccagagctgtcatccgtcccttccccaatccctggcgcccacagaactacttcttacctctggctttggcatcctggagatttaccactatggagtcccacatcaccaggctctttccggggcctcagttgggtcctctccccctcccccattcctgtgctaactctgtctttccacaggccccaggggaaatcctacctctacttcacacagttcaaggctgagttgcgaggtgctgagatcgagtatgccatggcctacgtgagtgtcagtcacggggacccgaggacaaagggacctgcaaggccccagataccattcactgcaggggacagggccccgtgtcactcagccaccctgttgacccctagggctcccacccaagttgaggggatcagcgggaggcccactatgtgggtggttcctgcagagtggctggaggagccagggtggcctcggtcagctcctgtccctcaggcagtcaatgataggctcttcctcacagtaccagtgccctcagcagcctcaccaagtgtgtttctgggaaccaaggtgacagactgaggctgtccatcacctgtgatctgacccactgcttgggctctgctgggactactccctgggaataccacacccgccctagcaagcatgcctgactttaggccacagtgcccaggagacaaaactgaggggacccgaggggacttctctctccatgacgtgtgtctgctcaagtgttctgttttattttagtccaaagccgcatttgagagagagagtgatgtccccctgaaaagtgaggagtttgaagtgaccaagacagcaggtaggtgaaggtgggtcccagcagtggggggtacgccattacccagcaagagccatttggagagatctctgtggaggagggtcctcgtgcctgtgcactgcagatcagaaactgtctcgagaaagggctgcaggctaaattcaagctgcttgtgggatggactctgtgttgaggttgttccagccagtttggttcacagcatcctagtcaccagtactggtccactttctgttgctgtgacaacacacctgaggcaagctgctgcataaaacataaaggaccattcagtgcccaagtctagaggttcaagtgcaccagagcacctctgctagactctgtgaggacctggtgagggagacactggcagagccagggaagacagtccacatccccccatgaacttactcaaggcttcactggggaattctaggcagacgctccatgcctgcaacgcccccagctccctcacagggggctggggggagggggatcctcaacaaggactccaccctcaccatgcccactagccctctttgtaatttcccagtctcctttgtagttccccatgcagggcaggatctccttgtgtcacactgcccggctctgcagttttctattttcatccacctcgcgccctgtcagacctggcctctgtcggacctctgtcagggtttaattgtgcttgtgtcttgcagtgtctcacaggcctggggccttcaaagctgagctctccaagctggtgatcgtagccaaggcggcacgctcggagctgtgaccctcgcctgtcaagggccttcatgtccacgttcctcaggcacactgaccgggactacttgtctagggcactggttcccataggagctgccctgccctgcacaggtcacactgtgtcactccgcagaactctctgagcccggtcacctgttttgccagggaagatgcagggcatgtgcgggggtgggatggaaggacttcctggctttcctgaagtcaagatgtggtgtggtttcccctctgagccacagatgagtgtccccatcccaggaccactttctaaccccatccagggcagctccactcagaaggatgggaaaggatagaaaaaataaataaataagtagccaccttagtggtggctctgtggggtcaggactcaga

(19) United States
(12) Patent Application Publication
(10) Pub. No.: US 2023/0059560 A1
(43) Pub. Date: Feb. 23, 2023

(54) MYELOID-DERIVED GROWTH FACTOR FOR USE IN TREATING OR PREVENTING FIBROSIS, HYPERTROPHY OR HEART FAILURE

(71) Applicant: BOEHRINGER INGELHEIM INTERNATIONAL GMBH, lngelheim am Rhein (DE)

(72) Inventors: Kai Christoph WOLLERT, Hannover (DE); Mortimer KORF-KLINGEBIEL, Duderstadt (DE); Marc R. REBOLL, Laatzen (DE); Anton PEKCEC, lngelheim am Rhein (DE)

(21) Appl. No.: 17/759,056

(22) PCT Filed: Jan. 19, 2021
(86) PCT No.: PCT/EP2021/051079

§ 371 Date: Jul. 19, 2022

(30) Foreign Application Priority Data
Jan. 21, 2020 (EP) 20153008.6

(51) Int. Cl. A61K 38/18; A61P 9/04

(52) U.S. Cl. CPC A61K 38/18; A61P 9/04; A61K 48/00

(57) ABSTRACT

The present invention relates to the protein myeloid-derived growth factor (MYDGF) or nucleic acids encoding said protein for use in treating or preventing fibrosis and hypertrophy. The present invention also relates to the protein MYDGF or nucleic acids encoding said protein for use in treating heart failure. The present invention also relates to vectors comprising the nucleic acid, host cells expressing the nucleic acid, and methods for use in treating fibrosis and hypertrophy, and for use in treating heart failure.

### TECHNICAL FIELD OF THE INVENTION

The present invention relates to the protein myeloid-derived growth factor (MYDGF) or nucleic acids encoding said protein for use in treating or preventing fibrosis and hypertrophy. The present invention also relates to the protein MYDGF or nucleic acids encoding said protein for use in treating or preventing heart failure. The present invention also relates to vectors comprising the nucleic acid, host cells expressing the nucleic acid, and methods for use in treating fibrosis and hypertrophy, and for use in treating or preventing heart failure.

### BACKGROUND OF THE INVENTION

Myeloid-derived growth factor (MYDGF), also known as Factor 1, is a protein encoded in open reading frame 10 on human chromosome 19 (C19Orf10). The Protein was described in 2007 in a proteome-analysis of the so called fibroblast-like synoviocytes (FLS-cells) as a new secreted Factor in the synovium. A correlation between the secretion of the protein and inflammatory diseases of the joint has been supposed without any experimental or statistical evidence (Weiler et al., Arthritis Research and Therapy 2007, The identification and characterization of a novel protein, c19orf10, in the synovium). A corresponding patent application claims the protein as therapeutic agent for the treatment of joint and for the diagnosis of a tissue undergoing altered growth as well as monitoring changes in a tissue (US 2008/0004232 A1, Characterization of c19orf10, a novel synovial protein). Another scientific publication describes an enhanced expression of the protein in hepatocellar carcinoma cells (Sunagozaka et al., International Journal of Cancer, 2010, Identification of a secretory protein c19orf10 activated in hepatocellular carcinoma). Recombinant produced protein showed a proliferation enhancing effect on cultured hepatocellar carcinoma cells. It is noted that C19Orf10 has also been referred to as IL-25, IL-27 and IL-27W as it was originally considered an interleukin. However, the terms “IL-25” and “IL-27” have been used inconsistently in the art and have been used to designate a variety of different proteins. For example, US 2004/0185049 refers to a protein as IL-27 and discloses its use in modulating the immune response. This protein is structurally distinct from Factor 1 (compare Factor 1 amino acid sequence according to SEQ ID NO: 1 to the amino acid sequence of “IL-27” according to UniProt: Q8NEV9). Similarly, EP 2 130 547 A1 refers to a protein as IL-25 and discloses its use in treating inflammation. This protein has also been referred to in the art as IL-17E and is structurally distinct from Factor 1 (compare the amino acid sequence of Factor 1 according to SEQ ID NO: 1 to the amino acid sequence of “IL-25” according to UniProt: Q9H293).

WO 2014/111458 discloses Factor 1 for use in enhancing proliferation and inhibiting apoptosis of non-transformed tissue or non-transformed cells, in particular for use in treating acute myocardial infarction. Further disclosed are inhibitors of Factor 1 for medical use, in particular for use in treating or preventing a disease in which angiogenesis contributes to disease development or progression.

Korf-Klingebiel et al. (Nature Medicine, 2015, Vol. 21(2):140-149) report C19Orf10 to be secreted by bone marrow cells after myocardial infarction, which protein promotes cardiac myocyte survival and angiogenesis. The authors show that bone marrow-derived monocytes and macrophages produce this protein endogenously to protect and repair the heart after myocardial infarction, and propose the name myeloid-derived growth factor (MYDGF). In particular, treatment with recombinant Mydgf is reported to reduce scar size and contractile dysfunction after myocardial infarction.

Heart failure is a clinical syndrome with a poor prognosis that may develop in response to persistent hemodynamic overload, myocardial injury, or genetic mutations. Chronic inflammation contributes to the pathogenesis and progression of heart failure and has emerged as a therapeutic target (Adamo et al. Nat Rev Cardiol. 2020; 17:269-285). The relationship between heart failure and inflammation is bidirectional and involves crosstalk between the heart, immune system, and peripheral organs. In the myocardium, inflammatory cell-derived cytokines and growth factors promote contractile dysfunction and adverse left ventricular (LV) remodeling by acting on cardiac parenchymal and stromal cells (Bozkurt et al. Circulation. 1998; 97:1382-1391; Ismahil et al. Circ Res. 2014; 114:266-282; Sager et al. Circ Res. 2016; 119:853-864; Hulsmans et al. J Exp Med. 2018; 215:423-440; Bajpai et al. Nat Med. 2018; 24:1234-1245).

Acute pressure overload of the heart, as imposed by transverse aortic constriction (TAC) surgery in mice, elicits an inflammatory response involving the innate and adaptive immune systems (Martini et al. Circulation. 2019; 140:2089-2107). Signals emanating from stressed but viable cardiomyocytes trigger the inflammatory cascade (Suetomi et al. Circulation. 2018; 138:2530-2544). Within hours, cardiac expression levels of proinflammatory cytokines and chemokines increase (Baumgarten et al. Circulation. 2002; 105:2192-2197; Xia et al. Histochem Cell Biol. 2009; 131:471-481) and within a week most major immune cell subsets expand in size and/or display signs of activation in the pressure-overloaded heart (Xia 2009; Liao et al. Proc Natl Acad Sci USA. 2018; 115:E4661-E4669; Patel et al. JACC Basic Transl Sci. 2018; 3:230-244).

Fibrosis describes the formation of excess fibrous connective tissue in an organ or tissue in a reparative or reactive process such as a reactive, benign or pathological state. The connective tissue deposited during fibrosis can interfere with or inhibit the normal architecture and function of the underlying organ or tissue.

Hypertrophy describes the increase in the volume of an organ or tissue due to the enlargement of its component cells.

There remains a need for means and methods for treating hypertrophy and fibrosis. There also remains a need for means and methods for treating heart failure.

### SUMMARY OF THE INVENTION

The invention provides in a first aspect myeloid-derived growth factor (MYDGF) or a fragment or a variant thereof exhibiting the biological function of MYDGF, for use in treating or preventing fibrosis or hypertrophy.

According to one embodiment, the MYDGF protein or a fragment or a variant thereof exhibiting the biological function of MYDGF, is for use in treating or preventing heart failure. According to a preferred embodiment, the heart failure is chronic heart failure. According to a further embodiment, the heart failure or chronic heart failure is heart failure with preserved ejection fraction (HFpEF), heart failure with reduced ejection fraction (HFrEF), or heart failure with mid-range ejection fraction (HFmrEF).

According to a preferred embodiment, the MYDGF protein comprises SEQ ID NO: 1. Alternatively, the MYDGF protein comprises a fragment or variant of SEQ ID NO: 1, which exhibits the biological function of MYDGF, wherein the variant comprises an amino acid sequence with at least 85% amino acid sequence identity to SEQ ID NO: 1.

According to a preferred embodiment, the fibrosis is fibrosis of the heart, kidney, lung and/or liver. According to one embodiment, the fibrosis is an interstitial lung disease, preferably progressive fibrosing interstitial lung disease, and more preferably idiopathic pulmonary fibrosis.

According to a preferred embodiment, the hypertrophy is hypertrophy of cardiomyocytes.

According to a further aspect, the present invention provides a nucleic acid encoding the growth factor protein MYDGF or a fragment or a variant thereof exhibiting the biological function of MYDGF, for use in treating or preventing fibrosis or hypertrophy.

According to a further aspect, the present invention provides a nucleic acid encoding the growth factor protein MYDGF or a fragment or a variant thereof exhibiting the biological function of MYDGF, for use in treating heart failure.

According to one embodiment, the nucleic acid encodes an amino acid sequence having at least 85% sequence identity to SEQ ID NO:1.

According to a further aspect, the present invention provides a vector comprising the nucleic acid of the present invention for use in treating or preventing fibrosis or hypertrophy.

According to yet another aspect, the present invention provides a vector comprising the nucleic acid of the present invention for use in treating heart failure.

According to a further aspect, the present invention provides a host cell comprising the nucleic acid of the present invention or the vector of the present invention for use in treating or preventing fibrosis or hypertrophy. Preferably, the host cell expresses the nucleic acid.

According to yet another aspect, the present invention provides a pharmaceutical composition comprising the MYDGF protein, the nucleic acid, the vector or the host cell of the present invention and optionally a suitable pharmaceutical excipient, for use in treating or preventing fibrosis or hypertrophy.

According to yet another aspect, the present invention provides a pharmaceutical composition comprising the MYDGF protein, the nucleic acid, the vector or the host cell of the present invention and optionally a suitable pharmaceutical excipient, for use in improving heart function.

According to a preferred embodiment, the pharmaceutical composition for use is administered through the oral, intravenous, subcutaneous, intramucosal, intraarterial, intramuscular or intracoronary route. The administration is preferably through one or more bolus injection(s) and/or infusion(s).

According to a further aspect, the present invention provides a method of treating fibrosis. The method comprises administering to a patient in need thereof a therapeutically effective amount of MYDGF or fragment or variant thereof exhibiting the biological function of MYDGF.

According to one embodiment, the MYDGF comprises SEQ ID NO: 1 or a fragment or variant of SEQ ID NO: 1 and exhibiting the biological function of MYDGF. The variant comprises an amino acid sequence with at least 85% amino acid sequence identity to SEQ ID NO:1.

According to one embodiment, the fibrosis is fibrosis of the heart, kidney, lung and/or liver.

According to yet another embodiment, the MYDGF or fragment or variant thereof exhibiting the biological function of MYDGF is administered through one or more bolus injection(s) and/or infusion(s), preferably in a pharmaceutically accepted carrier.

According to a further aspect, the present invention provides a method of treating hypertrophy. The method comprises administering to a patient in need thereof a therapeutically effective amount of MYDGF or fragment or variant thereof exhibiting the biological function of MYDGF.

According to one embodiment, the MYDGF comprises SEQ ID NO: 1 or a fragment or variant of SEQ ID NO: 1 and exhibiting the biological function of MYDGF. The variant comprises an amino acid sequence with at least 85% amino acid sequence identity to SEQ ID NO:1.

According to a preferred embodiment, the hypertrophy is hypertrophy of cardiomyocytes.

According to yet another embodiment, the MYDGF or fragment or variant thereof exhibiting the biological function of MYDGF is administered through one or more bolus injection(s) and/or infusion(s), preferably in a pharmaceutically accepted carrier.

According to a further aspect, the present invention provides a method of treating or preventing heart failure, comprising administering to a patient in need thereof a therapeutically effective amount of growth factor MYDGF or fragment or variant thereof exhibiting the biological function of MYDGF. According to a preferred embodiment, the heart failure is chronic heart failure. According to a further preferred embodiment, the heart failure or chronic heart failure is HFpEF or HFrEF, preferably wherein the HFpEF is Stage C or Stage D HFpEF, or wherein the HFrEF is Stage C or Stage D HFrEF.

According to one embodiment, the MYDGF comprises SEQ ID NO: 1 or a fragment or variant of SEQ ID NO: 1 and exhibiting the biological function of MYDGF. The variant comprises an amino acid sequence with at least 85% amino acid sequence identity to SEQ ID NO:1.

According to yet another embodiment, the MYDGF or fragment or variant thereof exhibiting the biological function of MYDGF is administered through one or more bolus injection(s) and/or infusion(s), preferably in a pharmaceutically accepted carrier.

### BRIEF DESCRIPTION OF THE DRAWINGS

FIG. 1: Myeloid-derived growth factor inhibits transforming growth factor β1 (TGFβ1)-stimulated SMAD phosphorylation in lung fibroblasts from a patient with idiopathic pulmonary fibrosis. SMAD2 (Ser465/467) and SMAD3 (Ser423/425) phosphorylation (normalized to α-tubulin expression) in lung fibroblasts from a patient with idiopathic pulmonary fibrosis cultured in the absence or presence of TGFβ1 and/or MYDGF.

FIG. 2: Myeloid-derived growth factor inhibits transforming growth factor β1 (TGFβ1)-stimulated SMAD phosphorylation in left ventricular fibroblasts from a patient with terminal heart failure. SMAD2 (Ser465/467) and SMAD3 (Ser423/425) phosphorylation (normalized to the expression of unphosphorylated SMAD2/3) in left ventricular fibroblasts from a patient with terminal heart failure cultured in the absence or presence of TGFβ1 and/or Mydgf.

FIG. 3: Myeloid-derived growth factor inhibits transforming growth factor β1 (TGFβ1)-stimulated SMAD phosphorylation in left ventricular fibroblasts from a patient with terminal heart failure. SMAD2 (Ser465/467) and SMAD3 (Ser423/425) phosphorylation (normalized to the expression of unphosphorylated SMAD2/3) in left ventricular fibroblasts from a patient with terminal heart failure cultured in the absence or presence of TGFβ1 and/or MYDGF.

FIG. 4: Mouse myeloid-derived growth factor inhibits transforming growth factor β1 (Tgfß1)-stimulated Smad phosphorylation in mouse embryonic fibroblasts.

FIG. 5: MYDGF Attenuates Left Ventricular (LV) Remodeling During Pressure Overload. (A) Mydgf wild-type (WT) and knockout (KO) mice were subjected to transverse aortic constriction (TAC) or sham surgery (day 7). LV mass to tibia length ratio. Exemplary longitudinal tissue sections (day 7, scale bar, 1 mm) and summary data from 6-15 mice per group. ***P<0.001 vs same genotype sham (1-way ANOVA with Dunnett's post hoc test); #P<0.05, ##P<0.01 (2 independent sample t tests). (B) LV cardiomyocyte cross-sectional area. Exemplary tissue sections stained with wheat germ agglutinin (WGA; scale bar, 50 μm) and summary data from 3-7 mice per group. *P<0.05, ***P<0.001 vs same genotype sham (1-way ANOVA with Dunnett's post hoc test); #P<0.05, ###P<0.001 (2 independent sample t tests). (C) Size of isolated ventricular cardiomyocytes. Exemplary phase contrast microscopy images (day 7; scale bar, 100 μm) and summary data from 3-8 mice per group. ***P<0.001 vs same genotype sham (stat. test); #P<0.05 (stat. test). (D) Exemplary LV pressure-volume loops 7 days after sham or TAC surgery.

FIG. 6: Bone Marrow-Derived MYDGF Attenuates Left Ventricular (LV) Remodeling. (A-E) Bone marrow cells (BMCs) from Mydgf wild-type (WT) or knockout (KO) mice were transplanted into (→) KO or WT recipients. After bone marrow reconstitution, mice underwent transverse aortic constriction (TAC) surgery and were followed for 14 days. *P<0.05, **P<0.01 (2 independent sample t tests). (A) LV mass to tibia length ratio. 7-18 mice per group. (B) LV cardiomyocyte cross-sectional area. 4-5 mice per group. (C) LV isolectin B4 (IB4)⁺ endothelial cell density. 4-6 mice per group. (D) LV end-diastolic area (LVEDA) and LV end-systolic area (LVESA) as determined by echocardiography. 5-10 mice per group. LVEDA: P<0.05, WT→KO vs KO→KO. LVESA: P<0.01, WT→KO vs KO→KO (two independent sample t tests). (E) Fractional area change (FAC). Same animals as in (D). Circles represent individual mice. Horizontal bars are the means. *P<0.05, **P<0.01. (F) Experimental strategy used in (G-N) to overexpress MYDGF in inflammatory cells by lentiviral gene transfer (HSC denotes hematopoietic stem cell). (G) Exemplary immunoblots (of four) showing MYDGF and alpha-tubulin expression in BMCs and splenocytes from WT recipient mice 6 weeks after transplantation of lentivirally-transduced BMCs followed by doxycycline or no treatment for 1 week. (H) MYDGF plasma levels in WT recipient mice 6 weeks after transplantation of lentivirally-transduced BMCs followed by doxycycline or no treatment for 1 week. (I-M) All animals were treated with doxycycline. (I) Exemplary immunoblots showing LV MYDGF and GAPDH expression in WT recipient mice 6 weeks after transplantation of lentivirally-transduced BMCs followed by doxycycline or no treatment for 1 week. (J-L and N) *P<0.05, **P<0.01, ***P<0.001 vs same lentivirus sham; ##P<0.01, ###P<0.001 (2-way ANOVA with Tukey's post hoc test). (J) LV mass to tibia length ratio. 6 mice per group. (K) LV cardiomyocyte cross-sectional area. 5 mice per group. (L) IB4⁺ endothelial cell density. 6-7 mice per group. (M) LVEDA and LVESA. 6-8 mice per group. LVEDA and LVESA: P<0.001, Lenti.control TAC vs Lenti.control sham. LVEDA: P<0.05, TAC Lenti.MYDGF vs TAC Lenti.control. LVESA: P<0.01, TAC Lenti.MYDGF vs TAC Lenti.control. (N) FAC. Same animals as in (M).

FIG. 7: Cardiomyocyte Hypertrophy. Neonatal rat ventricular cardiomyocytes were stimulated for 24 hours with endothelin 1 (ET1, 100 nmol/L), angiotensin II (Ang II, 100 nmol/L), insulin-like growth factor (IGF, 50 ng/mL), and/or MYDGF (100 ng/mL, unless otherwise stated). (A) Exemplary immunofluorescence microscopy images. Scale bar, 50 mm. Summary data from 4-6 experiments. (B) Dose-response curve. Data from 4 experiments. The half-maximal inhibitory concentration (IC50) was calculated by 4 parameter logistic regression. Control denotes unstimulated cells' size. (C) Protein content. Data from 3 experiments. (D) Myh7 (beta myosin heavy chain), Nppa (natriuretic peptide type A), and Gapdh mRNA expression levels as determined by RT-qPCR. Data from 7-13 experiments. *P<0.05, ***P<0.001 vs unstimulated control; #P<0.05, ##P<0.01, ###P<0.001 (1-way ANOVA with Tukey's post hoc test).

FIG. 8: Phosphoproteome Analysis Identifies PIM1 as a Signaling Target of MYDGF. (A-D) Phosphoproteome analysis of neonatal rat ventricular cardiomyocytes (NRCMs) stimulated for 8 hours with endothelin 1 (ET1, 100 nmol/L) and/or MYDGF (100 ng/mL). (A) Flow chart illustrating the bottom-up approach to infer kinase activities from phosphoproteomic data and prior knowledge of kinase-substrate interactions. (B) Principal component analysis of the phosphoproteome data sets (4 biological replicates per condition). (C) Histogram illustrating phosphoproteomic changes. The red bars represent all phosphosites significantly regulated by ET1 vs unstimulated control (n=120, P<0.05, log₂ fold change greater than 1). The blue bars depict the regulation of these sites in ET1 plus MYDGF vs ET1 only-stimulated cells. (D) Substrate-based inference of kinase activities in ET1 plus MYDGF vs ET1 only-stimulated cells. (E) PIM1 protein expression and (F) kinase activity in NRCMs stimulated for 16 hours with ET1 and/or MYDGF. 6 experiments. *P<0.05, **P<0.01, ***P<0.001 (2 independent sample t tests). (G) NRCM size after stimulation with ET1, MYDGF, and/or SMI4a (10 μmol/L) for 24 hours. 3 experiments. *P<0.05 vs control, #P<0.05 (1-way ANOVA with Tukey's post hoc test). (H) NRCM size after transfection with scrambled (SCR) or PIM1 small interfering (si)RNAs and stimulation with ET1 and/or MYDGF for 24 hours. The exemplary immunoblots depict PIM1 and beta-actin expression after siRNA transfection. 4 experiments. ***P<0.001 vs control, ##P<0.01 (1-way ANOVA with Tukey's post hoc test).

FIG. 9: MYDGF Enhances SERCA2a Expression via PIM1. (A) Exemplary immunoblots and summary data showing PIM1, sarco/endoplasmic reticulum Ca²⁺ ATPase 2a (SERCA2a), and beta-actin expression in neonatal rat ventricular cardiomyocytes (NRCMs) stimulated with MYDGF (100 ng/mL). 4-5 experiments. *P<0.05 vs baseline (1-way ANOVA with Dunnett's post hoc test). (B) Exemplary immunoblots (of three) showing SERCA2a and beta-actin expression in NRCMs stimulated with MYDGF and/or SMI4a (10 μmol/L) for 16 hours. Where indicated, cells were first transfected with scrambled (SCR) or PIM1 small interfering (si)RNAs. (C) Exemplary immunoblots and summary data showing left ventricular (LV) SERCA2a and vinculin expression in Mydgf wild-type (WT) and knockout (KO) mice subjected to sham or transverse aortic constriction (TAC) surgery. 9 mice per group. ***P<0.001 vs same genotype sham (1-way ANOVA with Dunnett's post hoc test); #P<0.05 (2 independent sample t tests). (D) Exemplary immunoblots and summary data showing SERCA2a, PIM1, and alpha-tubulin expression in cardiomyocytes isolated from WT and KO mice 7 days after sham or TAC surgery. 5-6 mice per group. *P<0.05, ***P<0.001 vs same genotype sham; #P<0.05, ##P<0.01 (2 way ANOVA with Tukey's post hoc test). (E) Bone marrow cells from WT or KO mice were transplanted into (→) lethally irradiated KO or WT recipients. After bone marrow reconstitution, mice underwent TAC surgery and were followed for 14 days. Exemplary immunoblots and summary data showing LV SERCA2a, PIM1, and alpha-tubulin expression. 8 mice per group. *P<0.05 (2 independent sample t tests). (F) Exemplary immunoblots and summary data showing LV SERCA2a, PIM1, and beta-actin expression 7 days after TAC. Mice had been transplanted with Lenti.control or Lenti.MYDGF-transduced bone marrow cells and were treated with doxycycline starting 1 week prior to surgery. ***P<0.001 (2 independent sample t tests).

FIG. 10: MYDGF Protein Therapy. (A) Treatment regime. After transverse aortic constriction (TAC) surgery, mice received an intra-left ventricular (LV) cavity bolus injection of recombinant MYDGF (10 μg) followed by subcutaneous infusion for 3 (B), 7(C), or 42 (D-I) days (10 μg/day). TAC-operated control mice were treated with diluent only (bolus injection and infusion). (B) MYDGF plasma levels. 5-7 mice per group. ***P<0.001 (test). (C) Exemplary immunoblots and summary data showing LV sarco/endoplasmic reticulum Ca²⁺-ATPase 2a (SERCA2a) and alpha-tubulin expression. 5-7 mice per group. *P<0.05 (2 independent sample t test). (D) LV end-diastolic area (LVEDA) and LV end-systolic area (LVESA) as determined by serial echocardiography 7 and 42 days after TAC (16-22 mice per group) or 7 days after sham surgery (9 mice). LVEDA: P<0.01, TAC (control and MYDGF) vs sham at 28 days. LVESA: P<0.01, TAC (control and MYDGF) vs sham at 7 and 28 days (1-way ANOVA with Dunnett's post hoc test). LVEDA: P<0.01, MYDGF vs control at 28 days; LVESA: P<0.001 MYDGF vs control at 28 days (two independent sample t tests). (E) Fractional area change (FAC). Same animals as in (C). ***P<0.001 vs all TAC groups (1-way ANOVA with Dunnett's post hoc test); ##P<0.01, ###P<0.001 KO vs WT (2 independent sample t tests). (F) LV mass to tibia length ratio at 28 days. 6-12 mice per group. (G) LV cardiomyocyte cross-sectional area at 28 days. 6 mice per group. (H) Isolectin B4 (IB4)⁺ endothelial cell density in the left ventricle at 28 days. 6 mice per group. (E-G) *P<0.05, **P<0.01, ***P<0.001 vs sham (1-way ANOVA with Dunnett's post hoc test); #P<0.05, ###P<0.001 (2 independent sample t tests). (I) Cumulative survival after TAC in 27 control mice and 17 MYDGF-treated mice. *P=0.05 (log-rank test).

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

### Definitions

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

To practice the present invention, unless otherwise indicated, conventional methods of chemistry, biochemistry, cell biology, and recombinant DNA techniques are employed which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989). Furthermore, conventional methods of clinical cardiology are employed which are also explained in the literature in the field (cf, e.g., Braunwald's Heart Disease. A Textbook of Cardiovascular Medicine, 9^{th} Edition, P. Libby et al. eds., Saunders Elsevier Philadelphia, 2011).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. As used in this specification and the appended claims, the singular forms “a”, “an”, and “the” include plural referents, unless the content clearly dictates otherwise.

Nucleic acid molecules are understood as polymeric macromolecules made from nucleotide monomers. Nucleotide monomers are composed of a nucleobase, a five-carbon sugar (such as but not limited to ribose or 2′-deoxyribose), and one to three phosphate groups. Typically, a polynucleotide is formed through phosphodiester bonds between the individual nucleotide monomers. In the context of the present invention referred to nucleic acid molecules include but are not limited to ribonucleic acid (RNA) and deoxyribonucleic acid (DNA). The terms “polynucleotide” and “nucleic acid” are used interchangeably herein.

The term “open reading frame” (ORF) refers to a sequence of nucleotides, that can be translated into amino acids. Typically, such an ORF contains a start codon, a subsequent region usually having a length which is a multiple of 3 nucleotides, but does not contain a stop codon (TAG, TAA, TGA, UAG, UAA, or UGA) in the given reading frame. Typically, ORFs occur naturally or are constructed artificially, i.e. by gene-technological means. An ORF codes for a protein where the amino acids into which it can be translated form a peptide-linked chain.

The terms “protein” and “polypeptide” are used interchangeably herein and refer to any peptide-bond-linked chain of amino acids, regardless of length or post-translational modification. Proteins usable in the present invention (including protein derivatives, protein variants, protein fragments, protein segments, protein epitopes and protein domains) can be further modified by chemical modification. This means such a chemically modified polypeptide comprises other chemical groups than the 20 naturally occurring amino acids. Examples of such other chemical groups include without limitation glycosylated amino acids and phosphorylated amino acids. Chemical modifications of a polypeptide may provide advantageous properties as compared to the parent polypeptide, e.g. one or more of enhanced stability, increased biological half-life, or increased water solubility. Chemical modifications applicable to the variants usable in the present invention include without limitation: PEGylation, glycosylation of non-glycosylated parent polypeptides, covalent coupling to therapeutic small molecules, like glucagon-like peptide 1 agonists, including exenatide, albiglutide, taspoglutide, DPP4 inhibitors, incretin and liraglutide, or the modification of the glycosylation pattern present in the parent polypeptide. Such chemical modifications applicable to the variants usable in the present invention may occur co- or post-translational.

The term “amino acid” encompasses naturally occurring amino acids as well as amino acid derivatives. A hydrophobic non-aromatic amino acid in the context of the present invention, is preferably any amino acid which has a Kyte-Doolittle hydropathy index of higher than 0.5, more preferably of higher than 1.0, even more preferably of higher than 1.5 and is not aromatic. Preferably, a hydrophobic non-aromatic amino acid in the context of the present invention, is selected from the group consisting of the amino acids alanine (Kyte Doolittle hydropathy index 1.8), methionine (Kyte Doolittle hydropathy index 1.9), isoleucine (Kyte Doolittle hydropathy index 4.5), leucine Kyte Doolittle hydropathy index 3.8), and valine (Kyte Doolittle hydropathy index 4.2), or derivatives thereof having a Kyte Doolittle hydropathy index as defined above.

The term “variant” is used herein to refer to a polypeptide which differs in comparison to the polypeptide or fragment thereof from which it is derived by one or more changes in the amino acid sequence. The polypeptide from which a protein variant is derived is also known as the parent polypeptide. Likewise, the fragment from which a protein fragment variant is derived from is known as the parent fragment. Typically, a variant is constructed artificially, preferably by gene-technological means. Typically, the parent polypeptide is a wild-type protein or wild-type protein domain. Further, the variants usable in the present invention may also be derived from homologs, orthologs, or paralogs of the parent polypeptide or from artificially constructed variant, provided that the variant exhibits at least one biological activity of the parent polypeptide. The changes in the amino acid sequence may be amino acid exchanges, insertions, deletions, N-terminal truncations, or C-terminal truncations, or any combination of these changes, which may occur at one or several sites. In preferred embodiments, a variant usable in the present invention exhibits a total number of up to 23 (up to 1, 2, 3, 4, 5, 6, 7, 8, 9, 10, 11, 12, 13, 14, 15, 16, 17, 18, 19, 20, 21, 22, or 23) changes in the amino acid sequence (i.e. exchanges, insertions, deletions, N-terminal truncations, and/or C-terminal truncations). The amino acid exchanges may be conservative, and/or semi-conservative, and/or non-conservative. In preferred embodiments, a variant usable in the present invention differs from the protein or domain from which it is derived by up to 1, 2, 3, 4, 5, 6, 7, 8, 9, 10, 11, 12, 13, 14, 15, 16, 17, 18, 19, 20, 21, 22, or 23 amino acid exchanges, preferably conservative amino acid changes.

Typical substitutions are among the aliphatic amino acids, among the amino acids having aliphatic hydroxyl side chain, among the amino acids having acidic residues, among the amide derivatives, among the amino acids with basic residues, or the amino acids having aromatic residues. Typical semi-conservative and conservative substitutions are:

| Amino acid | Conservative substitution | Semi-conservative |
|---|---|---|
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C; |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C |

Changing from A, F, H, I, L, M, P, V, W or Y to C is semi-conservative if the new cysteine remains as a free thiol. Furthermore, the skilled person will appreciate that glycines at sterically demanding positions should not be substituted and that P should not be introduced into parts of the protein which have an alpha-helical or a beta-sheet structure.

Alternatively or additionally, a “variant” as used herein, can be characterized by a certain degree of sequence identity to the parent polypeptide or parent polynucleotide from which it is derived. More precisely, a protein variant in the context of the present invention exhibits at least 85% sequence identity to its parent polypeptide. Preferably, the polypeptide in question and the reference polypeptide exhibit the indicated sequence identity over a continuous stretch of 20, 30, 40, 45, 50, 60, 70, 80, 90, 100 or more amino acids or over the entire length of the reference polypeptide. Preferably, the polynucleotide in question and the reference polynucleotide exhibit the indicated sequence identity over a continuous stretch of 60, 90, 120, 135, 150, 180, 210, 240, 270, 300 or more nucleotides or over the entire length of the reference polypeptide.

The term “at least 85% sequence identity” is used throughout the specification with regards to polypeptide and polynucleotide sequence comparisons. This expression preferably refers to a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide.

Fragments of proteins comprise deletions of amino acids, which may be N-terminal truncations, C-terminal truncations or internal deletions or any combination of these. Such variants comprising N-terminal truncations, C-terminal truncations and/or internal deletions are referred to as “fragments” in the context of the present application. A fragment may be naturally occurring (e.g. splice variants) or it may be constructed artificially, preferably by gene-technological means. Preferably, a fragment (or deletion variant) has a deletion of up to 1, 2, 3, 4, 5, 6, 7, 8, 9, 10, 11, 12, 13, 14, 15, 16, 17, 18, 19, 20, 21, 22, or 23 amino acids at its N-terminus and/or at its C-terminus and/or internally as compared to the parent polypeptide, preferably at its N-terminus, at its N- and C-terminus, or at its C-terminus.

In case where two sequences are compared and the reference sequence is not specified in comparison to which the sequence identity percentage is to be calculated, the sequence identity is to be calculated with reference to the longer of the two sequences to be compared, if not specifically indicated otherwise.

The similarity of nucleotide and amino acid sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) or the CLUSTALW2 algorithm (Larkin M A, Blackshields G, Brown N P, Chenna R, McGettigan P A, McWilliam H, Valentin F, Wallace I M, Wilm A, Lopez R, Thompson J D, Gibson T J, Higgins D G. (2007). Clustal W and Clustal X version 2.0. Bioinformatics, 23, 2947-2948) which are available e.g. on http://npsa-pbil.ibcp.fr/cgi-bin/npsa_automat.pl?page=/NPSA/npsa_clustalw.html or on http://www.ebi.ac.uk/Tools/clustalw2/index.html. Preferably, the CLUSTALW2 algorithm on http://www.ebi.ac.uk/Tools/clustalw2/index.html is used wherein the parameters used are the default parameters as they are set on http://www.ebi.ac.uk/Tools/clustalw2/index.html: Alignment type=Slow, protein weight matrix=Gonnet, gap open=10, gap extension=0.1 for slow pairwise alignment options and protein weight matrix=Gonnet, gap open=10, gap extension=0.20, gap distances=5, No end gaps=no, Output options: format=Aln w/numbers, Order=aligned.

The grade of sequence identity (sequence matching) may be calculated using e.g. BLAST, BLAT or BlastZ (or BlastX). A similar algorithm is incorporated into the BLASTN and BLASTP programs of Altschul et al. (1990) J. Mol. Biol. 215: 403-410. BLAST protein searches are performed with the BLASTP program available e.g. on http://blast.ncbi.nlm.nih.gov/Blast.cgi?PROGRAM=blastp&BLAST_PROGRAMS=blastp&P AGE_TYPE=BlastSearch&SHOW_DEFAULTS=on&LINK_LOC=blasthome. Preferred algorithm parameters used are the default parameters as they are set on http://blast.ncbi.nlm.nih.gov/Blast.cgi?PROGRAM=blastp&BLAST_PROGRAMS=blastp&P AGE_TYPE=BlastSearch&SHOW_DEFAULTS=on&LINK_LOC=blasthome: Expect threshold=10, word size=3, max matches in a query range=0, matrix=BLOSUM62, gap costs=Existence: 11 Extension: 1, compositional adjustments=conditional compositional score matrix adjustment together with the database of non-redundant protein sequences (nr) to obtain amino acid sequences homologous to the Factor 1 and Factor 2 polypeptides.

To obtain gapped alignments for comparative purposes, Gapped BLAST is utilized as described in Altschul et al. (1997) Nucleic Acids Res. 25: 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:I54-I62) or Markov random fields. When percentages of sequence identity are referred to in the present application, these percentages are calculated in relation to the full length of the longer sequence, if not specifically indicated otherwise.

The term “host cell” as used herein refers to a cell that harbours a nucleic acid of the invention (e.g. in form of a plasmid or virus). Such host cell may either be a prokaryotic (e.g. a bacterial cell) or a eukaryotic cell (e.g. a fungal, plant or animal cell). The cell can be transformed or non-transformed. The cell can be an isolated cell for example in a cell culture or part of a tissue, which itself can be isolated or part of a more complex organization structure such as an organ or an individual.

The terms “myeloid-derived growth factor”, “MYDGF”, “Factor 1”, “MYDGF polypeptide or protein” or “Factor 1 polypeptide or protein” are used interchangeably and refer to the protein indicated in NCBI reference sequence NM_019107.3 (human homologue) as well as it mammalian homologues, in particular from mouse or rat. The amino acid sequence of the human homologue is encoded in open reading frame 10 on human chromosome 19 (C19Orf10). Preferably, MYDGF and Factor 1 protein refer to a protein, which comprises, essentially consists or consists of a core segment of human Factor 1 having an amino acid sequence according to SEQ ID NO: 1.

Whether or not a protein, variant or fragment exhibits the biological function of MYDGF can be determined by any one of the tests described in the examples below. According to the present invention, a peptide or protein exhibits the biological function of MYDGF if the results obtained with such peptide or protein compared to the results obtained with the MYDGF protein of the present invention shown in at least one of the examples presented herein below achieve at least 50%, at least 60%, at least 70%, at least 80%, at least 90%, at least 95% or 100% of the effect reported for MYDGF over the indicated controls.

As used herein, the terms “MYDGF” and “Mydgf” both denote myeloid-derived growth factor, wherein “MYDGF” is used in the present invention to refer to the human variant and “Mydgf” is used to refer to the mouse variant of myeloid-derived growth factor.

The term “improving heart function” as used herein means for example improving systolic and/or diastolic heart function, which can be assessed, for example, by echocardiography, cardiac magnetic resonance imaging, cardiac computed tomography, or ventricular angiography. For example, an increase in left ventricular dimensions and of systolic function of a heart is indicative of improving heart function and can be measured as e.g. as shown in example 10 below.

The term “fibrosis” describes the formation of fibrous tissue as a reparative or reactive process, as opposed to formation of fibrous tissue as a normal constituent of an organ or tissue. It has the same meaning as e.g. defined in Farlex Partner Medical Dictionary, in The American Heritage Medical Dictionary or in Pschyrembel Klinisches Wörterbuch, 261^{st} ed., 2007.

The term “hypertrophy” describes an abnormally high increase in the volume of an organ or tissue due to the enlargement of its component cells. In contrast to hyperproliferation, hypertrophy characterizes the increase in volume of a tissue or organ produced entirely by enlargement of existing cells, not by a high rate of proliferation of cells by rapid division. The term “hypertrophy” as used herein the same meaning as e.g. defined in Farlex Partner Medical Dictionary, in The American Heritage Medical Dictionary or in Pschyrembel Klinisches Wörterbuch, 261^{st} ed., 2007. Hypertrophy can be evaluated or measured by comparing e.g. the surface area, cross-sectional area or size of cells or a tissue afflicted with hypertrophy with the surface area, cross-sectional area or size of control cells or tissue not afflicted with hypertrophy, or by comparing the protein content of afflicted cells with a control. The hypertrophy to be treated according to the present invention is preferably a pathological hypertrophy, such as a hypertrophy caused by ET1 and/or Ang II, which are known to promote a pathological type of cardiac hypertrophy.

The term “interstitial lung disease” or “ILD” is to be understood as described in Lederer et al., New England Journal of Medicine, 2018, Vol. 378(19):1811-1823, or in van Cleemput, J. et al. Idiopathic Pulmonary Fibrosis for Cardiologists: Differential Diagnosis, Cardiovascular Comorbidities, and Patient Management; Adv Ther. 2019 February; 36(2):298-317.

The term “heart failure” and in the context of “treating and/or preventing heart failure” as used herein is to be understood as defined in the 2016 ESC Guidelines for the diagnosis and treatment of acute and chronic heart failure (European Heart Journal, 2016; Vol. 37(27):2129-2200) and includes chronic heart failure, heart failure with preserved ejection fraction (HFpEF), heart failure with reduced ejection fraction (HFrEF), heart failure with mid-range ejection fraction (HFmrEF). In the context of present invention, the term is also intended to refer to HFpEF or HFrEF, and in particular to Stage C or Stage D HFpEF and Stage C or Stage D HFrEF as described in 2017 ACC/AHH/HFSA Focused Update of the 2013 ACCF/AHA Guideline for the Management of Heart Failure (Journal of American College of Cardiology, 2017; Vol. 70(6):776-803). The description of the embodiments comprises further definitions and explanations of terms used throughout the application. These descriptions and definitions are valid for the whole application unless it is otherwise stated.

### Sequences

Sequences used in the present invention are listed below.
SEQ ID NO: 1 (amino acid sequence of human Factor 1, lacking the 31 aa N-terminal signal peptide):

VSEPTTVAFDVRPGGVVHSFSHNVGPGDKYTCMFTYASQGGTNEQWQMSL
GTSEDHQHFTCTIWRPQGKSYLYFTQFKAEVRGAEIEYAMAYSKAAFERE
SDVPLKTEEFEVTKTAVAHRPGAFKAELSKLVIVAKASRTEL

SEQ ID NO: 2 (amino acid sequence of the mouse homologue of Factor 1, lacking the 24 aa N-terminal signal peptide):

VSEPTTVPFDVRPGGVVHSFSQDVGPGNKFTCTFTYASQGGTNEQWQMSL
GTSEDSQHFTCTIWRPQGKSYLYFTQFKAELRGAEIEYAMAYSKAAFERE
SDVPLKSEEFEVTKTAVSHRPGAFKAELSKLVIVAKAARSEL

SEQ ID NO:3 (amino acid sequence of human Factor 1, including the N-terminal signal peptide (shown in bold and underlined); UniProtKB—Q969H8):

MAAPSGGWNGVGASLWAALLLGAVALRPAEAVSEPTTVAFDVRPGGVVH
SFSHNVGPGDKYTCMFTYASQGGTNEQWQMSLGTSEDHQHFTCTIWRPQG
KSYLYFTQFKAEVRGAEIEYAMAYSKAAFERESDVPLKTEEFEVTKTAVA
HRPGAFKAELSKLVIVAKASRTEL

SEQ ID NO: 4 (amino acid sequence of the mouse homologue of Factor 1, including the N-terminal signal peptide (shown in bold and underlined); UniProtKB—Q9CPT4):

MAAPSGGFWTAVVLAAAALKLAAAVSEPTTVPFDVRPGGVVHSFSQDVGP
GNKFTCTFTYASQGGTNEQWQMSLGTSEDSQHFTCTIWRPQGKSYLYFTQ
FKAELRGAEIEYAMAYSKAAFERESDVPLKSEEFEVTKTAVSHRPGAFKA
ELSKLVIVAKAARSEL

SEQ ID NO: 5 shows the nucleic acid sequence of human Factor 1 encoding MYDGF of SEQ ID NO: 3 (NCBI Gene ID: 56005).
SEQ ID NO: 6 shows the nucleic acid sequence of mouse Factor 1 encoding Mydgf of SEQ ID NO: 4 (NCBI Gene ID: 28106).

### EMBODIMENTS

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The present inventors show for the first time anti-fibrotic and anti-hypertrophic effects for MYDGF. The inventors particularly show that administration of MYDGF in a mouse model inhibits hypertrophy and fibrosis. These effects can be used inter alia for improving heart function. Therefore, in a first aspect, the invention provides the protein myeloid-derived growth factor (MYDGF) or a fragment or a variant thereof exhibiting the biological function of MYDGF, for use in treating or preventing fibrosis or hypertrophy.

In a second aspect, the invention provides the protein myeloid-derived growth factor (MYDGF) or a fragment or a variant thereof exhibiting the biological function of MYDGF, for use in treating or preventing heart failure. According to a preferred embodiment, the heart failure is chronic heart failure or acute heart failure, wherein acute heart failure does not include myocardial infarction. According to a preferred embodiment, the acute heart failure is acute pressure overload induced heart failure. Also provided is the MYDGF or a fragment or a variant thereof for this use, wherein the heart failure or chronic heart failure is heart failure with preserved ejection fraction (HFpEF), heart failure with reduced ejection fraction (HFrEF), heart failure with mid-range ejection fraction (HFmrEF). Without wishing to be bound by any theory, attenuating fibrosis and/or hypertrophy associated with heart failure and/or by improving heart function, heart failure is treated or prevented.

In a particularly preferred embodiment of the invention, the protein comprises the amino acid sequence SEQ ID NO: 1 or a fragment thereof. Preferably, the protein has at least 85%, 86%, 87%, 88%, 89%, 90%, 91%, 92%, 93%, 94%, 95%, 96%, 97%, 98%, or 99% identity to SEQ ID NO: 1.

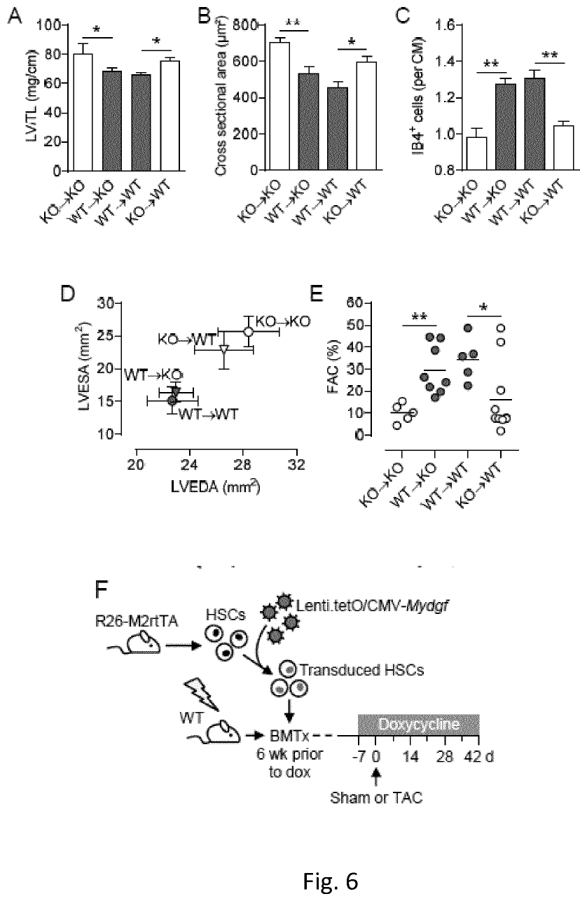

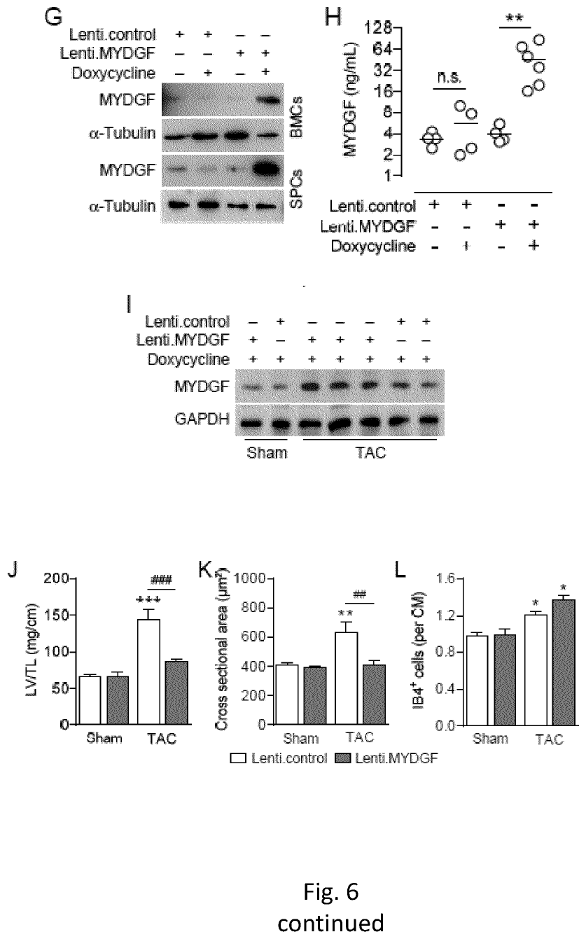

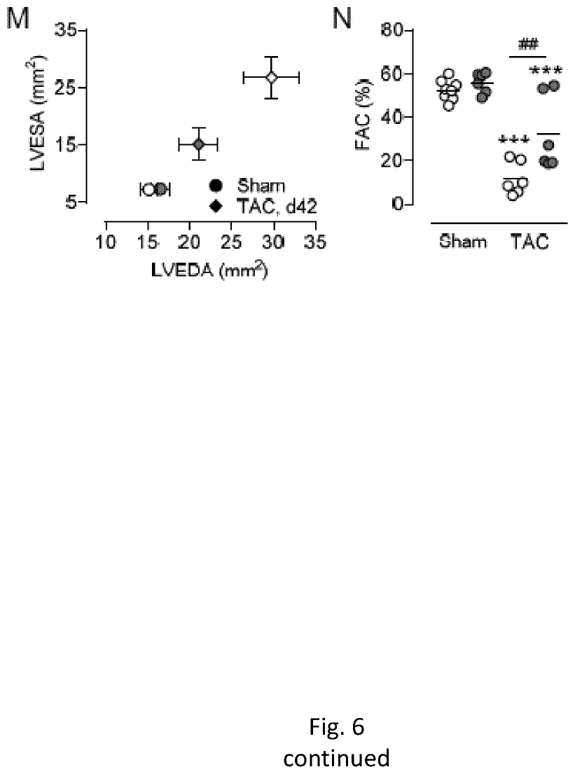

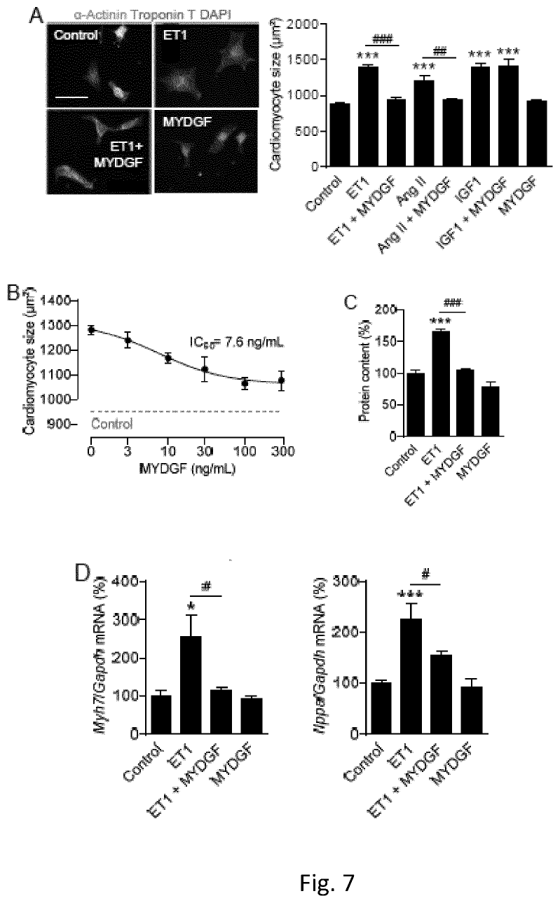

In a preferred embodiment of this aspect of the invention, the protein comprises the amino acid sequence SEQ ID NO: 1, a fragment or a variant thereof exhibiting the biological function of MYDGF, which has at least 85% sequence identity to SEQ ID NO: 1. A person skilled in the art is able to decide without undue burden, which positions in the parental polypeptide can be mutated to which extent and which positions have to be maintained to preserve the functionality of the polypeptide. Such information can, for example, be gained from homologues sequences which can be identified, aligned and analyzed by bioinformatic methods well known in the art. Such analyses are exemplarily described in example 7 and FIGS. 6 and 7 of WO 2014/111458. Mutations are preferably introduced in those regions of the protein, which are not fully conserved between species, preferably mammals. In a particularly preferred embodiment of the invention, the MYDGF protein comprises, essentially consists or consists of the amino acid sequence SEQ ID NO: 1 or a fragment or variant thereof exhibiting the biological function of MYDGF. Preferably, the protein has at least 85%, 86%, 87%, 88%, 89%, 90%, 91%, 92%, 93%, 94%, 95%, 96%, 97%, 98%, or 99% sequence identity to SEQ ID NO: 1.

N-terminal deletion variants are also encompassed, which may for example lack one or more amino acids from amino acid position 1 to 24 (based on SEQ ID NO: 1), i.e. from the N-terminally conserved region.

C-terminal deletion variants are also encompassed, which may for example lack one or more amino acids from amino acid position 114 to 142 (based on SEQ ID NO: 1).

On the other hand, amino acids can be added to the MYDGF protein. Such additions include additions at the N-terminus, at the C-terminus, within the amino acid sequence or combinations thereof. The protein of the first aspect of the present invention may thus further comprise additional amino acid sequences, e.g. for stabilizing or purifying the resulting protein. Examples of such amino acids are 6×His-tags, myc-tags, or FLAG-tags, which are well known in the art, and which may be present at any position in the protein, preferably at the N-terminus or the C-terminus. A particularly preferred additional sequence is a 6×His-tag. Preferably, said 6×His-tag is present on the C-terminus of the MYDGF protein. Depending on the expression system used and, if present, an additional amino acid such as a tag described above, one or more residual amino acids may remain on the N-terminus and/or the C-terminus of the protein. It is emphasized that in the MYDGF protein and Mydgf protein according to the present invention such artefacts may be present, as in shown e.g. in Ebenhoch R. et al., Nat Commun. 2019 Nov. 26; 10(1):5379, and Polten F. et al., Anal Chem. 2019 Jan. 15; 91(2):1302-1308.

In some cases it is preferred to mutate protease cleavage sites within the MYDGF protein of the first aspect of the present invention to stabilize the protein (see Segers et al. Circulation 2007, 2011). The skilled person knows how to determine potential proteolytic cleavage sites within a protein. For example, protein sequences can be submitted to websites providing such analysis as, e.g. http://web.expasy.org/peptide_cutter/or http://pmap.burnham.org/proteases. If the protein sequence according to SEQ ID NO: 1 is submitted to http://web.expasy.org/peptide_cutter/the following cleavage sites with lower frequency (less then 10) are determined:

TABLE 1

| | | Position |
|---|---|---|
| Protease | Frequency | (with reference to SEQ ID NO: 1) |
| Arg-C proteinase | 6 | 12 65 82 99 120 139 |
| Asp-N endopeptidase | 4 | 9 27 54 101 |
| Clostripain | 6 | 12 65 82 99 120 139 |
| LysN | 9 | 28 68 77 93 105 113 124 129 135 |
| Proline-endopeptidase | 3 | 13 66 121 |

These sites may be altered to remove the recognition/cleavage sequence of the respectively identified protease to increase the serum half-life of the protein.

MYDGF has been shown in the present invention to inhibit or prevent fibrosis, in particular fibrosis of the heart. Accordingly, the present invention provides the MYDGF protein or a fragment or a variant thereof exhibiting the biological function of MYDGF for use in treating or preventing fibrosis.

Treating or preventing fibrosis in the context of the present invention means e.g. reducing the amount of fibrotic tissue, or preventing or reducing the formation of fibrotic tissue. A reduction is preferably a reduction by at least 50%, at least 60%, at least 70%, at least 80% or at least 90% over a control tissue not treated with the active agent.

Without wishing to be bound by any theory, MYDGF inhibits transforming growth factor β, which is a universal profibrotic growth factor, thereby preventing and/or treating fibrosis.

MYDGF has also been shown in the present invention to inhibit or prevent hypertrophy, in particular hypertrophy of cardiomyocytes. Accordingly, the present invention provides the MYDGF protein or a fragment or a variant thereof exhibiting the biological function of MYDGF for use in treating or preventing hypertrophy. If hypertrophy of cardiomyocytes is treated or prevented, the cardiomyocytes are preferably cardiomyocytes of the left or right ventricle or cardiomyocytes of the heart's atria.

Treating and/or preventing hypertrophy and fibrosis of heart tissue and/or cells such as cardiomyocytes can also be used for improving function of the heart. Thus, the present invention also provides MYDGF for use in improving function of the heart. Heart function within the meaning of the present invention relates to systolic and/or diastolic heart function, which can be assessed, for example, by echocardiography, cardiac magnetic resonance imaging, cardiac computed tomography, or ventricular angiography. A preferred method for assessing heart function is high-resolution transthoracic 2D echocardiography (e.g. as described e.g. in Lang et al., Eur Heart J Cardiovasc Imaging, 2015; 16:233-270), for example in mice using a linear 30 MHz transducer (Vevo 3100, VisualSonics). In such experiment, left ventricular (LV) end-diastolic area (LVEDA) and left ventricular end-systolic area (LVESA) are determined from a long axis view. LVEDA (mm²) is a two-dimensional approximation of left ventricular end-diastolic volume; LVESA (mm²) is a two-dimensional approximation of left ventricular end-systolic volume. The fractional area change (FAC) is then calculated as a measure of systolic function, i.e. pumping or contractile function of the heart [(LVEDA−LVESA)/LVEDA]×100. Improving heart function thus means an improve in systolic and/or diastolic heart function, as measured by evaluating e.g. FAC, by at least 10% or more, such as 15%, 20%, 25%, 30%, 35%0, 40%, 45%, 50%, 55%, 60%, 65%, 70%, 75%, 80%, 85%, 90%, 95%, 100% or more than 100% such as 110%, 120%, 130%, 140%, 150%, 160%, 170%, 180%, 190%, 200% or more, compared to a heart function of the same heart before treatment with MYDGF according to the present invention.

The MYDGF protein may further comprise additional amino acid sequences, e.g. for stabilizing or purifying the resulting protein. For example, it is preferred to mutate protease cleavage sites within the MYDGF protein to stabilize the protein. Suitable proteolytic cleavage sites can be identified as described above.

The MYDGF protein or compositions comprising the protein can administered in vivo, ex vivo or in vitro, preferably in vivo.

The cells or tissue being fibrotic or hypertrophic preferably belong to or are derived from a defined system of the body of an individual selected from the group comprising the digestive, endocrine, excretory, immune, integumentary, muscular, nervous, reproductive, respiratory and skeletal system or combinations thereof. Alternatively, the cells or tissue being fibrotic or hypertrophic preferably belong to or are derived from a defined part or organ of the body of an individual selected from the group comprising: skin, bone, heart, cartilage, vessel, esophagus, stomach, intestine, gland, liver, kidney, lung, brain, and spleen. In a particularly preferred embodiment, the cells or tissue belong to or are derived from the heart.

The cells or tissue being fibrotic or hypertrophic can be damaged or diseased cells or tissue. Preferably, the damage or disease is caused through a genetic/inherited disease or an acquired disease resulting for example from ischemia, reperfusion injury, inflammation, infection, trauma, mechanical overload, intoxication or surgery. In a particularly preferred embodiment, the damage is caused by infarction, in particular myocardial infarction. In another particularly preferred embodiment, the damage is caused through a reperfusion injury.

In a particularly preferred embodiment, the cells or tissue inflicted with fibrosis are selected from the group consisting of heart, kidney, lung and liver cells or tissue, most preferably heart cells or tissue. The results shown in examples 15 to 18 based on cells from IPF patients highly suggest applicability on fibrosis in the context of ILD. The results shown in examples 21 and 22 based on embryonic fibroblasts suggest applicability also for other tissues such as kidney and liver fibrosis.

In a preferred embodiment, the fibrosis is an interstitial lung disease (ILD). According to one preferred embodiment, the ILD is progressive fibrosing interstitial lung disease, and more preferably idiopathic pulmonary fibrosis (IPF). Thus, according to one embodiment, the MYDGF protein or a fragment or a variant thereof exhibiting the biological function of MYDGF, is for use in preventing or treating interstitial lung diseases as defined in Lederer et al., New England Journal of Medicine, 2018, Vol. 378(19):1811-1823, and in van Cleemput, J. et al. Idiopathic Pulmonary Fibrosis for Cardiologists: Differential Diagnosis, Cardiovascular Comorbidities, and Patient Management. Adv Ther. 2019 February; 36(2):298-317. According to a further embodiment, the ILD is progressive fibrosing ILD (PF-ILD), and in particular idiopathic non-specific interstitial pneumonia (iNSIP), unclassifiable idiopathic interstitial pneumonia (unclassifiable IIP), idiopathic pneumonia with autoimmune features (IPAF), chronic hypersensitivity pneumonitis (CHP), environmental/occupational fibrosing lung diseases, systemic sclerosis interstitial lung disease (SSc-ILD), or rheumatoid arthritis interstitial lung disease (RA-ILD).

In a particularly preferred embodiment, the cells or tissue inflicted with hypertrophy are heart cells or tissue, more preferably cardiomyocytes.

In a further aspect, the present invention provides nucleic acids encoding the MYDGF protein or a fragment or a variant thereof exhibiting the biological function of MYDGF as described herein for use in treating or preventing fibrosis or hypertrophy. The present invention also provides nucleic acids encoding the MYDGF protein or a fragment or a variant thereof exhibiting the biological function of MYDGF as described herein for use in treating or preventing heart failure. The nucleic acids for use according to the present invention preferably encode an amino acid sequence having at least 85% sequence identity to SEQ ID NO:1.

Nucleic acid sequences can be optimized in an effort to enhance expression in a host cell. Parameters to be considered include C:G content, preferred codons, and the avoidance of inhibitory secondary structure. These Factors can be combined in different ways in an attempt to obtain nucleic acid sequences having enhanced expression in a particular host (cf. e.g. Donnelly et al., International Publication Number WO 97/47358). The ability of a particular sequence to have enhanced expression in a particular host involves some empirical experimentation. Such experimentation involves measuring expression of a prospective nucleic acid sequence and, if needed, altering the sequence. Starting with a particular amino acid sequence and the known degeneracy of the genetic code, a large number of different encoding nucleic acid sequences can be obtained. The degeneracy of the genetic code arises because almost all amino acids are encoded by different combinations of nucleotide triplets or “codons”. The translation of a particular codon into a particular amino acid is well known in the art (see, e.g., Lewin GENES IV, p. 119, Oxford University Press, 1990).

The nucleic acid for use according to the present invention may further comprise a transcriptional control element or expression control sequences positioned to control expression of the protein. Such a nucleic acid together with control elements is often termed as an expression system. The term “expression system” as used herein refers to a system designed to produce one or more gene products of interest. Typically, such system is designed “artificially”, i.e. by gene-technological means usable to produce the gene product of interest in vivo, in vitro or ex vivo. The term “expression system” further encompasses the expression of the gene product of interest comprising the transcription of the polynucleotides, mRNA splicing, translation into a polypeptide, co- and post-translational modification of a polypeptide or protein as well as the targeting of the protein to one or more compartments inside of the cell, the secretion from the cell and the uptake of the protein in the same or another cell. This general description refers to expression systems for the use in eukaryotic cells, tissues or organisms. Expression systems for prokaryotic systems may differ, wherein it is well known in the art, how an expression system for prokaryotic cells is constructed.

Regulatory elements present in a gene expression cassette generally include: (a) a promoter transcriptionally coupled to a nucleotide sequence encoding the polypeptide, (b) a 5′ ribosome binding site functionally coupled to the nucleotide sequence, (c) a terminator joined to the 3′ end of the nucleotide sequence, and (d) a 3′ polyadenylation signal functionally coupled to the nucleotide sequence. Additional regulatory elements useful for enhancing or regulating gene expression or polypeptide processing may also be present. Promoters are genetic elements that are recognized by an RNA polymerase and mediate transcription of downstream regions. Preferred promoters are strong promoters that provide for increased levels of transcription. Examples of strong promoters are the immediate early human cytomegalovirus promoter (CMV), and CMV with intron A (Chapman et al, Nucl. Acids Res. 19:3979-3986, 1991). Additional examples of promoters include naturally occurring promoters such as the EF1 alpha promoter, the murine CMV promoter, Rous sarcoma virus promoter, and SV40 early/late promoters and the [beta]-actin promoter; and artificial promoters such as a synthetic muscle specific promoter and a chimeric muscle-specific/CMV promoter (Li et al., Nat. Biotechnol. 17:241-245, 1999, Hagstrom et al., Blood 95:2536-2542, 2000).

The ribosome binding site is located at or near the initiation codon. Examples of preferred ribosome binding sites include CCACCAUGG, CCGCCAUGG, and ACCAUGG, where AUG is the initiation codon (Kozak, Cell 44:283-292, 1986). The polyadenylation signal is responsible for cleaving the transcribed RNA and the addition of a poly (A) tail to the RNA. The polyadenylation signal in higher eukaryotes contains an AAUAAA sequence about 11-30 nucleotides from the polyadenylation addition site. The AAUAAA sequence is involved in signalling RNA cleavage (Lewin, Genes IV, Oxford University Press, N Y, 1990). The poly (A) tail is important for the processing, export from the nucleus, translation and stability of the mRNA.

Polyadenylation signals that can be used as part of a gene expression cassette include the minimal rabbit [beta]-globin polyadenylation signal and the bovine growth hormone polyadenylation (BGH) (Xu et al., Gene 272:149-156, 2001, Post et al., U.S. Pat. No. 5,122,458).

Examples of additional regulatory elements useful for enhancing or regulating gene expression or polypeptide processing that may be present include an enhancer, a leader sequence and an operator. An enhancer region increases transcription. Examples of enhancer regions include the CMV enhancer and the SV40 enhancer (Hitt et al., Methods in Molecular Genetics 7:13-30, 1995, Xu, et al., Gene 272:149-156, 2001). An enhancer region can be associated with a promoter.

The expression of the MYDGF protein or variant thereof according to the present invention may be regulated. Such regulation can be accomplished in many steps of the gene expression. Possible regulation steps are, for example but not limited to, initiation of transcription, promoter clearance, elongation of transcription, splicing, export from the nucleus, mRNA stability, initiation of translation, translational efficiency, elongation of translation and protein folding. Other regulation steps, which influence the concentration of a MYDGF polypeptide inside a cell affect the half-life of the protein. Such a regulation step is, for example, the regulated degeneration of proteins. As the proteins of the invention comprise secreted proteins, the protein can be directed to a secretory pathway of the host cell. The efficiency of secretion regulates together with the regulatory steps referring to the expression and protein stability the concentration of the respective protein outside of the cell. Outside of the cell can refer to, for example but not limited to, a culture medium, a tissue, intracellular matrix or space or a body fluid such as blood or lymph.

The control of the regulatory steps mentioned above can be, for example, cell-type or tissue-type independent or cell-type or tissue-type specific. In a particularly preferred embodiment of the invention, the control of the regulatory steps is cell-type or tissue-type specific. Such a cell-type or tissue-type specific regulation is preferably accomplished through the regulation steps referring to the transcription of a nucleic acid. This transcriptional regulation can be accomplished through the use of cell-type or tissue-type specific promoter sequences. The result of this cell-type or tissue-type specific regulation can have different grades of specificity. This means, that the expression of a respective polypeptide is enhanced in the respective cell or tissue in comparison to other cell- or tissue-type or that the expression is limited to the respective cell- or tissue-type. Cell- or tissue-type specific promoter sequences are well known in the art and available for a broad range of cell- or tissue-types.

The expression is not necessarily cell-type or tissue-type specific but may depend from physiological conditions. Such conditions are for example an inflammation or a wound. Such a physiological condition-specific expression can also be accomplished through regulation at all above mentioned regulation steps. The preferred way of regulation for a physiological condition-specific expression is the transcriptional regulation. For this purpose a wound or inflammation specific promoter can be used. Respective promoters are, for example, natural occurring sequences, which can be, for example, derived from genes, which are specifically expressed during an immune reaction and/or the regeneration of wounded tissue. Another possibility is the use of artificial promoter sequences, which are, for example constructed through combination of two or more naturally occurring sequences.

The regulation can be cell-type or tissue-type specific and physiological condition-specific. Particularly, the expression can be a heart specific expression. Preferably, the expression is heart specific and/or wound specific.

Another possibility for a regulation of expression of the MYDGF protein or variant thereof according to the present invention is the conditional regulation of the gene expression. To accomplish conditional regulation, an operator sequence can be used. For example, the Tet operator sequence can be used to repress gene expression. The conditional regulation of gene expression by means of the Tet operator together with a Tet repressor is well known in the art and many respective systems have been established for a broad range of prokaryotic and eukaryotic organisms. A person of skill in the art knows how to choose a suitable system and adapt it to the special needs of the respective application.

In a particularly preferred embodiment the use of a nucleic acid according to the invention comprises the application to an individual, preferably an individual suffering from fibrosis or hypertrophy.

According to a further aspect, the present invention provides vectors comprising the nucleic acid or the expression system described herein for use in treating or preventing fibrosis or hypertrophy, or for use in treating heart failure.

As used herein, the term “vector” refers to a protein or a polynucleotide or a mixture thereof which is capable of being introduced or of introducing the proteins and/or nucleic acid comprised therein into a cell. In the context of the present invention it is preferred that the genes of interest encoded by the introduced polynucleotide are expressed within the host cell upon introduction of the vector or the vectors. Examples of suitable vectors include but are not limited to plasmid vectors, cosmid vectors, phage vectors such as lambda phage, filamentous phage vectors, viral vectors, viral like particles, and bacterial spores.

In a preferred embodiment of the invention, the vector is a viral vector. Suitable viral vectors include but are not limited to adenoviral vectors, adeno-associated viral (AAV) vectors, alphaviral vectors, herpes viral vectors, measles viral vectors, pox viral vectors, vesicular stomatitis viral vectors, retroviral vector and lentiviral vectors.

In a particularly preferred embodiment of the invention, the vector is an adenoviral or an adeno-associated viral (AAV) vector.

Nucleic acids encoding one or more MYDGF proteins or variants thereof according to the invention can be introduced into a host cell, a tissue or an individual using vectors suitable for therapeutic administration. Suitable vectors can preferably deliver nucleic acids into a target cell without causing an unacceptable side effect.

In a particularly preferred embodiment the use of a vector according to the invention, comprises the application to an individual in need thereof.

Vectors comprising nucleic acids encoding the MYDGF protein or fragments or variants thereof exhibiting the biological function of MYDGF described above are preferably for use in treating or preventing fibrosis of hypertrophy, or for use in treating heart failure.

According to a further aspect, the present invention provides a host cell comprising the vector as described herein and expressing the nucleic acid encoding the MYDGF protein or a fragment or a variant thereof exhibiting the biological function of MYDGF for use in treating or preventing fibrosis of hypertrophy, or for use in treating heart failure.

According to a further aspect, the present invention provides pharmaceutical compositions comprising the MYDGF protein or a fragment or a variant thereof exhibiting the biological function of MYDGF and optionally a suitable pharmaceutical excipient, for use in treating or preventing fibrosis or hypertrophy, or for use in treating heart failure.

The term “suitable pharmaceutical excipient”, as used herein, refers to a pharmacologically inactive substance such as but not limited to a diluent, excipient, surfactants, stabilizers, physiological buffer solutions or vehicles with which the therapeutically active ingredient is administered. “Pharmaceutical excipients” are also called “pharmaceutical carriers” and can be liquid or solid. Liquid carriers include but are not limited to sterile liquids, such as saline solutions in water and oils, including but not limited to those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. A saline solution is a preferred carrier when the pharmaceutical composition is administered intravenously. Examples of suitable pharmaceutical carriers are described in “Remington's Pharmaceutical Sciences” by E. W. Martin. In a preferred embodiment of the invention, the carrier is a suitable pharmaceutical excipient. Suitable pharmaceutical excipients comprise starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like. Such suitable pharmaceutical excipients are preferably pharmaceutically acceptable.

“Pharmaceutically acceptable” means approved by a regulatory agency of the Federal or a state government or listed in the U.S. Pharmacopeia or other generally recognized pharmacopeia for use in animals, and more particularly in humans.

The term “composition” is intended to include the formulation of the active compound with encapsulating material as a carrier providing a capsule in which the active component with or without other carriers, is surrounded by a carrier, which is thus in association with the active compound.

The term “active ingredient” refers to the substance in a pharmaceutical composition or formulation that is biologically active, i.e. that provides pharmaceutical value. In the context of the invention, the active ingredient is the MYDGF protein or the fragment or variant thereof exhibiting the biological function of MYDGF. A pharmaceutical composition may comprise one or more active ingredients which may act in conjunction with or independently of each other. The active ingredient can be formulated as neutral or salt forms. The salt form is preferably a pharmaceutically acceptable salt.

The term “pharmaceutically acceptable salt” refers to, for example but not limited to, a salt of the MYDGF polypeptides of the present invention including the fragments and variants thereof described herein. Suitable pharmaceutically acceptable salts include acid addition salts which may, for example, be formed by mixing a solution of the polypeptide of the present invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the peptide carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counter anions such as halide, hydroxide, carboxylate, sulfate, phosphate, nitrate, alkyl sulfonate and aryl sulfonate). Illustrative examples of pharmaceutically acceptable salts include, but are not limited to, acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (cf. e.g. S. M. Berge et al., “Pharmaceutical Salts”, J. Pharm. Sci., 66, pp. 1-19 (1977)).

According to one embodiment, the active ingredient is administered to a cell, a tissue or an individual in an effective amount. An “effective amount” is an amount of an active ingredient sufficient to achieve the intended purpose. The active ingredient may be a therapeutic agent. The effective amount of a given active ingredient will vary with parameters such as the nature of the ingredient, the route of administration, the size and species of the individual to receive the active ingredient, and the purpose of the administration. The effective amount in each individual case may be determined empirically by a skilled artisan according to established methods in the art. As used in the context of the invention, “administering” includes in vivo administration to an individual as well as administration directly to cells or tissue in vitro or ex vivo.

In a preferred embodiment of the invention, the pharmaceutical compositions are customized for the treatment of a disease or disorder. As used herein, “treat”, “treating” or “treatment” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); (e) limiting or preventing recurrence of symptoms in patients that were previously symptomatic for the disorder(s); (f) reduction of mortality after occurrence of a disease or a disorder; (g) healing; and (h) prophylaxis of a disease. The term “ameliorating” is also encompassed by the term “treating”. As used herein, “prevent”, “preventing”, “prevention”, or “prophylaxis” of a disease or disorder means preventing that such disease or disorder occurs in patient.

In a particularly preferred embodiment of the invention, a treatment with a pharmaceutical composition according to the invention comprises the treatment of an individual in need of such treatment.

The pharmaceutical composition contemplated by the present invention may be formulated in various ways well known to one of skill in the art. For example, the pharmaceutical composition of the present invention may be in liquid form such as in the form of solutions, emulsions, or suspensions. Preferably, the pharmaceutical composition of the present invention is formulated for parenteral administration, preferably for intravenous, intra-arterial, intramuscular, subcutaneous, transdermal, intrapulmonary, intraperitoneal intracoronary, intra-cardiac administration, or administration via mucous membranes, preferably for intravenous, subcutaneous, or intraperitoneal administration. A preparation for oral or anal administration is also possible. Preferably, the pharmaceutical composition of the present invention is in the form of a sterile aqueous solution which may contain other substances, for example, enough salts or glucose to make the solution isotonic with blood. The aqueous solutions should be suitably buffered (preferably to a pH of from 3 to 9, more preferably to a pH of from 5 to 7), if necessary. The pharmaceutical composition is preferably in unit dosage form. In such form the pharmaceutical composition is subdivided into unit doses containing appropriate quantities of the active component. The unit dosage form can be a packaged preparation, the package containing discrete quantities of pharmaceutical composition such as vials or ampoules.

The pharmaceutical composition is preferably administered through the intravenous, intra-arterial, intramuscular, subcutaneous, transdermal, intrapulmonary, intraperitoneal, intracoronary or intra-cardiac route, wherein other routes of administration known in the art are also comprised.

If the pharmaceutical composition is used as a treatment for an individual, the use of the pharmaceutical composition can replace the standard treatment for the respective disease or condition or can be administered additionally to the standard treatment. In the case of an additional use of the pharmaceutical composition, the pharmaceutical composition can be administered before, simultaneously or after a standard therapy.

It is further preferred that the pharmaceutical composition is administered once or more than once. This comprises 2, 3, 4, 5, 6, 7, 8, 9, 10, 11, 12, 13, 14, 15, 16, 17, 18, 19, 20, 25, 30, 35, 40, 45 or 50 times. The time span for the administration of the pharmaceutical is not limited. Preferably, the administration does not exceed 1, 2, 3, 4, 5, 6, 7 or 8 weeks.

A single dose of the pharmaceutical composition, can independently form the overall amount of administered doses, or the respective time span of administration can include administration as one or more bolus injection(s) and/or infusion(s).

According to a further aspect, the present invention provides a method of treating fibrosis, comprising administering to a patient in need thereof a therapeutically effective amount of MYDGF or fragment or variant thereof exhibiting the biological function of MYDGF. In said method, the MYDGF preferably comprises SEQ ID NO: 1, or a fragment or variant thereof exhibiting the biological function of MYDGF of SEQ ID NO: 1. In this respect, the fragment or variant comprises an amino acid sequence with at least 85% amino acid sequence identity to SEQ ID NO:1.

In a preferred embodiment of the method of the present invention, the fibrosis is fibrosis of the heart, kidney, lung and/or liver. According to a preferred embodiment, the fibrosis is an interstitial lung disease, more preferably progressive fibrosing interstitial lung disease, and most preferably idiopathic pulmonary fibrosis.

According to a preferred embodiment of this method of the present invention, the MYDGF protein or fragment or variant thereof exhibiting the biological function of MYDGF is administered through one or more bolus injection(s) and/or infusion(s), preferably in a pharmaceutically accepted carrier.

According to a further aspect, the present invention provides a method of treating hypertrophy, the method comprising administering to a patient in need thereof a therapeutically effective amount of MYDGF or fragment or variant thereof exhibiting the biological function of MYDGF. In said method, the MYDGF preferably comprises SEQ ID NO: 1, or a fragment or variant thereof exhibiting the biological function of MYDGF of SEQ ID NO: 1. In this respect, the fragment or variant comprises an amino acid sequence with at least 85% amino acid sequence identity to SEQ ID NO:1.

According to a preferred embodiment of the method of the present invention, the hypertrophy is hypertrophy of cardiomyocytes.

According to a preferred embodiment of the method of the present invention, the MYDGF protein or fragment or variant thereof exhibiting the biological function of MYDGF is administered through one or more bolus injection(s) and/or infusion(s), preferably in a pharmaceutically accepted carrier.

According to a preferred embodiment of this method of the present invention, the MYDGF protein or fragment or variant thereof exhibiting the biological function of MYDGF is administered through one or more bolus injection(s) and/or infusion(s), preferably in a pharmaceutically accepted carrier.

According to a further aspect, the present invention provides a method of treating or preventing heart failure, comprising administering to a patient in need thereof a therapeutically effective amount of growth factor MYDGF or fragment or variant thereof exhibiting the biological function of MYDGF, preferably wherein the heart failure is chronic heart failure. According to a preferred embodiment, in the method of treating heart failure, the heart failure or chronic heart failure is HFpEF or HFrEF, preferably wherein the HFpEF is Stage C or Stage D HFpEF, or wherein the HFrEF is Stage C or Stage D HFrEF. In this respect, the fragment or variant comprises an amino acid sequence with at least 85% amino acid sequence identity to SEQ ID NO:1.

According to a preferred embodiment of this method of the present invention, the MYDGF protein or fragment or variant thereof exhibiting the biological function of MYDGF is administered through one or more bolus injection(s) and/or infusion(s), preferably in a pharmaceutically accepted carrier.

### EXAMPLES

The Examples are designed to further illustrate the present invention and serve a better understanding. They are not to be construed as limiting the scope of the invention in any way.

### Materials and Methods Used in the Examples:

Unless stated otherwise, the following materials and methods were used in the examples.

Endothelin 1 (ET1) and angiotensin II (Ang II) were purchased from Sigma-Aldrich, murine insulin-like growth factor 1 (IGF1) from R&D Systems, and SMI4a from Selleckchem (catalog no. [#] S8005). Antibodies were bought from Abcam (sarco/endoplasmic reticulum Ca2+ATPase 2a [SERCA2a], polyclonal, #ab91032; alpha-tubulin, clone EPR13478(B)), Cell Signaling Technology (beta actin, clone 13E5; vinculin, clone E1E9V), Eurogentech (murine MYDGF, polyclonal, custom-made) (Korf-Klingebiel, 2015) and Thermo Fisher (PIM1, clone G.360.1).

Recombinant MYDGF. Recombinant murine MYDGF with a C-terminal 8×His-tag was produced in HEK293-6E cells cultured in animal origin-free, chemically defined, protein-free FreeStyle F17 expression medium (Gibco) (Karste et al. Methods Mol Biol. 2017; 1586:313-324). Transfection efficiency was assessed by cotransfecting a GFP-encoding control plasmid. Cell culture supernatants (6 L) were harvested by centrifugation and concentrated 15-fold by diafiltration against PBS (pH 7.4) containing 300 mmol/L NaCl (PBS/NaCl) in a Proflux M12 cross-flow ultrafiltration unit (Millipore) using 5 kDa Pellicon 2 filters (Millipore). The concentrate was filtered using 0.45/0.2 μm Sartobran 300 filter capsules (Sartorius) and sodium azide (0.05%) was added. MYDGF was captured by using an ÄKTA pure 25 M system (Cytiva) and 5 mL HisTrap excel column (Cytiva). The column was washed with PBS/NaCl until baseline UV signals were reached. Fractions eluted with imidazole (30 mmol/L) and containing the recombinant protein were pooled and concentrated using Vivaspin 20 (5,000 MWCO) ultrafiltration units (Sartorius). The protein was further purified by size exclusion chromatography (SEC) using a HiLoad 26/600 Superdex 75 column (GE Healthcare). MYDGF eluted in a single peak and was concentrated to 2 mg/mL using Vivaspin 20 (5,000 MWCO) ultrafiltration units. Protein concentrations were calculated by the molecular extinction coefficient (1 mg/mL=Abs_{280 nm} 0.95). The recombinant protein (total yield 5.95 mg/L) was characterized by SDS-PAGE and tryptic fingerprinting using mass spectrometry (Bruker). Protein samples were snap frozen in liquid nitrogen and stored at minus 80° C.

Mydgf Deficient Mice. Mice with a genetic deletion of Mydgf (Mydgf tm1Kcw, Mouse Genome Informatics ID: 5688472) have previously been described.18 The animals (BALB/c×C57BL/6J background) were backcrossed for 10 generations to the C57BL/6N strain and maintained on that background by heterozygous matings. Littermates were used in all experiments. Wild-type and targeted alleles were detected by genomic PCR (Korf-Klingebiel, 2015).

Mouse Surgery and Functional Assessment. All surgical procedures were approved by the authorities in Hannover, Germany (Niedersächsisches Landesamt für Verbraucherschutz und Lebensmittelsicherheit). Mice were housed in individually ventilated cages on a 12-hour light/dark cycle in the central animal facility of Hannover Medical School. Food and water were provided ad libitum. Transverse aortic constriction (TAC) surgery (Rockman et al. Proc Natl Acad Sci USA. 1991; 88:8277-8281) was performed in 8-10-week-old C57BL/6N mice. The animals were subcutaneously pretreated with 0.02 mg/kg atropine (B. Braun) and 200 mg/kg metamizole (Zentiva). Anesthesia was induced with 3-4% isoflurane (Baxter) in an induction chamber. After oral intubation, mice were mechanically ventilated (Harvard Apparatus, MiniVent Type 845), and anesthesia was maintained with 1.5-2% isoflurane. During surgery, mice were placed on a heating pad connected to a temperature controller (Föhr Medical Instruments) to keep rectal temperature at 37° C. A left anterolateral thoracotomy was performed under a surgical microscope. After separating the thymus and fat tissue from the aortic arch, a 6-0 silk suture was placed between the innominate and left carotid arteries and ligated against a 26 gauge blunt needle. After the knot was tied, the needle was removed. After wound closure, the mouse was disconnected from the ventilator and allowed to recover in a 32° C. incubator. In sham-operated control mice, the ligature around the aorta was not tied. Mice were subjected to a 3-week swim training protocol to induce physiological hypertrophy (Heineke, Methods Mol Biol. 2013; 963:279-301)

Transthoracic 2D echocardiography was performed with a linear 20-46 MHz transducer (MX400, Vevo 3100, VisualSonics) in mice sedated with 1-2% isoflurane via face mask. Pressure gradients across the aortic constriction site and right-to-left common carotid artery peak blood flow velocity (Vmax) ratios were determined immediately post TAC or sham surgery by Doppler sonography. LV end-diastolic area (LVEDA) and LV end-systolic area (LVESA) were recorded from the long-axis parasternal view. Fractional area change (%) was calculated as [(LVEDA−LVESA)/LVEDA]×100.

For LV pressure-volume measurements, mice were subcutaneously injected with 2 mg/kg butorphanol (Zoetis). Anesthesia was induced with 4% isoflurane. After oral intubation, mice were intraperitoneally injected with 0.8 mg/kg pancuronium (Actavis) and anesthesia was maintained with 2% isoflurane. A 1.4 F micromanometer-tipped conductance catheter (SPR-839, Millar Instruments) was inserted into the left ventricle via the right carotid artery. Steady-state pressure-volume loops were sampled at a rate of 1 kHz and analyzed with LabChart 7 Pro software (ADInstruments).

Bone Marrow Transplantation. Bone marrow cells (BMCs) were flushed from the femurs and tibias of 7-9-week-old Mydgf WT or KO donor mice. Erythrocytes were depleted by NH₄Cl lysis. 7-9-week-old Mydgf WT or KO recipient mice were lethally irradiated (9.5 Gy) and transplanted with 106 BMCs via the tail vein. After transplantation, mice were treated with ciprofloxacin (Bayer) for 3 weeks (100 mg/L in the drinking water). TAC surgery was performed 7-8 weeks after transplantation. Applying this protocol in a control experiment, CD45.2 recipient mice were lethally irradiated and transplanted with BMCs from congenic CD45.1 donor mice (B6.SJL-Ptprca Pepcb/BoyJ; Jackson Laboratory). After 8 weeks, more than 95% of blood leukocytes were CD45.1high as shown by flow cytometry using CD45.1 (BioLegend, clone A20) (dilution, 1:16) and CD45.2 (BD Biosciences, clone 104) (1:150) antibodies.23

Lentiviral Gene Transfer. A lentivirus encoding full-length murine MYDGF under the control of a tetO/CMV promoter and an empty control virus was generated in HEK 293T cells as previously described (Lachmann et al. Gene Ther. 2013; 20:298-307). To enable conditional MYDGF overexpression in bone marrow-derived inflammatory cells, hematopoietic stem cells (HSCs) were isolated from 7-9-week-old R26-M2rtTA mice expressing the reverse tetracycline-controlled transactivator (rtTA-M2) protein under the control of the ubiquitously active Rosa26 promoter. To this end, lineage negative (lin⁻) bone marrow cells were isolated by magnetic-activated cell sorting (MACS) using the Lineage Cell Depletion Kit from Miltenyi Biotec. Cells were expanded in serum-free StemSpan medium (Stem Cell Technologies) supplemented with cytokines (kit ligand, interleukin-3, interleukin-11, Flt3 ligand) and then transduced with lentiviral vector particle-containing HEK 293T cell supernatants in retronectin (TaKaRa Bio)-coated plates as previously described (Lachmann 2013; Kustikova et al. Exp Hematol. 2014; 42:505-515 e507). WT recipient mice were lethally irradiated (9.5 Gy) and transplanted with 1×10⁶ Lenti.control- or Lenti.MYDGF-transduced HSCs via the tail vein. After transplantation, mice were treated with ciprofloxacin for 3 weeks. Sham or TAC surgeries were performed 7 weeks after transplantation. To trigger MYDGF expression in lentivirally-transduced, HSC-derived inflammatory cells mice were treated with doxycycline (2 mg/mL in the drinking water) starting 1 week before surgery.

MYDGF Protein Therapy. An Alzet osmotic minipump (model 2004, pumping rate 0.25 μL/h for 28 days, filled with 10 μg MYDGF per 6 μL or diluent-only) was placed in a subcutaneous interscapular pocket immediately after TAC surgery. A single intraperitoneal bolus injection was applied thereafter (10 μg MYDGF or diluent-only).

Adenoviruses. Adenoviruses encoding SERCA2A or the red fluorescent protein DsRed were generated with the AdEasy adenoviral vector system (Agilent Technologies). Mice were injected with adenoviruses 5 days before TAC surgery (1×10¹⁰ plaque-forming units via the tail vein). Immediately after TAC, mice received a second adenovirus injection.

MYDGF-Targeted Liquid Chromatography/Multiple Reaction Monitoring-Mass Spectrometry. MYDGF concentrations in EDTA plasma samples from mice and patients were determined by targeted liquid chromatography/multiple reaction monitoring-mass spectrometry (LC/MRM-MS) (Polten et al. Anal Chem. 2019; 91:1302-1308).

Fluorescence-Activated Cell Sorting (FACS) and Flow Cytometry. Inflammatory cells were isolated from the left ventricle by enzymatic digestion and FACS (Hulsmans 2018; Korf-Klingebiel et al. Circ Res. 2019; 125:787-801). Left ventricles were digested for 30 min at 37° C. in PBS containing 1 mg/mL collagenase D (Roche), 2.4 mg/mL dispase (Gibco), and 100 U/mL DNase I (Sigma-Aldrich). Cell suspensions were filtered (40 μm cell strainer, Falcon), washed, and incubated for 5 min at 4° C. in PBS with 4% FCS, 2 mmol/L EDTA, and a purified mouse CD16/CD32 antibody (BD Biosciences, mouse BD Fc Block, clone 2.4G2) (1:55). Cells were then incubated for 20 min at 4° C. with the following antibodies: CD45R/B220-PE (clone RA3-6B2) (1:500), CD90.2/Thy-1.2-PE (clone 53-2.1) (1:2500), NK-1.1-PE (clone PK136) (1:500), CD49b/DX5-PE (clone DX5) (1:500), Ly6G-PE (clone 1A8) (1:500), and CD11b-Alexa Fluor 700 (clone M1/70) (1:50) from BD Biosciences; Ly6C-APC (clone 1G7.G10) (1:8) from Miltenyi Biotec; and CD45-Brilliant Violet 570 (clone 30-F11) (1:33), F4/80-FITC (clone BM8) (1:33), CD3-PE/Cy7 (clone 17A2) (1:33), and CD19-PerCP/Cy5.5 (clone 6D5) (1:33) from BioLegend. After washing, the cells were sorted on a FACSAria IIu instrument (Becton Dickinson). Monocytes were identified as CD45high CD11bhigh (CD45R/B220, CD90.2/Thy-1.2, NK 1.1, CD49b/DX5, Ly6G)low F4/80low Ly6Chigh; macrophages as CD45high CD11bhigh (CD45R/B220, CD90.2/Thy-1.2, NK 1.1, CD49b/DX5, Ly6G)low F4/80high or low Ly6Clow; neutrophils as CD45high CD11bhigh (CD45R/B220, CD90.2/Thy-1.2, NK 1.1, CD49b/DX5, Ly6G)high; T cells as CD45high CD11blow (CD45R/B220, CD90.2/Thy-1.2, NK 1.1, CD49b/DX5, Ly6G)high CD3high CD19low; and B cells as CD45high CD11blow (CD45R/B220, CD90.2/Thy-1.2, NK 1.1, CD49b/DX5, Ly6G)high CD3low CD19high. For flow cytometry, inflammatory cells were incubated with labeled antibodies as described above. Cells were then added to TruCOUNT tubes (BD Biosciences), counted on an LSR II flow cytometer (Becton Dickinson), and analyzed with FlowJo v10.6 software.

Endothelial Cell and Fibroblast Isolation by MACS. LV myocardium was enzymatically digested with collagenase I (Worthington) and DNase I (Sigma-Aldrich). Cell suspensions were filtered (30-μm cell strainer, Falcon), washed, incubated with CD45 MicroBeads, and applied to LD columns. The flow-through CD45low cell fraction was washed, incubated with CD146 MicroBeads, and applied to LD columns. Endothelial cells (CD45low CD146high) were eluted from the columns and used for RNA isolation (RNeasy kit, Qiagen). The flow-through CD45low CD146low cell fraction was washed, incubated with Feeder Removal MicroBeads, and applied to LS columns. Fibroblasts were eluted from the columns and used for RNA isolation (all reagents and equipment from Miltenyi Biotec).

Tissue Collection and Analyses. Mice were sacrificed at different time points after TAC or sham surgery and the left ventricles were removed. RNA was isolated using RNeasy kits (Qiagen). Protein lysates were prepared in RIPA buffer. Midventricular slices were embedded in OCT compound (Tissue Tek), snap-frozen in liquid nitrogen, and stored at minus 80° C. 6-μm cryosections were prepared. Sections were stained with rhodamine-conjugated wheat germ agglutinin (WGA, Vector Laboratories) to highlight cardiomyocyte borders. The circumferences of 200-400 myocytes were traced and digitized to calculate mean cross-sectional area. Sections were stained with fluorescein-conjugated GSL I isolectin B4 (IB4, Vector Laboratories) to visualize capillaries. Images were acquired by fluorescence microscopy (Axio Observer.Z1). MYDGF was visualized by confocal fluorescence microscopy in 6 μm cryosections (Leica DM IRB with a TCS SP2 AOBS scan head) after staining with the polyclonal antibody from Eurogentec (1:100)18 and an FITC-labeled polyclonal secondary antibody (Invitrogen, #A24532) (1:200). Sections were costained with a CD11b antibody (Invitrogen, clone M1/70) (1:200) and a Cy3-labeled polyclonal secondary antibody (Jackson ImmunoResearch, #712-165-153) (1:200). In pilot experiments, all secondary antibodies were found to yield low background signals. Sections were stained with Sirius red (Sigma-Aldrich) to quantify interstitial collagen volume fraction using light microscopy.

Cardiomyocyte Isolation and Culture. Adult mouse ventricular cardiomyocytes (AMCMs) were isolated by enzymatic digestion using a protocol from the Alliance for Cellular Signaling (http://www.signaling-gateway.org/data/cgi-bin/Protocols.cgi?cat=0). AMCMs were plated on laminin-coated 6 well plates in medium 199 (Sigma-Aldrich) supplemented with 5% FCS and 10 mmol/L 2,3-butanedione monoxime (Sigma-Aldrich) and cell length and width were determined by phase contrast microscopy and a digital image analyzer (AxioVision, Zeiss). Neonatal rat ventricular cardiomyocytes (NRCMs) were isolated from 1-3-day-old Sprague-Dawley rats by Percoll density gradient centrifugation (Shubeita et al. A paracrine mechanism for myocardial cell hypertrophy. J Biol Chem. 1990; 265:20555-20562). NRCMs were plated overnight on gelatin-coated culture dishes in DMEM (Capricorn Scientific, 4 parts) and medium 199 (1 part), supplemented with 5% horse serum, 2.5% FCS, glutamine, and antibiotics. Cells were then switched to DMEM and medium 199 supplemented only with glutamine and antibiotics and stimulated with various agents for 24 hours. NRCM surface area was determined by planimetry, protein content by the Bradford assay (Bio-Rad). NRCMs were transfected with siRNAs (50 pmol/106 cells) using the Lipofectamine RNAiMAX reagent and siRNAs from Thermo Fisher (PIM1: #4390771, ID: s128205; scrambled: #4390843, Silencer Select siRNA-negative control).

Quantitative Reverse Transcription-Polymerase Chain Reaction (RT-qPCR). Total RNA was isolated from cells and tissues with RNeasy RNA isolation kits (Qiagen) followed by reverse transcription into cDNA (SuperScript III reverse transcriptase, Thermo Fisher); mRNA concentrations were determined with TaqMan or SYBR green assays (annealing, 60° C. for 1 min; extension, 72° C. for 1 min) (all reagents from Thermo Fisher). Data were analyzed using standard curves (TaqMan) or relative quantification (SYBR green).

Sample Preparation for Proteomics and Phosphoproteomics. After stimulation, NRCMs (2×10⁶ cells per replicate) were washed twice with ice-cold PBS; solubilized with 400 μL ice-cold RIPA buffer containing 24 mmol/L Tris-HCl (pH 7.6), 150 mmol/L NaCl, 1% NP-40, 1% sodium desoxycholate, 0.1% sodium dodecyl sulfate, cOmplete mini protease inhibitor cocktail (Roche), and PhosSTOP (Roche); and frozen overnight at −80° C. After thawing, samples were dispersed on ice with an IKA Ultra-Turrax and centrifuged at 14,000 g at 4° C. for 5 min. The supernatant was transferred to a new reaction tube and the protein concentration was measured with the DC protein assay (Bio-Rad). For proteomics, 50 μg protein was processed using SDS-PAGE and in-gel trypsin digestion as previously described (Polten et al. Anal Chem. 2019; 91:1302-1308). Specifically, each gel lane was cut into six pieces that were separately digested to generate six peptide samples. For phosphoproteomics, a modified filter-aided sample preparation protocol was employed (Nel et al. J Proteome Res. 2015; 14:1637-1642). Briefly, 300 μg protein was combined with 200 μL urea buffer (8 mol/L urea, 0.1 mol/L Tris-HCl [pH 9.5]) and loaded onto a 10 kDa Amicon Ultra-0.5 mL centrifugal filter (Merck). The filter membrane was washed twice with 200 μL urea buffer and free cysteines were carbamidomethylated by adding 100 μL 50 mmol/L iodoacetamide in urea buffer for 20 min in the dark. Thereafter, the filter membrane was washed twice with 100 μL urea buffer and twice with 100 μL 40 mmol/L NH₄HCO₃. The tryptic digest was carried out in 120 μL 40 mmol/L NH₄HCO₃ containing 7 μg mass spectrometry-grade trypsin (Serva). After overnight digestion at 37° C., peptides were eluted twice with 40 μL 40 mmol/L NH₄HCO₃. The combined flow-through was acidified by adding 12 μL 10% trifluoroacetic acid (TFA) and then dried by vacuum centrifugation. Phosphorylated peptides were enriched using the Pierce Fe-NTA phosphopeptide enrichment and Pierce TiO₂ phosphopeptide enrichment kits from Thermo Scientific, following the manufacturer's protocols. Briefly, after equilibrating the Fe-NTA column, the dried digested peptides were dissolved in 200 μL binding buffer, applied to the column, and incubated for 30 min at room temperature. After centrifugation, the flow-through was collected. After two washing steps with 100 μL washing buffer A and two washing steps with 200 μL washing buffer B, the flow-through and wash fractions were combined, dried by vacuum centrifugation, and loaded onto a TiO₂ phosphopeptide enrichment spin tip. After washing, phosphopeptides bound to the spin tip were eluted and dried by vacuum centrifugation. Phosphopeptides bound to the Fe-NTA column were eluted twice with 100 μL elution buffer. Eluate fractions were combined and dried by vacuum centrifugation. Phosphopeptide eluates from the Fe-NTA- and TiO₂-based enrichment steps were desalted using Pierce graphite spin columns (Thermo Fisher).

Liquid Chromatography-Tandem Mass Spectrometry (LC-MS/MS) and Data Processing. The six peptide and two phosphopeptide samples per replicate were each reconstituted in 30 μL high-performance liquid chromatography loading buffer (2% acetonitrile, 0.1% TFA) and separately analyzed using an UltiMate 3000 RSLCnano system (Thermo Scientific) connected to an LTQ Orbitrap Velos mass spectrometer (Thermo Scientific) as previously described (Junemann et al. Front Microbiol. 2018; 9:3083). Generated spectra were analyzed with MaxQuant software (version 1.6.1.0) applying the integrated Andromeda peptide search engine against the rat UniProt Knowledgebase (Tyanova et al. Nat Protoc. 2016; 11:2301-2319; UniProt Consortium. UniProt: a worldwide hub of protein knowledge. Nucleic Acids Res. 2019; 47:D506-D515). Propionamidation (C) (proteome analysis) or carbamidomethylation (C) (phosphoproteome analysis) were set as fixed modifications; phosphorylation (S/T/Y), oxidation (M), deamidation (N/Q), and N-terminal acetylation as variable modifications. Peptide lengths above six amino acids and up to two missed trypsin cleavages were allowed. The false discovery rate (FDR) threshold was set to 0.01 both on peptide and protein levels and the match-between-runs algorithm was applied. Potential contaminants and reverse database hits were excluded. Missing values were imputed based on the normal distribution of all measured log₂-transformed intensities using Perseus software (version 1.6.1.3) applying a width of 0.3 and a down shift of 1.8, separately for each replicate (Tyanova et al. Nat Methods. 2016; 13:731-740). For phosphoproteome analysis, only phosphorylation sites with a localization probability greater than 0.75 were considered. Phosphorylation sites not detectable in all four replicates from at least one experimental condition were filtered out. Phosphorylation site intensities were normalized on the corresponding protein abundance. Principal component analysis was based on all phosphorylation sites with ANOVA P values <0.05 as calculated by Perseus. Clusters in principal component space were delineated based on linear regression of replicate values. Unsupervised hierarchical clustering was performed with ComplexHeatmap package for R using median-adjusted phosphorylation site intensities and the Euclidean distance metric for row and column trees (Gu et al. Bioinformatics. 2016; 32:2847-2849). For pairwise comparisons, differences in phosphorylation intensities were normalized to the corresponding differences in protein abundance. Missing quantification values in the proteome in at least one condition or non-significantly regulated protein (2-sided unpaired t test P value >0.05) were considered as a protein abundance ratio of 1. Linear kinase motif enrichment analysis was performed using Perseus and phosphorylation site annotations from the PhosphoSitePlus database (Hornbeck et al. Nucleic Acids Res. 2015; 43:D512-520; Hogrebe et al. Nat Commun. 2018; 9:1045). Kinase-substrate relations were predicted based on the sequence recognition motif around the measured phosphorylation sites. Significantly regulated phosphorylation sites' kinase substrate motifs were categorically enriched with Fisher's exact test. For each significantly regulated kinase (Benjamini-Hochberg FDR<0.02), all potential targets were extracted (kinase-substrate recognition motif positive, localization probability >0.75, 2-tailed unpaired t test P value <0.05) and the arithmetic mean regulation was determined (Hogrebe 2018).

PIM Kinase Activity Assay. PIM kinase activity was measured with the PIM kinase enzyme system and ADP-Glo kinase assay (Promega, #V4032).

Measuring Intracellular Ca²⁺ Concentration. AMCMs were plated on laminin-coated glass coverslips in medium 199 supplemented with 5% FCS and 10 mmol/L 2,3-butanedione monoxime. After 3 hours, cells were switched to medium 199, loaded with 1.5 μmol/L fura-2, AM (Invitrogen, #F1221) for 20 min at 37° C., washed twice for 15 min, and transferred to a custom-made perfusion chamber. The cells were electrically stimulated with a MyoPacer EP stimulator (IonOptix) under constant recirculation with an isotonic electrolyte solution containing (in mmol/L) NaCl 117, KCl 5.7, NaH₂PO₄ 1.2, CaCl₂ 1.25, MgSO₄ 0.66, glucose 10, sodium pyruvate 5, creatine 10, and HEPES 20 (pH 7.4) (Mutig et al. Mol Immunol. 2013; 56:720-728). Rod-shaped, quiescent cardiomyocytes with well-defined striations that reacted to stimulation (1 Hz, 15 V, 4 ms impulse duration) were randomly selected. Single cell Ca²⁺-transients were recorded by measuring fluorescence emitted at 510 nm after excitation with alternating wavelengths of 340 and 380 nm using a dual excitation fluorescence photomultiplier system (IonOptix) as previously described (Mutig 2013; Dobson et al. Am J Physiol Heart Circ Physiol. 2008; 295:H2364-2372). Average background fluorescence was recorded separately from a group of 10 cells not loaded with fura-2, AM and subtracted before calculating 340 nm/380 nm fluorescence ratio (R). Data from 20 calcium transients per cell were averaged. Fura-2, AM ratio amplitude, maximum velocity of fura-2, AM ratio increase, and time constant (τ) of the fura-2, AM ratio decay were analyzed using IonWizard 6.5.

Single-Cell Sarcomere Contraction and Relaxation Analysis. AMCMs were plated on laminin-coated glass coverslips in medium 199 supplemented with 5% FCS and 10 mmol/L 2,3-butanedione monoxime. After 3 hours, cells were switched to medium 199, transferred to a custom-made perfusion chamber, and electrically stimulated as described above. For each cell, a rectangular region of interest including 15-20 sarcomeres was defined and changes in sarcomere length were registered with a variable-rate CCD video camera (MyoCam-S, IonOptix) connected to an inverted microscope (Olympus IX71). The Fast Fourier Transform (FFT) algorithm was used to record changes in sarcomere length during electrically paced contractions. Data from 20-30 twitches per cell were averaged. Contraction amplitude, maximum shortening velocity, and maximum relaxation velocity were analyzed with IonWizard 6.5 software (IonOptics).

Human Plasma Samples. EDTA-treated plasma samples were obtained from 11 patients (age range of 76-86 years, 3 male and 8 female) with echocardiographic evidence of severe high-gradient aortic stenosis (valve area 0.65±0.05 cm², mean pressure gradient 51±3 mmHg) who were scheduled to undergo elective transcatheter aortic valve implantation (TAVI) at Hannover Medical School. Patients with coronary artery disease (any luminal diameter stenosis >50%), active inflammatory or malignant disease, an estimated glomerular filtration rate below 30 mL/min/1.73 m², or signs of cardiac decompensation were excluded. A second plasma sample was drawn during a routine follow-up examination 3 months after TAVI. In addition, EDTA plasma samples were obtained from 13 apparently healthy individuals (75-84 years, 3 male and 10 female) who were recruited at the University of Heidelberg (Giannitsis et al. Clin Biochem. 2020; 78:18-24). Plasma samples were stored at −80° C. All participants provided written informed consent, and the local ethics committees approved the study.

Statistical analyses. Mouse littermates were randomly allocated to the different experimental groups. Based on visual inspection, data were normally distributed with similar variances in the different groups. With small sample sizes we did not apply statistical tests for normality or equality of variances. Data are presented as mean±s.e.m. unless otherwise stated. The 2-independent-sample t test was used to compare 2 groups. For comparisons among more than 2 groups, 1-way ANOVA was used if there was 1 independent variable and 2-way ANOVA if there were 2 independent variables. Dunnett's post hoc test was used for multiple comparisons with a single control group. Tukey's post hoc test was used to adjust for multiple comparisons. A 2-tailed P value less than 0.05 was considered to indicate statistical significance. K.C.W. had full access to all data in the study and takes responsibility for the integrity of the data and the data analysis.

### Example 1

The MYDGF protein (human Factor 1; C19orf10) was identified as detailed in WO 2014/111458. The nucleic acid sequence encoding human Factor 1 is available under NCBI Gene ID: 56005 (SEQ ID NO: 6). The amino acid sequence of human Factor 1 including the N-terminal signal peptide is detailed in SEQ ID NO: 3. In the examples, human MYDGF without the signal peptide was used and expressed as detailed in Ebenhoch R. et al., Crystal structure and receptor-interacting residues of MYDGF—a protein mediating ischemic tissue repair (Nat Commun. 2019 Nov. 26; 10(1):5379 and Polten et al. Plasma Concentrations of Myeloid-Derived Growth Factor in Healthy Individuals and Patients with Acute Myocardial Infarction as Assessed by Multiple Reaction Monitoring-Mass Spectrometry. Anal Chem. 2019 Jan. 15; 91(2):1302-1308).

Mouse Homologue to Human MYDGF Used in the Examples:

Mus musculus DNA segment, Chr 17, Wayne State University 104, expressed (D17Wsu104e). The nucleic acid sequence encoding mouse Factor 1 is available under NCBI Reference Sequence: NM_080837.2 (SEQ ID NO: 7). The amino acid sequence of mouse Factor 1 including the N-terminal signal peptide is detailed in SEQ ID NO: 4. Since the N-terminal signal peptide has no relevant biological function, in the present invention, mouse Mydgf without the N-terminal peptide according to SEQ ID NO: 2 was used. To this end, the murine Mydgf cDNA sequence (containing the endogenous N-terminal signal peptide and a C-terminal 6×His-tag) was cloned into the pFlpBtM-II plasmid vector and expressed in HEK 293-6E cells (Meyer S, et al. Multi-host expression system for recombinant production of challenging proteins. PLoS One. 2013; 8:e68674). Murine Mydgf lacking the signal peptide was purified from the conditioned cell supernatant using affinity chromatography and size exclusion chromatography. For purifying recombinant Mydgf, a 6×His-tag was added to the protein.

A sequence comparison between human MYDGF (SEQ ID NO: 1) and mouse Mydgf (SEQ ID NO: 2) reveals a 92% sequence identity between both amino acid sequences.

### Example 2

Mouse and human myeloid-derived growth factor dose-dependently inhibits hypertrophy (end point, cell area) of endothelin 1 (ET1)-stimulated neonatal rat ventricular myocytes (NRVMs). NRVMs were stimulated for 24 hours with 100 nmol/L ET (purchased from Sigma-Aldrich, used here and below) and/or recombinant mouse or human myeloid-derived growth factor, respectively (Mydgf, produced in HEK 293 cells, used here and below/MYDGF; produced in BILK 293 cells as described in Ebenhoch R. et al., Crystal structure and receptor-interacting residues of MYDGF—a protein mediating ischemic tissue repair. Nat Commun. 2019 Nov. 26; 10(1):5379 and Polten F. et al. Plasma Concentrations of Myeloid-Derived Growth Factor in Healthy Individuals and Patients with Acute Myocardial Infarction as Assessed by Multiple Reaction Monitoring-Mass Spectrometry. Anal Chem. 2019 Jan. 15; 91(2):1302-1308, here and below). ET1 is a peptide hormone and prototypical inducer of cardiomyocyte hypertrophy in vitro and in vivo (reviewed in Heineke J & Molkentin D. Regulation of cardiac hypertrophy by intracellular signaling pathways. Nat Rev Mol Cell Biol 2006; 7:589-600). Cardiomyocyte hypertrophy was determined by planimetry (end point, cell area). Results are shown in table 2 below.

TABLE 2
Cell area of NRVMs cultured in the absence (control) or
presence of ET1 and/or Mydgf or MYDGF, respectively

| | Number of | Cell area | |
|---|---|---|---|
| | independent | Mean ± SEM | |
| | experiments | (% of control) | P value |
| Control | 6 | 100 ± 4 | |
| Mydgf (100 ng/mL) | 6 | 107 ± 3 | |
| ET1 | 9 | 160 ± 4 | <0.001 vs. control |
| ET1 + Mydgf (3 ng/mL) | 6 | 147 ± 3 | |
| ET1 + Mydgf (10 ng/mL) | 5 | 124 ± 3 | <0.01 vs. ET1 |
| ET1 + Mydgf (30 ng/mL) | 6 | 119 ± 4 | <0.001 vs. ET1 |
| ET1 + Mydgf (100 ng/mL) | 9 | 116 ± 1 | <0.001 vs. ET1 |
| ET1 + Mydgf (300 ng/mL) | 6 | 116 ± 2 | <0.001 vs. ET1 |
| Control | 4 | 100 ± 2 | |
| MYDGF (100 ng/mL) | 4 | 102 ± 3 | |
| ET1 | 4 | 141 ± 2 | <0.001 vs. control |
| ET1 + MYDGF (3 ng/mL) | 4 | 134 ± 1 | |
| ET1 + MYDGF (10 ng/mL) | 4 | 121 ± 1 | <0.05 vs. ET1 |
| ET1 + MYDGF (30 ng/mL) | 4 | 116 ± 1 | <0.01 vs. ET1 |
| ET1 + MYDGF (100 ng/mL) | 4 | 111 ± 2 | <0.001 vs. ET1 |
| ET1 + MYDGF (300 ng/mL) | 4 | 117 ± 2 | <0.01 vs. ET1 |

### Example 3

Mouse and human myeloid-derived growth factor inhibits hypertrophy (end point, protein content) of endothelin 1 (ET1) stimulated neonatal rat ventricular myocytes (NRVMs). NRVMs were stimulated for 24 hours with 100 nmol/L ET1 and/or 100 ng/mL recombinant mouse or human myeloid-derived growth factor, respectively (i.e. Mydgf or MYDGF, respectively). Results are shown in table 3 below.

TABLE 3
Protein content of NRVMs cultured in the absence (control)
or presence of ET1 and/or Mydgf or MYDGF, respectively

| | Number of | Protein content | |
|---|---|---|---|
| | independent | Mean ± SEM | |
| | experiments | (% of control) | P value |
| Control | 3 | 100 ± 5 | |
| Mydgf | 3 | 78 ± 8 | |
| ET1 | 3 | 165 ± 5 | <0.001 vs. control |
| ET1 + Mydgf | 3 | 104 ± 2 | <0.01 vs. ET1 |
| Control | 6 | 100 ± 7 | |
| MYDGF | 6 | 111 ± 7 | |
| ET1 | 6 | 194 ± 13 | <0.001 vs. control |
| ET1 + MYDGF | 6 | 136 ± 7 | <0.01 vs. ET1 |

### Example 4

Mouse myeloid-derived growth factor inhibits hypertrophy (end point, cell size) of endothelin 1 (ET1) stimulated adult rat ventricular myocytes (ARVMs). ARVMs were stimulated for 24 hours with 100 nmol/L ET1 and/or 100 ng/mL recombinant mouse myeloid-derived growth factor (i.e. Mydgf). Cardiomyocyte hypertrophy was determined by morphometry (cell length and cell width) and planimetry (cell area). Results are shown in table 4 below.

TABLE 4
Cell size of ARVMs cultured in the absence
(control) or presence of ET1 and/or Mydgf

| | | |
|---|---|---|
| Number of | | |
| independent | | |
| experiments | Mean ± SEM | P value |

Cell area (μm²)

| | | | |
|---|---|---|---|
| Control | 3 | 1838 ± 55 | |
| Mydgf | 3 | 1711 ± 71 | |
| ET1 | 3 | 2285 ± 64 | <0.01 vs. control |
| ET1 + Mydgf | 3 | 1821 ± 39 | <0.01 vs. ET1 |

Cell length (μm)

| | | |
|---|---|---|
| Control | 3 | 120 ± 2 |
| Mydgf | 3 | 124 ± 2 |
| ET1 | 3 | 124 ± 2 |
| ET1 + Mydgf | 3 | 120 ± 1 |

Cell width (μm)

| | | | |
|---|---|---|---|
| Control | 3 | 18 ± 1 | |
| Mydgf | 3 | 15 ± 1 | |
| ET1 | 3 | 23 ± 1 | <0.05 vs. control |
| ET1 + Mydgf | 3 | 17 ± 1 | <0.05 vs. ET1 |

### Example 5

Mouse myeloid-derived growth factor increases sarco/endoplasmic reticulum Ca²⁺-ATPase (Serca2a) protein expression in endothelin 1 (ET1)-stimulated neonatal rat ventricular myocytes (NRVMs). NRVMs were stimulated for 24 hours with 100 nmol/L ET1 and/or 100 ng/mL recombinant mouse myeloid-derived growth factor (i.e. Mydgf). Serca2a and vinculin protein expression levels were then determined by immunoblotting. Serca2a is a critical regulator of calcium homeostasis in cardiomyocytes. In experimental heart failure models and in human heart failure, Serca2a (SERCA2a in humans) expression in cardiomyocytes is decreased, thus promoting functional decline and heart failure. SERCA2a has therefore been proposed as a treatment target in heart failure (reviewed in Kawase Y & Hajjar R J. The cardiac sarcoplasmic/endoplasmic reticulum calcium ATPase: a potent target for cardiovascular diseases. Nat Clin Pract Cardiovasc Med 2008; 5:554-65). Results are shown in table 5 below.

TABLE 5
Serca2a protein expression (normalized to vinculin
protein expression) in NRVMs cultured in the absence
(control) or presence of ET1 and/or Mydgf

| | | Serca2a protein | |
|---|---|---|---|
| | Number of | expression | |
| | independent | Mean ± SEM | |
| | experiments | (% of control) | P value |
| Control | 6 | 100 ± 6 | |
| Mydgf | 6 | 137 ± 10 | <0.05 vs. control |
| ET1 | 6 | 96 ± 6 | |
| ET1 + Mydgf | 6 | 131 ± 8 | <0.05 vs. ET1 |

### Example 6

Downregulation of sarco/endoplasmic reticulum Ca²⁺-ATPase (Serca2a) abolishes the anti-hypertrophic effects of mouse myeloid-derived growth factor in endothelin 1 (ET1)-stimulated neonatal rat ventricular myocytes (NRVMs). NRVMs were transfected with small interfering (si)RNA targeting Serca2a (purchased from Thermo Fisher Scientific, cat. no. 4390771, ID: s132037) or control siRNA (Thermo Fisher Scientific, cat. no. 4390843). Thereafter, NRVMs were stimulated for 24 hours with 100 nmol/L ET1 and/or 100 ng/mL recombinant mouse myeloid-derived growth factor (i.e.). Cardiomyocyte hypertrophy was determined by planimetry (end point, cell area). Results are shown in table 6 below.

TABLE 6
Cell size of NRVMs cultured in the absence (control)
or presence of ET1 and/or Mydgf after transfection
with Serca2a siRNA or control siRNA

| | | |
|---|---|---|
| Number of | Cell area | |
| independent | Mean ± SEM | |
| experiments | (μm²) | P value |

Control siRNA pre-treatment

| | | | |
|---|---|---|---|
| Control | 3 | 857 ± 10 | |
| (no stimulation) | | | |
| Mydgf | 3 | 799 ± 90 | |
| ET1 | 3 | 1371 ± 69 | <0.01 vs. |
| | | | no stimulation |
| ET1 + Mydgf | 3 | 944 ± 63 | <0.01 vs. ET1 |

Serca2a siRNA pre-treatment

| | | | |
|---|---|---|---|
| Control | 3 | 951 ± 37 | |
| (no stimulation) | | | |
| Mydgf | 3 | 980 ± 47 | |
| ET1 | 3 | 1307 ± 36 | <0.001 vs. |
| | | | no stimulation |
| ET1 + Mydgf | 3 | 1265 ± 37 | Not significant |
| | | | vs. ET1 |

### Example 7

Mouse myeloid-derived growth factor promotes anti-fibrotic effects (end points, collagen 1A1 and connective tissue growth factor [Tgfβ1] mRNA expression) in transforming growth factor β1 (Tgfβ1)-stimulated fibroblasts isolated from neonatal rat ventricles (NRVFs). NRVFs were stimulated for 24 hours with recombinant mouse Tgfβ1 (2 ng/mL; purchased from R&D Systems, used here and below) and/or 100 ng/mL recombinant mouse myeloid-derived growth factor (Mydgf). TGFβ1 is a critical inducer of organ fibrosis (reviewed in Border W A & Noble N A. Transforming growth factor beta in tissue fibrosis. N Engl J Med 1994; 331:1286-92). Results are shown in table 7 below.

TABLE 7
Collagen 1A1 und Ctgf mRNA expression (RT-
qPCR) in NRVFs cultured in the absence (control)
or presence of Tgfβ1 and/or Mydgf

| | | |
|---|---|---|
| Number of | | |
| independent | | |
| experiments | Mean ± SEM | P value |

Collagen 1A1 expression (% of control)

| | | | |
|---|---|---|---|
| Control | 3 | 100 ± 17 | |
| Mydgf | 3 | 125 ± 34 | |
| Tgfβ1 | 3 | 252 ± 16 | <0.01 vs. control |
| Tgfβ1 + Mydgf | 3 | 148 ± 18 | <0.05 vs. Tgfβ1 |

CTGF expression (% of control)

| | | | |
|---|---|---|---|
| Control | 3 | 100 ± 28 | |
| Mydgf | 3 | 147 ± 38 | |
| Tgfβ1 | 3 | 235 ± 18 | <0.05 vs. control |
| Tgfβ1 + Mydgf | 3 | 81 ± 39 | <0.05 vs. Tgfβ1 |

### Example 8

Mouse myeloid-derived growth factor promotes anti-fibrotic effects (end point, α-smooth muscle actin [SMA] promoter activity) in transforming growth factor β1 (Tgfβ1)-stimulated fibroblasts isolated from neonatal rat ventricles (NRVF). NRVFs were transfected with a reporter plasmid encoding firefly luciferase under the control of a human αSMA promoter fragment (−259/+51 base pairs) and were then stimulated for 24 hours with recombinant mouse Tgfβ1 (2 ng/mL) and/or 100 ng/mL recombinant mouse myeloid-derived growth factor (Mydgf). Results are shown in table 8 below.

TABLE 8
αSMA promoter activity in NRVFs cultured in the absence
(control) or presence of Tgfβ1 and/or Mydgf

| | | αSMA promoter | |
|---|---|---|---|
| | Number of | activity | |
| | independent | Mean ± SEM | |
| | experiments | (% of control) | P value |
| Control | 8 | 100 ± 19 | |
| Mydgf | 8 | 133 ± 18 | |
| Tgfβ1 | 8 | 712 ± 32 | <0.001 vs. control |
| Tgfβ1 + Mydgf | 8 | 467 ± 42 | <0.001 vs. Tgfβ1 |

### Example 9

Human myeloid-derived growth factor partially attenuates transforming growth factor β1 (Tgf β1)-induced gene expression changes in fibroblasts isolated from neonatal rat ventricles (NRVF). NRVFs were co-incubated with recombinant mouse Tgfβ 1 (2 ng/mL) and/or 100 ng/mL recombinant human myeloid-derived growth factor (MYDGF) for 4 hours. PolyA RNA was isolated, converted into cDNA, and underwent library preparation before gene expression was profiled using next generation sequencing. The reads were aligned to the rat reference genome, the number of reads mapped to each gene was counted, and differential gene (indicated in the table 9) across the different treatment conditions were computed with the Bioconductor limma package, applying the corrected p-value cutoff of 0.1, and fold change cutoff of 1.5 . . . 26 Tgfβ 1 downregulated genes were upregulated by MYDGF and 30 Tgfβ 1 induced genes were downregulated by MYDGF.

TABLE 9

| | | | TGFb 1 vs. Control | | | MYDGF + TGFb 1 vs. TGF b 1 | | |
|---|---|---|---|---|---|---|---|---|
| | | | log2 fold | | adjusted | log2 fold | | adjusted |
| Ensembl no. | Entrez no. | Symbol | change | p value | p value | change | p value | p value |
| ENSRNOG00000010047 | 140582 | Ddit4l | −2.812761867 | 3.77E−12 | 1.45E−10 | 1.52600617 | 0.000426049 | 0.021683208 |
| ENSRNOG00000025624 | 367085 | Arhgap20 | −2.923396234 | 3.37E−38 | 7.86E−36 | 1.435616525 | 1.40E−09 | 4.21E−07 |
| ENSRNOG00000029768 | 287562 | Ccl12 | −2.083108672 | 2.29E−08 | 5.46E−07 | 1.246021285 | 0.001052384 | 0.046452616 |
| ENSRNOG00000037082 | 310782 | Mybphl | −1.609166645 | 1.44E−15 | 7.69E−14 | 1.129579176 | 5.65E−08 | 1.15E−05 |
| ENSRNOG00000002771 | 59325 | Ereg | −1.833195893 | 7.96E−11 | 2.65E−09 | 1.076789668 | 0.000232694 | 0.013273145 |
| ENSRNOG00000010308 | 113984 | Nr2f2 | −1.442120196 | 6.38E−46 | 2.18E−43 | 1.000033277 | 1.48E−22 | 3.11E−19 |
| ENSRNOG00000006569 | 362800 | Itgb8 | −1.032689837 | 1.25E−05 | 0.000186492 | 0.975203941 | 3.66E−05 | 0.00291766 |
| ENSRNOG00000057794 | 304135 | Adamts5 | −1.762562535 | 3.54E−25 | 4.39E−23 | 0.898314638 | 1.57E−07 | 2.70E−05 |
| ENSRNOG00000032396 | 287231 | RGD1559575 | −0.792009963 | 0.007679422 | 0.049138468 | 0.867724628 | 0.003041748 | 0.098388569 |
| ENSRNOG00000004033 | 361324 | Sema6a | −1.823280646 | 4.07E−19 | 3.09E−17 | 0.812021871 | 9.84E−05 | 0.00654701 |
| ENSRNOG00000020904 | 309175 | Cdc42ep2 | −1.473108316 | 3.94E−34 | 8.24E−32 | 0.751091175 | 1.77E−09 | 5.09E−07 |
| ENSRNOG00000008336 | 25341 | Tnfrsf11b | −1.644928606 | 5.67E−191 | 3.97E−187 | 0.747009664 | 3.18E−40 | 2.00E−36 |
| ENSRNOG00000002381 | 25667 | Bmp3 | −1.583132157 | 8.05E−32 | 1.54E−29 | 0.745213673 | 6.76E−08 | 1.31E−05 |
| ENSRNOG00000006019 | 289388 | G0s2 | −1.674729992 | 1.05E−13 | 4.74E−12 | 0.741857524 | 0.001659897 | 0.064671706 |
| ENSRNOG00000009482 | 499380 | Emx2 | −1.08642493 | 6.47E−08 | 1.44E−06 | 0.721127386 | 0.000427459 | 0.021683208 |
| ENSRNOG00000011136 | 315039 | Osr2 | −0.735320588 | 0.000634476 | 0.006077244 | 0.700279447 | 0.001100292 | 0.048389741 |
| ENSRNOG00000013306 | 306081 | Pcdh20 | −0.817305837 | 1.59E−12 | 6.29E−11 | 0.693908597 | 2.15E−09 | 5.75E−07 |
| ENSRNOG00000047699 | 25554 | Snai2 | −0.736414335 | 1.05E−23 | 1.13E−21 | 0.682561737 | 1.17E−20 | 1.84E−17 |
| ENSRNOG00000004317 | 29555 | Vipr2 | −1.297086252 | 5.09E−29 | 8.58E−27 | 0.680780156 | 1.41E−08 | 3.23E−06 |
| ENSRNOG00000000906 | 360757 | Medag | −2.035407803 | 1.60E−48 | 6.59E−46 | 0.672788094 | 7.25E−06 | 0.000765992 |
| ENSRNOG00000003929 | 317183 | Pcdh19 | −1.348884511 | 1.84E−21 | 1.76E−19 | 0.637589323 | 1.09E−05 | 0.001066687 |
| ENSRNOG00000008785 | 84410 | Klf5 | −0.827267237 | 2.27E−07 | 4.62E−06 | 0.635712124 | 8.32E−05 | 0.005750261 |
| ENSRNOG00000020546 | 25330 | Lipe | −1.114731561 | 2.48E−10 | 7.81E−09 | 0.635532422 | 0.000517103 | 0.02561083 |
| ENSRNOG00000027030 | 25026 | Adm | −1.155301212 | 9.62E−37 | 2.10E−34 | 0.634092879 | 7.06E−12 | 3.29E−09 |
| ENSRNOG00000026607 | NA | Tnfsf18 | −1.818921178 | 6.28E−59 | 3.99E−56 | 0.62533476 | 3.25E−08 | 6.94E−06 |
| ENSRNOG00000014874 | 305454 | Zfyve28 | −1.694362265 | 3.00E−28 | 4.48E−26 | 0.619012229 | 0.000113017 | 0.0073666 |
| ENSRNOG00000018858 | 292264 | Myct1 | 1.630038156 | 3.74E−28 | 5.50E−26 | −0.615641506 | 7.68E−07 | 0.000109569 |
| ENSRNOG00000060773 | 360899 | Sertad4 | 1.061766796 | 2.95E−46 | 1.03E−43 | −0.64373987 | 2.09E−18 | 2.92E−15 |
| ENSRNOG00000058329 | 113931 | Prrx2 | 0.739261343 | 1.08E−06 | 1.97E−05 | −0.665650907 | 8.52E−06 | 0.000878751 |
| ENSRNOG00000052486 | 64358 | Kcna6 | 1.554760933 | 1.26E−33 | 2.51E−31 | −0.670163862 | 1.98E−08 | 4.37E−06 |
| ENSRNOG00000015441 | 25084 | Il4r | 1.102247467 | 1.31E−65 | 1.23E−62 | −0.672879612 | 6.88E−27 | 1.73E−23 |
| ENSRNOG00000019390 | 316088 | Klhl40 | 2.451156465 | 3.84E−25 | 4.72E−23 | −0.678477305 | 2.47E−05 | 0.002044872 |
| ENSRNOG00000001979 | 266766 | Rcan1 | 1.253706503 | 5.05E−27 | 7.00E−25 | −0.67924198 | 3.18E−09 | 8.34E−07 |
| ENSRNOG00000042838 | 24517 | Junb | 0.706774333 | 1.59E−13 | 7.01E−12 | −0.693007988 | 4.15E−13 | 2.27E−10 |
| ENSRNOG00000000827 | 294235 | Ier3 | 0.976232517 | 3.11E−15 | 1.62E−13 | −0.699701738 | 1.37E−08 | 3.19E−06 |
| ENSRNOG00000053113 | NA | Rn50_1_0435.2 | 1.775351734 | 1.36E−15 | 7.34E−14 | −0.716243601 | 0.000328525 | 0.01729223 |
| ENSRNOG00000000156 | 65049 | LOC100911486 | 2.121240906 | 5.64E−19 | 4.18E−17 | −0.720265885 | 0.000355969 | 0.018658692 |
| ENSRNOG00000013683 | 29733 | S1pr1 | 1.182602471 | 3.05E−16 | 1.72E−14 | −0.728559362 | 4.41E−08 | 9.24E−06 |
| ENSRNOG00000014197 | 500578 | Tmem51 | 1.018751726 | 2.04E−15 | 1.08E−13 | −0.730126734 | 6.76E−09 | 1.64E−06 |
| ENSRNOG00000002227 | 64030 | Kit | 1.77921558 | 2.51E−27 | 3.51E−25 | −0.737695719 | 1.55E−07 | 2.70E−05 |
| ENSRNOG00000013166 | 84426 | Wnt4 | 1.914205655 | 8.00E−52 | 4.00E−49 | −0.740049809 | 1.11E−09 | 3.48E−07 |
| ENSRNOG00000002072 | 360695 | Eva1c | 0.831960971 | 6.06E−05 | 0.000778634 | −0.745330988 | 0.000238057 | 0.013369425 |
| ENSRNOG00000007159 | 24770 | Ccl2 | 1.243501309 | 1.96E−16 | 1.13E−14 | −0.756950717 | 4.72E−07 | 6.99E−05 |
| ENSRNOG00000000034 | 289419 | Nuak2 | 1.069346085 | 1.56E−21 | 1.51E−19 | −0.786139167 | 7.92E−13 | 4.15E−10 |
| ENSRNOG00000007118 | 500221 | Eva1a | 1.414031637 | 6.16E−12 | 2.32E−10 | −0.802408121 | 2.72E−05 | 0.002220679 |
| ENSRNOG00000007637 | 313339 | Acer2 | 1.001219486 | 1.46E−07 | 3.05E−06 | −0.80911866 | 1.00E−05 | 0.001006667 |
| ENSRNOG00000008323 | 287467 | Pitpnm3 | 1.58842341 | 5.87E−14 | 2.73E−12 | −0.813876235 | 1.64E−05 | 0.001477074 |
| ENSRNOG00000018003 | 116677 | F2rl1 | 1.089740553 | 0.000129571 | 0.001531432 | −0.842431516 | 0.001785917 | 0.068081333 |
| ENSRNOG00000007002 | 60584 | Lif | 1.631933844 | 2.85E−40 | 7.26E−38 | −0.857505491 | 3.48E−15 | 2.92E−12 |
| ENSRNOG00000000640 | 114090 | Egr2 | 1.915116308 | 7.82E−28 | 1.12E−25 | −0.8827439 | 1.46E−08 | 3.29E−06 |
| ENSRNOG00000056219 | 140914 | Olr1 | 2.35531464 | 4.69E−234 | 6.56E−230 | −0.885976176 | 1.30E−35 | 5.44E−32 |
| ENSRNOG00000013515 | 116680 | Ptpru | 1.413511763 | 1.12E−06 | 2.04E−05 | −0.905951754 | 0.00096919 | 0.043700407 |
| ENSRNOG00000005332 | 266600 | Csdc2 | 1.468181877 | 9.18E−12 | 3.43E−10 | −0.915185463 | 4.57E−06 | 0.00050899 |
| ENSRNOG00000007687 | 315711 | Sema7a | 1.757693473 | 5.81E−29 | 9.56E−27 | −0.944080953 | 2.40E−11 | 1.04E−08 |
| ENSRNOG00000043098 | 689415 | Mt2A | 1.131345161 | 0.000110791 | 0.001335405 | −1.339742471 | 5.34E−06 | 0.000573766 |
| ENSRNOG00000038047 | 24567 | Mt1 | 1.771916419 | 8.31E−06 | 0.000128422 | −1.95106387 | 1.13E−06 | 0.000150773 |

### Example 10

Mouse myeloid-derived growth factor protein therapy promotes anti-hypertrophic and anti-fibrotic effects in mice subjected to transverse aortic constriction (TAC). C57BL6/N wild type mice underwent TAC surgery (first described in Rockman H A et al. Segregation of atrial-specific and inducible expression of an atrial natriuretic factor transgene in an in vivo mouse model of cardiac hypertrophy. Proc Natl Acad Sci USA 1991; 88:8277-81). TAC exposes the left ventricle of the heart to chronic pressure overload and results in left ventricular (LV) hypertrophy and interstitial fibrosis (reviewed in Houser S R et al. Animal models of heart failure: A scientific statement from the American Heart Association. Circ Res 2012; 111:131-50). Control mice underwent sham surgery (thoracotomy without aortic constriction). Immediately after TAC, mice received a 10 tag, intraperitoneal bolus injection of recombinant mouse myeloid-derived growth factor (Mydgf). Thereafter, mice were treated with a 7 day subcutaneous infusion of Mydgf (10 μg/day via osmotic mini pumps). Additional TAC mice were injected and infused with vehicle only (0.9% NaCl). After 42 days, LV hypertrophy was determined by gravimetry (endpoint, LV mass/body mass using a commercially available standard balance); LV interstitial fibrosis was quantified by Sirius red staining. Results are shown in table 10 below.

TABLE 10
LV hypertrophy and interstitial fibrosis after sham surgery or TAC

| | | |
|---|---|---|
| Number | | |
| of mice | Mean ± SEM | P value |

LV mass/body mass (mg/g)

| | | | |
|---|---|---|---|
| Sham surgery | 5 | 4.9 ± 0.3 | |
| TAC, NaCl | 7 | 9.0 ± 0.7 | <0.001 vs. sham |
| TAC, Mydgf | 7 | 6.9 ± 0.3 | <0.05 vs. TAC, NaCl |

LV interstitial fibrosis (% of sham)

| | | | |
|---|---|---|---|
| Sham surgery | 5 | 100 ± 2 | |
| TAC, NaCl | 7 | 315 ± 9 | <0.001 vs. sham |
| TAC, Mydgf | 7 | 183 ± 9 | <0.001 vs. TAC, NaCl |

### Example 11

Mouse myeloid-derived growth factor protein therapy improves heart function in mice subjected to transverse aortic constriction (TAC). C57BL6/N wild type mice underwent TAC surgery. Control mice underwent sham surgery (thoracotomy without aortic constriction). Immediately after TAC, mice received a 10 μg, intraperitoneal bolus injection of recombinant mouse myeloid-derived growth factor (Mydgf). Thereafter, mice were treated with a 7 day subcutaneous infusion of Mydgf (10 μg/day via osmotic mini pumps). Additional TAC mice were injected and infused with vehicle only (0.9% NaCl). After 42 days, mice underwent high-resolution transthoracic 2D echocardiography using a linear 30 MHz transducer (Vevo 3100, VisualSonics). Left ventricular (LV) end-diastolic area (LVEDA) and left ventricular end-systolic area (LVESA) were determined from a long axis view. Fractional area change (FAC) was calculated as a measure of systolic function [(LVEDA−LVESA)/LVEDA]×100. Results are shown in table 11 below.

TABLE 11
LV dimensions and systolic function after sham surgery or TAC

| | Number | | |
|---|---|---|---|
| | of mice | Mean ± SEM | P value |
| LVEDA (mm²) | | | |
| Sham surgery | 10 | 23.2 ± 0.4 | |
| TAC, NaCl | 6 | 34.2 ± 2.3 | <0.01 vs. sham |
| TAC, Mydgf | 9 | 29.2 ± 1.5 | |
| LVESA (mm²) | | | |
| Sham surgery | 10 | 12.1 ± 0.2 | |
| TAC, NaCl | 6 | 32.1 ± 2.5 | <0.001 vs. sham |
| TAC, Mydgf | 9 | 23.1 ± 1.8 | <0.05 vs. TAC, NaCl |
| FAC (%) | | | |
| Sham surgery | 10 | 48 ± 1 | |
| TAC, NaCl | 6 | 6 ± 1 | <0.001 vs. sham |
| TAC, Mydgf | 9 | 18 ± 3 | <0.01 vs. TAC, NaCl |

### Example 12

Mouse myeloid-derived growth factor protein therapy increases sarco/endoplasmic reticulum Ca²⁺-ATPase (Serca2a) protein expression in left ventricular cardiomyocytes isolated from mice subjected to transverse aortic constriction (TAC). C57BL6/N wild type mice underwent TAC surgery. Control mice underwent sham surgery (thoracotomy without aortic constriction). Immediately after TAC, mice received a 10 μg, intraperitoneal bolus injection of recombinant mouse myeloid-derived growth factor (Mydgf). Thereafter, mice were treated with a 7 day subcutaneous infusion of Mydgf (10 μg/day via osmotic mini pumps). Additional TAC mice were injected and infused with vehicle only (0.9% NaCl). After 7 days, Serca2a and β-actin protein expression levels were measured by immunoblotting in isolated left ventricular cardiomyocytes. Results are shown in table 12 below.

TABLE 12
Serca2a protein expression (normalized to β-actin protein expression)
in left ventricular cardiomyocytes after sham surgery or TAC

| | | Serca2a expression | |
|---|---|---|---|
| | Number | Mean ± SEM | |
| | of mice | (% of control) | P value |
| Sham surgery | 4 | 100 ± 9 | |
| TAC, NaCl | 5 | 61 ± 13 | <0.05 vs. sham |
| TAC, Mydgf | 5 | 107 ± 15 | <0.05 vs. TAC, NaCl |

### Example 13

Mouse myeloid-derived growth factor protein therapy reduces left ventricular cardiomyocyte hypertrophy in mice subjected to transverse aortic constriction (TAC). C57BL6/N wild type mice underwent TAC surgery. Control mice underwent sham surgery (thoracotomy without aortic constriction). Immediately after TAC, mice received a 10 μg, intraperitoneal bolus injection of recombinant mouse myeloid-derived growth factor (Mydgf). Thereafter, mice were treated with a 7 day subcutaneous infusion of Mydgf (10 μg/day via osmotic mini pumps). Additional TAC mice were injected and infused with vehicle only (0.9% NaCl). After 42 days, left ventricular cardiomyocyte cross-sectional area was determined in wheat germ agglutinin-stained tissue sections. Results are shown in table 13 below.

TABLE 13
Cardiomyocyte cross-sectional area after TAC

| | Number | Mean ± SEM | |
|---|---|---|---|
| | of mice | (μm²) | P value |
| TAC, NaCl | 6 | 653 ± 27 | |
| TAC, Mydgf | 6 | 527 ± 26 | <0.01 vs. TAC, NaCl |

### Example 14

Mouse myeloid-derived growth factor gene therapy improves heart function and promotes anti-hypertrophic and anti-fibrotic effects in mice subjected to transverse aortic constriction (TAC). C57BL6/N wild type mice were lethally irradiated and transplanted with bone marrow stem cells that had been transduced with a lentivirus encoding mouse myeloid-derived growth factor (Mydgf) or a lentivirus encoding green fluorescent protein (GFP control). This lentiviral system has previously been described (Magnusson M et al. HOXA10 is a critical regulator for hematopoietic stem cells and erythroid/megakaryocyte development. Blood 2007; 109:3687-96). 6 weeks after transplantation, mice underwent TAC surgery. Control mice underwent sham surgery (thoracotomy without aortic constriction). After 42 days, mice underwent high-resolution transthoracic 2D echocardiography using a linear 30 MHz transducer (Vevo 3100, VisualSonics). Left ventricular (LV) end-diastolic area (LVEDA) and left ventricular end-systolic area (LVESA) were determined from a long axis view. Fractional area change (FAC) was calculated as a measure of systolic function [(LVEDA−LVESA)/LVEDA]×100. Left ventricular end-diastolic posterior wall thickness as a measure of left ventricular hypertrophy was determined from a short axis view. LV interstitial fibrosis was quantified by Sirius red staining. Results are shown in table 14 below.

TABLE 14
LV systolic function, hypertrophy, and interstitial
fibrosis after sham surgery or TAC

| | | |
|---|---|---|
| Number of | | |
| mice | Mean ± SEM | P value |

FAC (%)

| | | | |
|---|---|---|---|
| Sham surgery | 10 | 53 ± 2 | |
| TAC, GFP-control | 6 | 13 ± 3 | <0.001 vs. sham |
| lentivirus | | | |
| TAC, Mydgf | 6 | 33 ± 5 | <0.01 vs. TAC, |
| lentivirus | | | GFP-control |

Left ventricular wall thickness (mm)

| | | | |
|---|---|---|---|
| Sham surgery | 10 | 0.5 ± 0.02 | |
| TAC, GFP-control | 6 | 1.0 ± 0.03 | <0.001 vs. sham |
| lentivirus | | | |
| TAC, Mydgf | 6 | 0.8 ± 0.04 | <0.001 vs. TAC, |
| lentivirus | | | GFP-control |

LV interstitial fibrosis (% of sham)

| | | | |
|---|---|---|---|
| Sham surgery | 3 | 100 ± 47 | |
| TAC, GFP-control | 13 | 976 ± 159 | <0.05 vs. sham |
| lentivirus | | | |
| TAC, Mydgf | 15 | 510 ± 148 | <0.05 vs. TAC, |
| lentivirus | | | GFP-control |

### Example 15

Human myeloid-derived growth factor inhibits transforming growth factor β1 (TGFβ1)-stimulated SMAD phosphorylation in lung fibroblasts from patients with idiopathic pulmonary fibrosis. Lung fibroblasts from two patients with idiopathic pulmonary fibrosis were stimulated for 15, 30, or 60 min with 2 ng/mL recombinant human TGFβ1 (purchased from R&D Systems, used here and below) in the absence or presence of 100 ng/mL recombinant human myeloid-derived growth factor (MYDGF; produced in 13K 293 cells, used here and below). SMAD phosphorylation (activation) was determined by immunoblotting. The SMAD signaling pathway is a critical mediator of TGFβ1's pro fibrotic effects (reviewed in Walton K L et al. Targeting TGFβ mediated SMAD signaling for the prevention of fibrosis. Front Pharmacol 2017; 8:461). Results are shown in table 15 below.

TABLE 15
SMAD2 (Ser465/467) and SMAD3 (Ser423/425) phosphorylation
(normalized to the expression of α-tubulin) in
lung fibroblasts from two patients with idiopathic
pulmonary fibrosis cultured in the absence (control)
or presence of TGFβ1 and/or MYDGF

| | |
|---|---|
| Phospho-SMAD2 | Phospho-SMAD3 |
| (% of control) | (% of control) |

SMAD2/3 phosphorylation (% of control), patient #1

| | | |
|---|---|---|
| Control | 100 | 100 |
| MYDGF | 166 | 113 |
| TGFβ1 (15 min) | 717 | 1849 |
| TGFβ1 (30 min) | 1128 | 1788 |
| TGFβ1 (60 min) | 1223 | 1564 |
| TGFβ1 + MYDGF (15 min) | 592 | 895 |
| TGFβ1 + MYDGF (30 min) | 991 | 1509 |
| TGFβ1 + MYDGF (60 min) | 609 | 1116 |

SMAD2/3 phosphorylation (% of control), patient #2

| | | |
|---|---|---|
| Control | 100 | 100 |
| MYDGF | 79 | 88 |
| TGFβ1 (15 min) | 2094 | 979 |
| TGFβ1 (30 min) | 2201 | 1302 |
| TGFβ1 (60 min) | 2044 | 724 |
| TGFβ1 + MYDGF (15 min) | 309 | 919 |
| TGFβ1 + MYDGF (30 min) | 354 | 807 |
| TGFβ1 + MYDGF (60 min) | 35 | 496 |

### Example 16

Human myeloid-derived growth factor inhibits migration of lung fibroblasts from patients with idiopathic pulmonary fibrosis. Lung fibroblasts from two patients with idiopathic pulmonary fibrosis were grown to confluency. Monolayers were scratched with a 200 μL pipet tip, washed, and cultured for 16 hours in the absence or presence of 2 ng/mL human transforming growth factor β1 (TGFβ1) and/or 100 ng/mL recombinant human myeloid-derived growth factor (MYDGF). Before (0 hours) and after stimulation (16 hours), digital phase contrast images were captured. Recovery (%) was calculated as [(cell free area at 0 hours−cell free area at 16 hours)/cell free area at 0 hours]×100. Results are shown in table 16 below.

TABLE 16
Migration after scratch injury of lung fibroblasts from two
patients with idiopathic pulmonary fibrosis cultured in
the absence (control) or presence of TGFβ1 and/or MYDGF

| | | |
|---|---|---|
| Number of | | |
| independent | Recovery | |
| experiments | Mean ± SEM (%) | P value |

Lung fibroblasts from patient #1

| | | | |
|---|---|---|---|
| Control | 4 | 37.4 ± 1.0 | |
| MYDGF | 4 | 26.7 ± 2.6 | <0.05 vs. control |
| TGFβ1 | 4 | 38.6 ± 1.5 | |
| TGFβ1 + | 4 | 32.5 ± 2.2 | P = 0.10 vs. TGFβ1 |
| MYDGF | | | |

Lung fibroblasts from patient #2

| | | | |
|---|---|---|---|
| Control | 8 | 63.4 ± 2.2 | |
| MYDGF | 8 | 54.0 ± 2.0 | <0.05 vs. control |
| TGFβ1 | 8 | 64.2 ± 1.2 | |
| TGFβ1 + | 8 | 54.6 ± 1.3 | <0.001 vs. TGFβ1 |
| MYDGF | | | |

### Example 17

Mouse myeloid-derived growth factor inhibits migration of lung fibroblasts from patients with idiopathic pulmonary fibrosis. Lung fibroblasts from two patients with idiopathic pulmonary fibrosis were grown to confluency. Monolayers were scratched with a 200 μL pipet tip, washed, and cultured for 16 hours in the absence or presence of 2 ng/mL human transforming growth factor β1 (TGFβ1) and/or 100 ng/mL recombinant mouse myeloid-derived growth factor (Mydgf). Before (0 hours) and after stimulation (16 hours), digital phase contrast images were captured. Recovery (%) was calculated as [(cell free area at 0 hours−cell free area at 16 hours)/cell free area at 0 hours]×100. Results are shown in table 17 below.

TABLE 17
Migration after scratch injury of lung fibroblasts from two
patients with idiopathic pulmonary fibrosis cultured in
the absence (control) or presence of TGFβ1 and/or Mydgf

| | | |
|---|---|---|
| Number of | | |
| independent | Recovery | |
| experiments | Mean ± SEM (%) | P value |

Lung fibroblasts from patient #1

| | | | |
|---|---|---|---|
| Control | 3 | 47.1 ± 1.6 | |
| Mydgf | 3 | 36.3 ± 1.1 | <0.05 vs. control |
| TGFβ1 | 3 | 44.6 ± 2.7 | |
| TGFβ1 + Mydgf | 3 | 29.8 ± 2.0 | <0.05 vs. TGFβ1 |

Lung fibroblasts from patient #2

| | | | |
|---|---|---|---|
| Control | 3 | 58.6 ± 1.1 | |
| Mydgf | 3 | 44.6 ± 1.6 | <0.01 vs. control |
| TGFβ1 | 3 | 59.0 ± 3.1 | |
| TGFβ1 + Mydgf | 3 | 43.8 ± 1.5 | <0.05 vs. TGFβ1 |

### Example 18

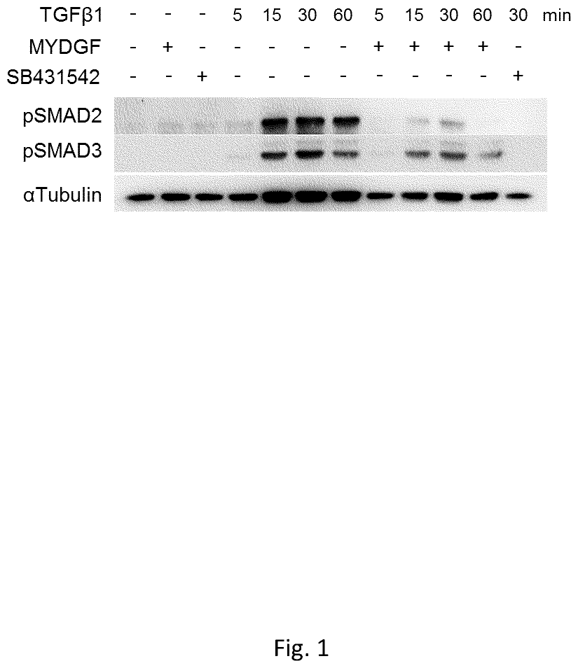

Human myeloid-derived growth factor inhibits transforming growth factor β1 (TGFβ1)-stimulated SMAD phosphorylation in lung fibroblasts from a patient with idiopathic pulmonary fibrosis. Lung fibroblasts from a patient with idiopathic pulmonary fibrosis were stimulated for 5, 15, 30, or 60 min with 2 ng/mL recombinant human TGFβ1 in the absence or presence of 100 ng/mL recombinant human myeloid-derived growth factor (MYDGF). SMAD phosphorylation (activation) was determined by immunoblotting. The small molecule ALK5/TGFβ type I receptor inhibitor SB431542 (10 μmol/L; purchased from Sigma-Aldrich, used here and below) was used as positive control. Results are shown in FIG. 1.

### Example 19

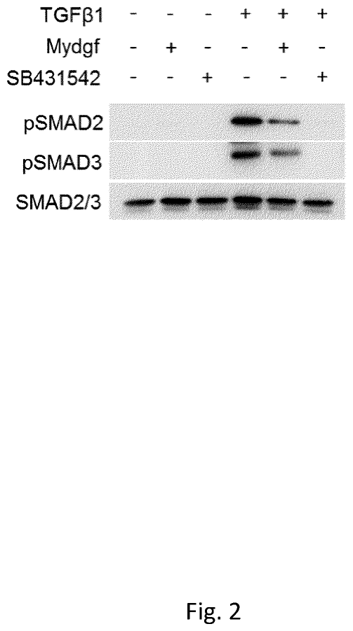

Mouse myeloid-derived growth factor inhibits transforming growth factor β1 (TGFβ1)-stimulated SMAD phosphorylation in left ventricular fibroblasts from a patient with terminal heart failure. Left ventricular fibroblasts from a patient with terminal heart failure were stimulated for 30 min with 2 ng/mL recombinant human TGFβ1 in the absence or presence of 100 ng/mL recombinant mouse myeloid-derived growth factor (Mydgf). SMAD phosphorylation (activation) was determined by immunoblotting. The small molecule ALK5/TGFβ type I receptor inhibitor SB431542 (10 μmol/L) was used as positive control. Results are shown in FIG. 2.

### Example 20

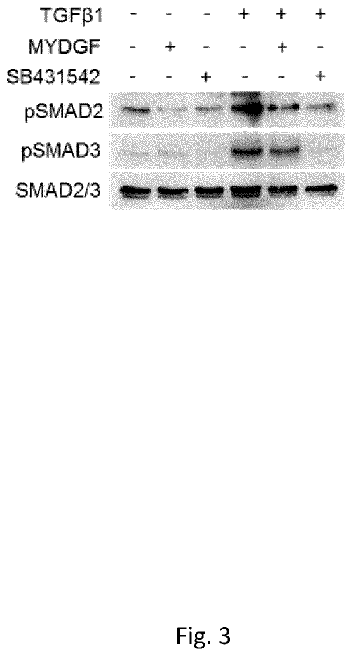

Human myeloid-derived growth factor inhibits transforming growth factor β1 (TGFβ1)-stimulated SMAD phosphorylation in left ventricular fibroblasts from a patient with terminal heart failure. Left ventricular fibroblasts from a patient with terminal heart failure were stimulated for 30 min with 2 ng/mL recombinant human TGFβ1 in the absence or presence of 100 ng/mL recombinant human myeloid-derived growth factor (MYDGF). SMAD phosphorylation (activation) was determined by immunoblotting. The small molecule ALK5/TGFβ type I receptor inhibitor SB431542 (10 μmol/L) was used as positive control. Results are shown in FIG. 3.

### Example 21

Mouse myeloid-derived growth factor inhibits migration of mouse embryonic fibroblasts (MEFs). MEFs were grown to confluency. Monolayers were scratched with a 200 μL pipet tip, washed, and cultured for 16 hours in the absence or presence of 2 ng/mL mouse transforming growth factor β1 (Tgfß1) and/or 100 ng/mL recombinant mouse myeloid-derived growth factor (Mydgf). Before (0 hours) and after stimulation (16 hours), digital phase contrast images were captured. Recovery (%) was calculated as [(cell free area at 0 hours−cell free area at 16 hours)/cell free area at 0 hours]×100. Results are shown in table 21 below.

TABLE 21
Migration after scratch injury of mouse embryonic fibroblasts cultured
in the absence (control) or presence of Tgfβ1 and/or Mydgf

| | Number of | | |
|---|---|---|---|
| | independent | Recovery | |
| | experiments | Mean ± SEM (%) | P value |
| Control | 5 | 24.7 ± 0.3 | |
| Mydgf | 5 | 22.3 ± 0.6 | <0.01 vs. control |
| TGFβ1 | 5 | 31.3 ± 1.1 | |
| TGFβ1 + Mydgf | 5 | 25.0 ± 0.9 | <0.01 vs. Tgfβ1 |

### Example 22

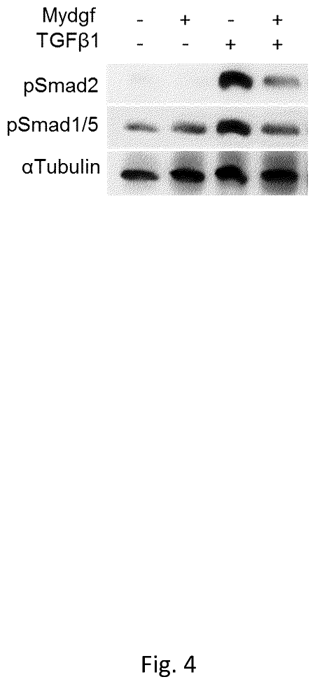

Mouse myeloid-derived growth factor inhibits transforming growth factor β1 (Tgfß1)-stimulated Smad phosphorylation in mouse embryonic fibroblasts (MEFs). MEFs were stimulated for 30 min with 2 ng/mL recombinant mouse Tgfß1 in the absence or presence of 100 ng/mL recombinant mouse myeloid-derived growth factor (Mydgf). Smad phosphorylation (activation) was determined by immunoblotting. Results are shown in FIG. 4.

### Example 23

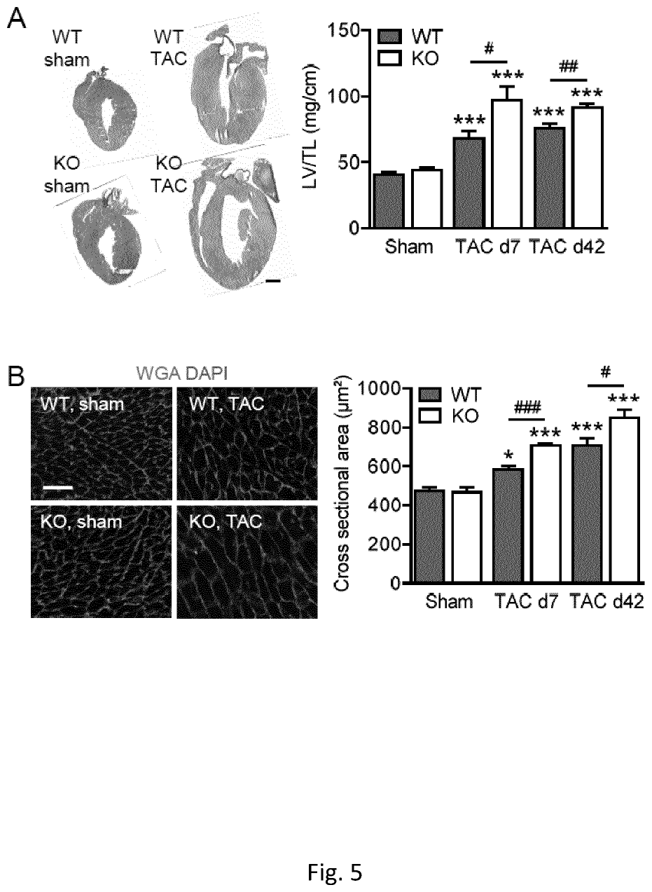

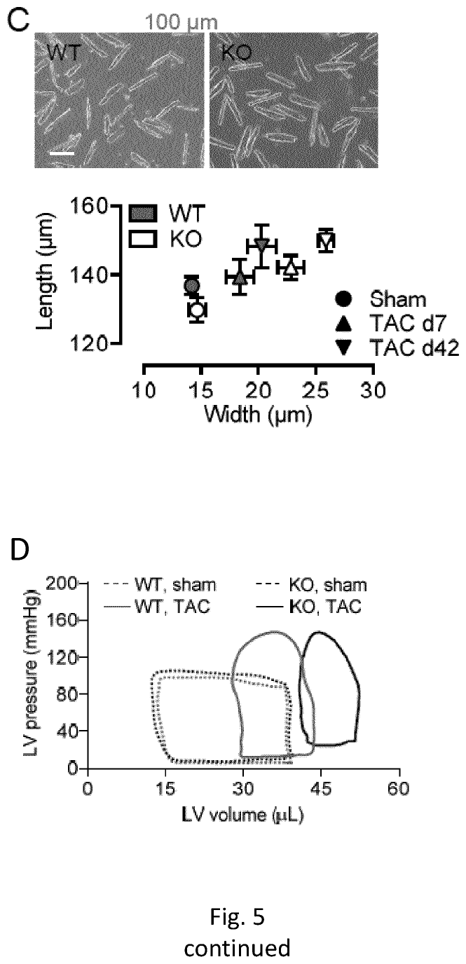

Inflammatory cell-derived MYDGF attenuates cardiac remodeling during pressure overload. To explore the function of MYDGF during pressure overload, Mydgf KO mice and their WT littermates were subjected to TAC surgery. Mydgf KO mice breed and develop normally and display no overt cardiovascular phenotype at baseline (Korf-Klingebiel 2015). Pressure gradients across the site of aortic constriction and right-to-left common carotid artery peak blood flow velocity ratios were similar in WT and KO mice. Despite comparable aortic stenosis severity, KO mice developed more pronounced LV hypertrophy than WT mice (FIG. 5A), with greater increases in cardiomyocyte size revealed by histological and single-cell examination 7 and 42 days after TAC (FIGS. 5B and 5C). Compared to WT mice, KO mice showed a stronger decline in Myh6 (alpha myosin heavy chain) mRNA expression at day 7, greater increases in Myh7 (beta myosin heavy chain) mRNA at days 7 and 42, more elevated Nppa (natriuretic peptide type A) mRNA at day 42, and a similar rise in Nppb (natriuretic peptide type B) mRNA at both time points.

LV pressure-volume measurements showed that KO mice develop more pronounced systolic and diastolic dysfunction after TAC than do WT controls (FIG. 5D; table 22).

TABLE 22
Left ventricular volume measurements in Mydgf wild-type and knockout mice

| | WT, sham | KO, sham | WT, TAC | KO, TAC |
|---|---|---|---|---|
| Heart rate (min⁻¹) | 499 ± 15 | 500 ± 19 | 483 ± 7 | 486 ± 10 |
| LVEDP (mmHg) | 5 ± 1 | 5 ± 1 | 9 ± 1 | 19 ± 4**^{,#} |
| LVESP (mmHg) | 98 ± 4 | 103 ± 4 | 150 ± 7*** | 149 ± 11** |
| LVEDV (μL) | 40 ± 3 | 41 ± 3 | 45 ± 3 | 50 ± 7 |
| LVESV (μL) | 14 ± 1 | 14 ± 2 | 28 ± 3 | 42 ± 6*** |
| LVEF (%) | 71 ± 2 | 71 ± 3 | 48 ± 3*** | 29 ± 5***^{,##} |
| dP/dtₘₐₓ (mmHg/s) | 9,836 ± 457 | 10,472 ± 472 | 7,536 ± 323** | 6,455 ± 273*** |
| dP/dtₘᵢₙ (mmHg/s) | −8,645 ± 382 | −8,764 ± 341 | −7,923 ± 358 | −5,341 ± 406***^{,###} |
| τ (ms) | 7.2 ± 0.3 | 7.2 ± 0.4 | 8.1 ± 0.4 | 12.0 ± 1.1***^{,##} |

Transverse aortic constriction (TAC) was induced in Mydgf wild-type (WT) and knockout (KO) mice (8 mice in both groups). Additional mice underwent sham surgery (6 mice per group). Left ventricular (LV) pressure-volume loops were recorded after 7 d. LVEDP, LV end-diastolic pressure; LVESP, LV end-systolic pressure; LVEDV, LV end-diastolic volume; LVESV, LV end-systolic volume; LVEF, LV ejection fraction; dP/dtₘₐₓ, maximum rate of pressure change in the left ventricle; dP/dtₘᵢₙ, minimum rate of pressure change in the left ventricle.
**P < 0.01,
***P < 0.001 vs same genotype sham;
^{#}P < 0.05,
^{##}P < 0.01,
^{###}P < 0.001 KO TAC vs WT TAC (2-way ANOVA with Tukey’s post hoc test).

Additionally, bone marrow-chimeric mice were generated to specifically address the importance of inflammatory cell-derived MYDGF in modulating LV hypertrophy and heart function during pressure overload. Transplanting WT bone marrow cells (BMCs) into KO mice inhibited LV hypertrophy (FIG. 6A) and cardiomyocyte hypertrophy (FIG. 6B), enhanced myocardial capillarization (FIG. 6C), and attenuated LV dilatation (FIG. 6D) and systolic dysfunction after TAC challenge (FIG. 6E). Conversely, transplanting KO BMCs into WT mice augmented hypertrophy, reduced capillary density, and worsened LV remodeling and systolic dysfunction (FIG. 6A through 6E). Lentiviral gene transfer was employed to enable inducible inflammatory cell-specific overexpression of MYDGF in WT mice (FIG. 6F).

In mice transplanted with Lenti.MYDGF-transduced BMCs, adding doxycycline to the drinking water enhanced MYDGF protein expression in BMCs (5.8±2.2 fold vs Lenti.MYDGF without doxycycline) and splenocytes (9.1±2.7 fold vs Lenti.MYDGF without doxycycline) (FIG. 6G). After TAC surgery, doxycycline-treated Lenti.MYDGF mice had higher MYDGF plasma concentrations (FIG. 6H) and greater LV MYDGF expression levels (FIG. 6I) than doxycycline-treated Lenti.control mice. Doxycycline-treated Lenti.MYDGF mice developed milder LV hypertrophy after TAC surgery (FIG. 6J), with smaller cardiomyocytes (FIG. 6K), somewhat higher capillary density (P=0.11, FIG. 6L), less LV dilatation (FIG. 6M), and more preserved systolic function (FIG. 6N) than doxycycline-treated Lenti.control mice. It is therefore concluded that inflammatory cell-derived MYDGF is required and sufficient for limiting maladaptive hypertrophy and remodeling during chronic pressure overload.

### Example 24

Phosphoproteome Analysis Identifies PIM1 as a Signaling Target of MYDGF in Cardiomyocytes. An in vitro hypertrophy model was established to assess whether MYDGF targets cardiomyocytes directly. ET1 stimulation for 24 hours increased neonatal rat ventricular cardiomyocyte (NRCM) size (FIG. 7B), protein content (FIG. 7C), and Myh7 and Nppa mRNA gene expression (FIG. 7D). While recombinant MYDGF alone did not affect these endpoints, cotreatment with MYDGF almost completely prevented the hypertrophic response to ET1; MYDGF's antihypertrophic effects were concentration-dependent and saturable with a half-maximal inhibitory concentration of 7.6 ng/mL (FIG. 7B). MYDGF similarly inhibited Ang II-induced hypertrophy but did not attenuate hypertrophy in response to IGF1 (FIG. 7A). In contrast to ET1 and Ang II promoting a pathological type of cardiac hypertrophy, Insulin-like growth factor 1 (IGF1) promotes a physiological type of cardiac hypertrophy in neonatal rat cardiomyocytes, which is not attenuated by MYDGF.

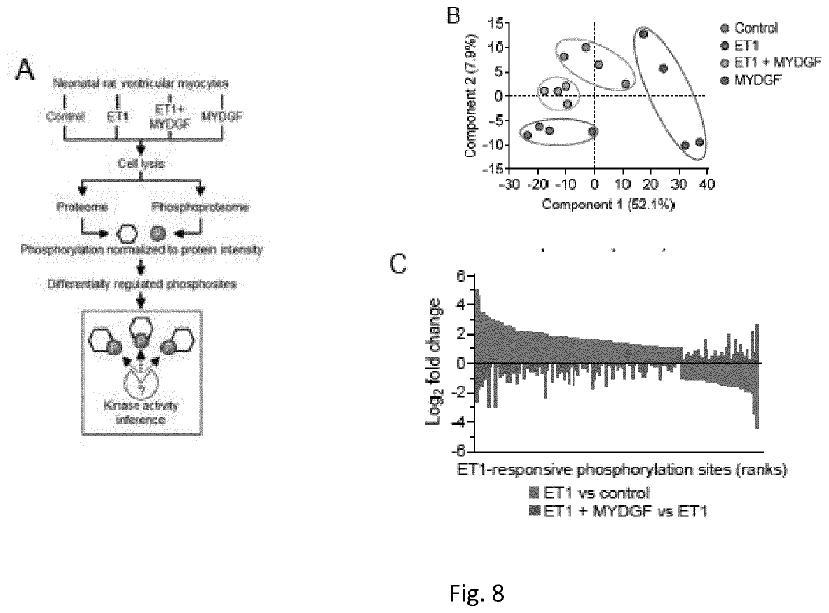

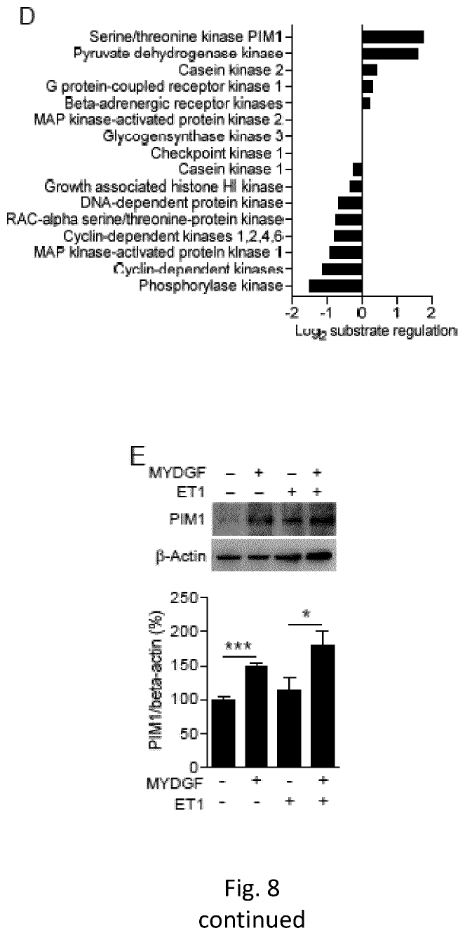

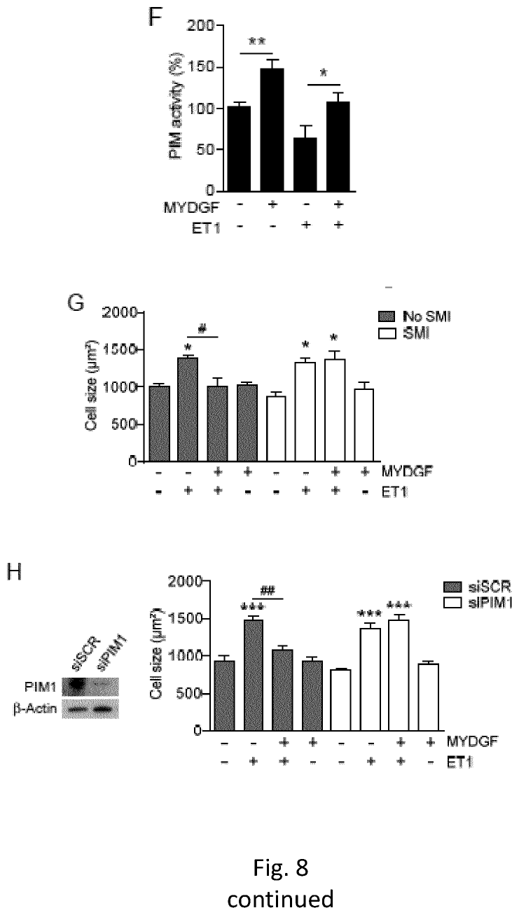

To identify signaling intermediate(s) activated by MYDGF, a phosphoproteome analysis followed by computational substrate-based kinase activity inference was performed (FIG. 8A) (Hogrebe et al. Nat Commun. 2018; 9:1045; Strasser et al. Integr Biol. 2019; 11:301-314). High-content LC-MS/MS data were collected 8 hours after stimulating NRCMs with ET1 in the absence or presence of MYDGF. Of 2,308 quantified phosphorylation sites distributed among 1,110 different proteins, 423 were differentially phosphorylated across the four experimental conditions when scaled to corresponding differences in protein abundance.

Unsupervised hierarchical clustering and principal component analysis (FIG. 8B) indicated that biological replicates from each condition clustered together, thus validating the workflow's reproducibility. The four conditions were well segregated in principle component space, indicating that they were associated with distinct phosphoproteome signatures. Using Euclidean distance as a similarity metric, we observed that NRCMs cotreated with ET1 and MYDGF displayed an intermediate phenotype positioned between ET1 and unstimulated control (FIG. 8B). Indeed, MYDGF reversed many of the phosphoproteomic changes induced by ET1: phosphorylation sites that were more intensely phosphorylated in ET1-treated cells (compared with unstimulated control) tended to be less intensely phosphorylated in cells costimulated with ET1 and MYDGF (compared with ET1 alone), and vice versa (FIG. 8C). Applying prior knowledge of kinase-substrate interactions (Hornbeck et al. Nucleic Acids Res. 2015; 43:D512-520) to the phosphoproteome datasets, kinase activity changes induced by MYDGF in ET1-treated cells were inferred. Based on differentially regulated phosphorylation sites and enrichment of their respective kinase substrate motifs, MYDGF was predicted to alter the activities of several protein kinases, including a strong activation of the serine/threonine kinase PIM1 (FIG. 8D).

PIM1 and its isoforms, PIM2 and PIM3, have similar substrate preferences but different tissue expression profiles (Selten et al. Cell. 1986; 46:603-611; Qian et al. J Biol Chem. 2005; 280:6130-6137; Nawijn et al. Nat Rev Cancer. 2011; 11:23-34), PIM1 being the predominant isoform in the heart (Muraski et al. Nat Med. 2007; 13:1467-1475). PIM kinases are constitutively active and are regulated at the level of protein expression (Nawijn 2011). In an earlier study, overexpressing PIM1 protected NRCMs from ET1-induced hypertrophy (Muraski 2007), and PIM1 therefore emerged as a potential candidate mediating MYDGF effects in cardiomyocytes. Substantiating our phosphoproteome data, MYDGF increased PIM1 expression (FIG. 8E) and kinase activity (FIG. 8F) in unstimulated and ET1-stimulated NRCMs. MYDGF's antihypertrophic effects were curtailed by the small-molecule PIM kinase inhibitor SMI4a (Beharry et al. Mol Cancer Ther. 2009; 8:1473-1483; Xia et al. J Med Chem. 2009; 52:74-86 (FIG. 8G) and by siRNA-mediated PIM1 downregulation (FIG. 8H), showing that MYDGF, indeed, signals via PIM1.

### Example 25

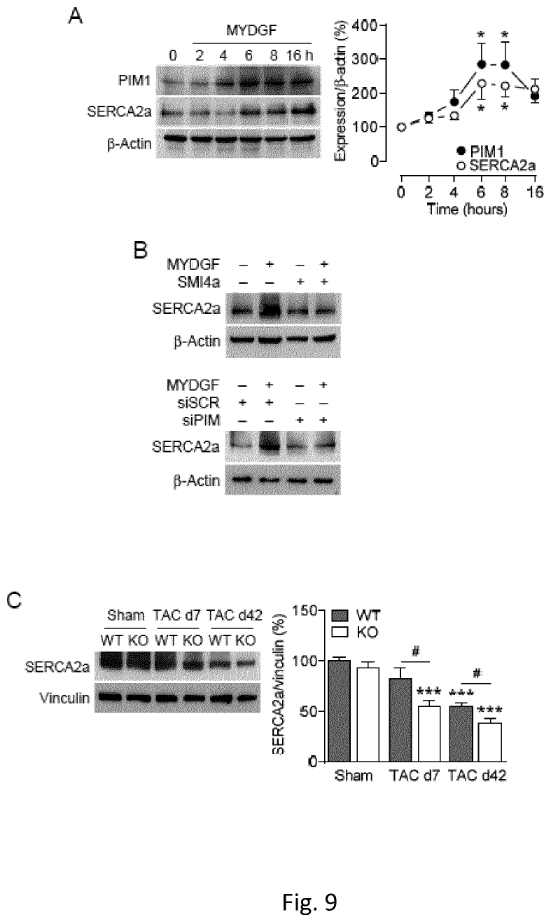

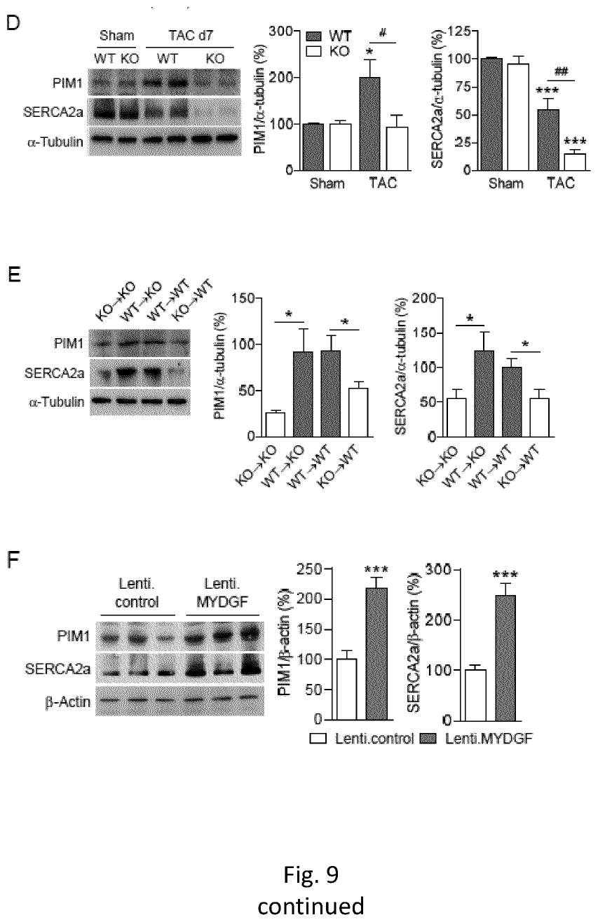

MYDGF enhances SERCA2a expression in cardiomyocytes via PIM1. Abnormalities in Ca²⁺ cycling, resulting in slower cardiomyocyte contraction and relaxation, are common in the hypertrophied and failing heart (Houser et al. J Mol Cell Cardiol. 2000; 32:1595-1607). Reduced sarco/endoplasmic reticulum Ca²⁺-ATPase 2a (SERCA2a) expression and/or activity, leading to impaired Ca²⁺ sequestration into the sarco/endoplasmic reticulum, may contribute to defective Ca²⁺ regulation in heart failure (Luo et al. Circ Res. 2013; 113:690-708). As PIM1 has previously been reported to stimulate SERCA2a expression (Muraski et al. Nat Med. 2007; 13:1467-1475), it was explored whether SERCA2a abundance is controlled by MYDGF. Indeed, increases in PIM1 expression in MYDGF-stimulated NRCMs were paralleled by increases in Serca2a protein expression (FIG. 9A). Inhibition or siRNA-mediated downregulation of PIM1 prevented the rise in Serca2a, showing that MYDGF controls SERCA2a expression via PIM1 (FIG. 9B).

In vivo, LV myocardial SERCA2a protein levels were comparable in sham-operated WT and Mydgf KO mice (FIG. 9C). After TAC surgery, SERCA2a expression in WT mice was still preserved on day 7 (−17%, P=0.14) but diminished on day 42. At both time points, SERCA2a expression was significantly lower in KO than in WT mice (FIG. 9C). Sham-operated WT and KO mice had similar PIM1 and SERCA2a protein expression levels in isolated LV cardiomyocytes (FIG. 9D). PIM1 expression in cardiomyocytes increased after TAC in WT but not in KO mice. Inability of KO mice to upregulate PIM1 after TAC was associated with strongly reduced SERCA2a abundance in cardiomyocytes (FIG. 9D).

To specifically define whether inflammatory cell-derived MYDGF regulates PIM1 and SERCA2a expression in the pressure-overloaded heart, Mydgf bone marrow-chimeric and bone marrow-conditional transgenic mice were subjected to TAC surgery. Transplanting WT BMCs into Mydgf KO mice enhanced, whereas transplanting KO BMCs into WT mice diminished, LV PIM1 and SERCA2a abundance after TAC (FIG. 9E). Also, doxycycline-treated Lenti.MYDGF mice had higher LV PIM1 and SERCA2a expression levels than doxycycline-treated Lenti.control mice (FIG. 9F). It is concluded that inflammatory cell-derived MYDGF is required and sufficient for enhancing PIM1 and SERCA2a expression in the pressure-overloaded left ventricle.

### Example 26

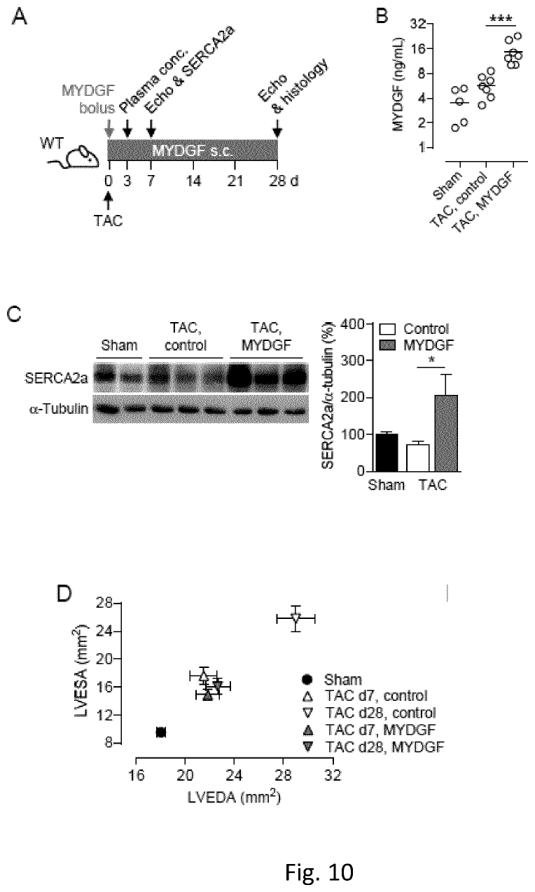

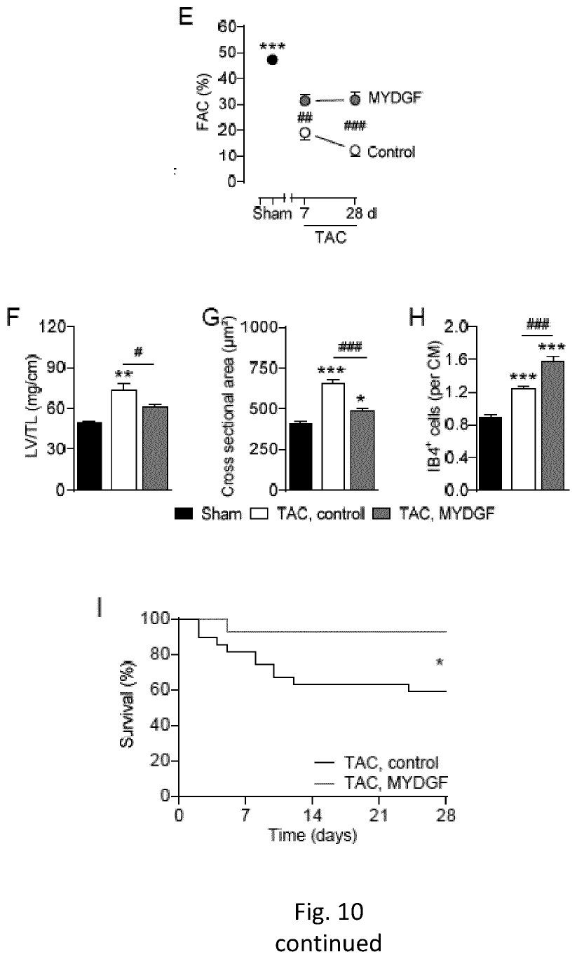

The therapeutic potential of MYDGF during pressure overload was explored. Mice were treated with recombinant MYDGF for 28 days after TAC surgery using subcutaneously implanted osmotic minipumps to ensure continued protein delivery (10 μg/day) (FIG. 10A). Three days after TAC, MYDGF-treated mice had higher MYDGF plasma concentrations than control mice treated with diluent-only (FIG. 10B). Seven days after TAC, MYDGF-treated mice had more abundant SERCA2a protein expression in isolated ventricular cardiomyocytes (FIG. 10C) and, over the course of 28 days, these animals developed less pronounced LV dilatation (FIG. 10D) and systolic dysfunction (FIG. 10E). The antiremodeling effects were associated with an attenuated hypertrophic response (FIG. 10F) with smaller cardiomyocytes (FIG. 10G), increased capillary density in the LV myocardium (FIG. 10H), and a marked survival benefit (FIG. 10I).

In particular examples 23, 24, 25 and 26 show that MYDGF protects against pressure overload-induced heart failure in mice. Implementing acute pressure overload by TAC surgery triggered a swift increase in MYDGF abundance in the left ventricle with monocytes and macrophages emerging as the main MYDGF-producing cell types. Recruitment and differentiation of circulating CCR2^{high} monocytes and proliferation of cardiac-resident macrophages lead to a notable expansion of the macrophage pool during pressure overload.

After TAC challenge, Mydgf KO mice developed more severe LV hypertrophy with larger cardiomyocytes than wild-type mice. Greater hypertrophy in KO mice was characterized by impaired Ca²⁺ cycling and sarcomere function, more marked fetal gene activation, reduced microvascular density, enhanced interstitial fibrosis, and intensified LV dilatation and systolic and diastolic dysfunction, all hallmarks of a maladaptive response to pressure overload.

Exercise training did not provoke myeloid cell expansion in the heart. Accordingly, MYDGF expression was comparably low in left ventricles from trained and untrained mice, and trained Mydgf KO mice developed physiological hypertrophy like their wild-type littermates.

Acting on cardiomyocytes, MYDGF diminished cellular hypertrophy and improved Ca²⁺ cycling and sarcomere function by enhancing SERCA2a expression. Phosphoproteomics pinpointed the constitutively active serine/threonine kinase PIM1 as one potential MYDGF downstream target. Indeed, inhibiting or downregulating PIM1 abolished MYDGF's antihypertrophic and SERCA2a-inducing effects in cultured cardiomyocytes.

It has been shown that recombinant MYDGF treatment mediates beneficial effects on LV shape, systolic function, and survival during persistent afterload stress.

1. Myeloid-derived growth factor (MYDGF) or a fragment or a variant thereof exhibiting the biological function of MYDGF, for use in treating or preventing fibrosis or hypertrophy.

2. MYDGF or a fragment or a variant thereof exhibiting the biological function of MYDGF, for use in treating or preventing heart failure, preferably wherein the heart failure is chronic heart failure.

3. The MYDGF or a fragment or a variant thereof for use according to claim 2, wherein the heart failure or chronic heart failure is heart failure with preserved ejection fraction (HFpEF), heart failure with reduced ejection fraction (HFrEF), heart failure with mid-range ejection fraction (HFmrEF).

4. The MYDGF for use according to claim 1, 2 or 3, wherein the MYDGF comprises:
(i) SEQ ID NO: 1; or
(ii) a fragment or variant of SEQ ID NO: 1 exhibiting the biological function of MYDGF, and
wherein the variant comprises an amino acid sequence with at least 85% amino acid sequence identity to SEQ ID NO:1.

5. The growth factor MYDGF for use according to any one of claims 1 or 4, wherein the fibrosis is fibrosis of the heart and/or lung, preferably wherein the fibrosis is an interstitial lung disease, preferably progressive fibrosing interstitial lung disease, and more preferably idiopathic pulmonary fibrosis.

6. The growth factor MYDGF for use according to claim 1 or 4, wherein the hypertrophy is hypertrophy of cardiomyocytes.

7. A nucleic acid encoding growth factor MYDGF or a fragment or a variant thereof exhibiting the biological function of MYDGF, for use in treating or preventing fibrosis or hypertrophy.

8. A nucleic acid encoding growth factor MYDGF or a fragment or a variant thereof exhibiting the biological function of MYDGF, for use in treating or preventing heart failure.

9. The nucleic acid for use according to claim 7 or 8, wherein the nucleic acid encodes an amino acid sequence having at least 85% sequence identity to SEQ ID NO:1.

10. A vector comprising the nucleic acid of claim 7 or 8, for use in treating or preventing fibrosis or hypertrophy.

11. A vector comprising the nucleic acid of claim 8 or 9, for use in treating or preventing heart failure.

12. A host cell comprising the nucleic acid of claim 7 or the vector according to claim 10 and expressing the nucleic acid, for use in treating or preventing fibrosis or hypertrophy.

13. A host cell comprising the nucleic acid of claim 8 or the vector according to claim 11 and expressing the nucleic acid, for use in treating or preventing heart failure.

14. A pharmaceutical composition comprising MYDGF of any of claims 1, 4 to 6, the nucleic acid of claim 6 or 8, the vector of claim 9, or the host cell of claim 11, and optionally a suitable pharmaceutical excipient, for use in treating or preventing fibrosis or hypertrophy.

15. A pharmaceutical composition comprising MYDGF of any of claims 2 to 6, the nucleic acid of claim 8 or 9, the vector of claim 11, or the host cell of claim 13, and optionally a suitable pharmaceutical excipient, for use in treating or preventing heart failure.

16. The pharmaceutical composition for use of claim 14 or 15, wherein said pharmaceutical composition is administered through the oral, intravenous, subcutaneous, intramucosal, intraarterial, intramuscular or intracoronary route.

17. The pharmaceutical composition for use of claim 16, wherein the administration is through one or more bolus injection(s) and/or infusion(s).

18. A method of treating fibrosis, comprising administering to a patient in need thereof a therapeutically effective amount of MYDGF or fragment or variant thereof exhibiting the biological function of MYDGF.

19. The method according to claim 18, wherein the MYDGF comprises:
(i) SEQ ID NO: 1; or
(ii) a fragment or variant thereof exhibiting the biological function of MYDGF of SEQ ID NO: 1, and
wherein the variant comprises an amino acid sequence with at least 85% amino acid sequence identity to SEQ ID NO:1.

20. The method according to claim 18, wherein the fibrosis is fibrosis of the heart, and/or lung, preferably wherein the fibrosis is an interstitial lung disease, preferably progressive fibrosing interstitial lung disease, and more preferably idiopathic pulmonary fibrosis.

21. The method according to claim 18, wherein the MYDGF or fragment or variant thereof exhibiting the biological function of MYDGF is administered through one or more bolus injection(s) and/or infusion(s), preferably in a pharmaceutically accepted carrier.

22. A method of treating hypertrophy, comprising administering to a patient in need thereof a therapeutically effective amount of MYDGF or fragment or variant thereof exhibiting the biological function of MYDGF.

23. The method according to claim 22, wherein the MYDGF comprises:
(i) SEQ ID NO: 1; or
(ii) a fragment or variant thereof exhibiting the biological function of MYDGF of SEQ ID NO: 1, and
wherein the fragment or variant comprises an amino acid sequence with at least 85% amino acid sequence identity to SEQ ID NO:1.

24. The method according to claim 22, wherein the hypertrophy is hypertrophy of cardiomyocytes.

25. The method according to claim 22, wherein the MYDGF or fragment or variant thereof exhibiting the biological function of MYDGF is administered through one or more bolus injection(s) and/or infusion(s), preferably in a pharmaceutically accepted carrier.

26. A method of treating or preventing heart failure, comprising administering to a patient in need thereof a therapeutically effective amount of growth factor MYDGF or fragment or variant thereof exhibiting the biological function of MYDGF, preferably wherein the heart failure is chronic heart failure.

27. The method of treating heart failure according to claim 26, wherein the heart failure or chronic heart failure is HFpEF or HFrEF, preferably wherein the HFpEF is Stage C or Stage D HFpEF, or wherein the HFrEF is Stage C or Stage D HFrEF.

28. The method according to claim 26 or 27, wherein the MYDGF comprises:
(i) SEQ ID NO: 1; or
(ii) a fragment or variant thereof exhibiting the biological function of MYDGF of SEQ ID NO: 1, and
wherein the fragment or variant comprises an amino acid sequence with at least 85% amino acid sequence identity to SEQ ID NO:1.
